SEQUENCE LISTING (7 sequences)

SEQ ID NO: 1 - protein, 601 aa, Severe acute respiratory syndrome coronavirus 2
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 2 - protein, 597 aa, Human coronavirus 229E
AAGLCVVCGSQTVLRCGDCLRRPMLCTKCAYDHVFGTDHKFILAITPYVCNTSGCNVNDVTKLYLGGLNYYCVDHKPHLSFPLCSAGNVFGLYKSSALGSMDIDVFNKLSTSDWSDIRDYKLANDAKESLRLFAAETVKAKEESVKSSYAYATLKEIVGPKELLLLWESGKAKPPLNRNSVFTCFQITKDSKFQVGEFVFEKVDYGSDTVTYKSTATTKLVPGMLFILTSHNVAPLRAPTMANQEKYSTIYKLHPSFNVSDAYANLVPYYQLIGKQRITTIQGPPGSGKSHCSIGIGVYYPGARIVFTACSHAAVDSLCAKAVTAYSVDKCTRIIPARARVECYSGFKPNNNSAQYVFSTVNALPEVNADIVVVDEVSMCTNYDLSVINQRISYKHIVYVGDPQQLPAPRVLISKGVMEPIDYNVVTQRMCAIGPDVFLHKCYRCPAEIVNTVSELVYENKFVPVKEASKQCFKIFERGSVQVDNGSSINRRQLDVVKRFIHKNSTWSKAVFISPYNSQNYVAARLLGLQTQTVDSAQGSEYDYVIFAQTSDTAHACNANRFNVAITRAKKGIFCIMSDRTLFDALKFFEITMTDLQ

SEQ ID NO: 3 - protein, 602 aa, Human coronavirus NL63
AAGLCVVCGSQTVLRCGDCLRKPMLCTKCAYDHVFGTDHKFILAITPYVCNASGCGVSDVKKLYLGGLNYYCTNHKPQLSFPLCSAGNIFGLYKNSATGSLDVEVFNRLATSDWTDVRDYKLANDVKDTLRLFAAETIKAKEESVKSSYAFATLKEVVGPKELLLSWESGKVKPPLNRNSVFTCFQISKDSKFQIGEFIFEKVEYGSDTVTYKSTVTTKLVPGMIFVLTSHNVQPLRAPTIANQEKYSSIYKLHPAFNVSDAYANLVPYYQLIGKQKITTIQGPPGSGKSHCSIGLGLYYPGARIVFVACAHAAVDSLCAKAMTVYSIDKCTRIIPARARVECYSGFKPNNTSAQYIFSTVNALPECNADIVVVDEVSMCTNYDLSVINQRLSYKHIVYVGDPQQLPAPRVMITKGVMEPVDYNVVTQRMCAIGPDVFLHKCYRCPAEIVNTVSELVYENKFVPVKPASKQCFKVFFKGNVQVDNGSSINRKQLEIVKLFLVKNPSWSKAVFISPYNSQNYVASRFLGLQIQTVDSSQGSEYDYVIYAQTSDTAHACNVNRFNVAITRAKKGIFCVMCDKTLFDSLKFFEIKHADLHSSQVC

SEQ ID NO: 4 - protein, 600 aa, Human coronavirus OC43
SVGACVVCSSQTSLRCGSCIRKPLLCCKCCYDHVMATDHKYVLSVSPYVCNAPGCDVNDVTKLYLGGMSYYCEDHKPQYSFKLVMNGLVFGLYKQSCTGSPYIDDFNRIASCKWTDVDDYILANECTERLKLFAAETQKATEEAFKQSYASATIQEIVSERELILSWEIGKVKPPLNKNYVFTGYHFTKNGKTVLGEYVFDKSELTNGVYYRATTTYKLSVGDVFVLTSHSVANLSAPTLVPQENYSSIRFASVYSVLETFQNNVVNYQHIGMKRYCTVQGPPGTGKSHLAIGLAVFYCTARVVYTAASHAAVDALCEKAYKFLNINDCTRIVPAKVRVECYDKFKINDTTRKYVFTTINALPEMVTDIVVVDEVSMLTNYELSVINARIRAKHYVYIGDPAQLPAPRVLLSKGTLEPKYFNTVTKLMCCLGPDIFLGTCYRCPKEIVDTVSALVYENKLKAKNESSSLCFKVYYKGVTTHESSSAVNMQQIYLINKFLKANPLWHKAVFISPYNSQNFAAKRVLGLQTQTVDSAQGSEYDYVIYSQTAETAHSVNVNRFNVAITRAKKGILCVMSNMQLFEALQFTTLTLDKVPQAVET

SEQ ID NO: 5 - protein, 600 aa, Human coronavirus HKU1
SVGACVVCSSQTSLRCGSCIRKPLLCCKCCYDHVMATNHKYVLSVSPYVCNAPNCDVSDVTKLYLGGMSYYCENHKPHYSFKLVMNGMVFGLYKQSCTGSPYIDDFNKIASCKWTEVDDYVLANECIERLKLFAAETQKATEEAFKQSYASATIQEIVSDREVILCWETGKVKPPLNKNYVFTGYHFTSTGKTVLGEYVFDKSELTNGVYYRATTTYKLSIGDVFVLTSHSVASLSAPTLVPQENYASIRFSSVYSVPLVFQNNVANYQHIGMKRYCTVQGPPGTGKSHLAIGLAVYYYTARVVYTAASHAAVDALCEKAYKFLNINDCTRIIPAKVRVDCYDKFKINDTTCKYVFTTINALPELVTDIVVVDEVSMLTNYELSVINARIKAKHYVYIGDPAQLPAPRVLLSKGSLEPRHFNSITKIMCCLGPDIFLGNCYRCPKEIVETVSALVYDNKLKAKNDNSSLCFKVYFKGQTTHESSSAVNIQQIYLISKFLKANPVWNSAVFISPYNSQNYVAKRVLGVQTQTVDSAQGSEYDYVIYSQTAETAHSVNVNRFNVAITRAKKGIFCVMSNMQLFESLNFITLPLDKIQNQTLP

SEQ ID NO: 6 - protein, 598 aa, Middle East respiratory syndrome-related coronavirus
AVGSCVVCHSQTSLRCGTCIRRPFLCCKCCYDHVIATPHKMVLSVSPYVCNAPGCGVSDVTKLYLGGMSYFCVDHRPVCSFPLCANGLVFGLYKNMCTGSPSIVEFNRLATCDWTESGDYTLANTTTEPLKLFAAETLRATEEASKQSYAIATIKEIVGERQLLLVWEAGKSKPPLNRNYVFTGYHITKNSKVQLGEYIFERIDYSDAVSYKSSTTYKLTVGDIFVLTSHSVATLTAPTIVNQERYVKITGLYPTITVPEEFASHVANFQKSGYSKYVTVQGPPGTGKSHFAIGLAIYYPTARVVYTACSHAAVDALCEKAFKYLNIAKCSRIIPAKARVECYDRFKVNETNSQYLFSTINALPETSADILVVDEVSMCTNYDLSIINARIKAKHIVYVGDPAQLPAPRTLLTRGTLEPENFNSVTRLMCNLGPDIFLSMCYRCPKEIVSTVSALVYNNKLLAKKELSGQCFKILYKGNVTHDASSAINRPQLTFVKNFITANPAWSKAVFISPYNSQNAVSRSMLGLTTQTVDSSQGSEYQYVIFCQTADTAHANNINRFNVAITRAQKGILCVMTSQALFESLEFTELSFTNYKLQ

SEQ ID NO: 7 - protein, 601 aa, Severe acute respiratory syndrome coronavirus 1
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKIGILCIMSDRDLYDKLQFTSLEIPRRNVATLQ

(19) United States
(12) Patent Application Publication
(10) Pub. No.: US 2026/0151396 A1
(43) Pub. Date: Jun. 4, 2026

(54) Inhibitors of Viral Helicases Binding to a Novel Allosteric Binding Site

(71) Applicant: Eisbach Bio GmbH, Martinsried (DE)

(72) Inventors: William M. Menzer, München (DE); Gunnar Knobloch, Planegg (DE); Adrian Schomburg, Gräfelfing (DE); Corinna Lieleg, Garching (DE); Peter Sennhenn, München (DE)

(21) Appl. No.: 18/703,992

(22) PCT Filed: Oct. 28, 2022
(86) PCT No.: PCT/EP2022/080307

§ 371 Date: Apr. 23, 2024

(30) Foreign Application Priority Data
Oct. 29, 2021 (EP) 21205666.7

(51) Int. Cl. A61K 31/517; A61K 31/4178; A61K 31/454; A61P 31/14; C07D 239/84; C07D 239/94; C07D 401/04; C07D 403/04; C07D 403/06; C07D 403/12; C07D 417/12; C07D 417/14

(52) U.S. Cl. CPC A61K 31/517; A61K 31/4178; A61K 31/454; A61P 31/14; C07D 239/84; C07D 239/94; C07D 401/04; C07D 403/04; C07D 403/06; C07D 403/12; C07D 417/12; C07D 417/14

(57) ABSTRACT

This application provides small molecular compounds that inhibit viral helicases in particular the Nsp13 helicase of coronavirus and the use of such compounds for preventing and treating viral infection, in particular for treating viral infection with a coronavirus.

This application provides small molecular compounds that inhibit viral helicases in particular the Nsp13 helicase of coronavirus and the use of such compounds for preventing and treating viral infection, in particular for treating viral infection with a coronavirus.

### BACKGROUND OF THE INVENTION

The viral pandemic caused by the severe respiratory syndrome coronavirus 2 (SARS-CoV-2) is one of the worst outbreaks of respiratory diseases in the last century, resulting in more than 4.500.000 deaths within a year since the first appearance of the viral infection in the Chinese province of Wuhan in December 2019. The pandemic has had global socio-economic ramifications, affecting the lives of billions of people everywhere. Yet, to date there is no approved efficacious therapy for the treatment of the disease caused by SARS-CoV-2 infections, COVID-19. The most promising therapeutic opportunities thus far are repurposing approaches of known antiviral drugs targeting proteases and polymerases that are essential for the replication of viruses, such as remdesivir (HIV), indinavir (HIV), saquinavir (HIV) and lopinavir/ritonavir (HIV, hepatitis C). However, to date there is no targeted approved treatment for COVID-19, and current therapies are focused only on the alleviation of symptoms which may include fever, dry cough, and pneumonia. Thus, there is a great medical need for targeted treatments of human coronavirus infections, especially in the light of current reports showing declining antibody titers in patients post COVID-19 infections, questioning the efficacy of vaccines (Long, Q, et al. (2020) Nat. Med., https://doi.org/10.1038/s41591-020-0965-6).

Coronaviruses are single-stranded, positive-strand RNA viruses, of which seven types have been shown to infect humans: 229E, NL63, OC43, HKU1, MERS-CoV, SARS-CoV and SARS-CoV-2. The symptoms of infections range from mild respiratory distress to more severe illnesses that can be lethal. The 29.9 kb SARS-CoV-2 genome contains at least six open reading frames (ORFs), of which the first ORF (ORF1a/b) constitutes >70% of the genome. It encodes for 16 non-structural proteins (nsp1-16) which have been shown to play a major role in viral replication. The four main structural proteins including spike, envelope, membrane and nucleocapsid are encoded by ORFs near the 3′-end of the genome. These proteins are important for virion assembly and cell entry, ultimately causing cellular coronavirus infection. Additionally, specific structural and accessory proteins such as HE protein are also encoded by the coronavirus genome (Chen, Y. et al. (2020) J. Med. Virol., 92:418-423). Interestingly, SARS-CoV-2 shares more than ˜80% identity on the nucleotide level to the first identified less infectious but more lethal SARS epidemic virus (L. E. Gralinski, V. D. Menachery (2020) Viruses, 12: 135).

Surprisingly, the structural proteins of coronaviruses show great variability among the different coronavirus species while the key non-structural proteins (NSPs), and particularly the helicase Nsp13, have been shown to be much more conserved. In particular the viral helicases of the SARS-CoV and SARS-CoV-2 Coronaviridae show more than 99% sequence identity (see FIG. 1). Quite strikingly there is a close evolutionary relationship among viral helicases and in particular the viral helicases of the Coronaviridae (see FIG. 2). The amino acid sequence comprising the allosteric pocket is also highly conserved among the class of Pisoniviricetes, in particular the order Nidovirales, more particularly the suborder of Cornidovirineae and most particular the family of Coronaviridae and shows at least 57% sequence identity and more than 28% similarity among Coronaviridae (see FIG. 3). The structure of the MERS-CoV and the SARS-CoV and SARS-CoV2 Nsp13 helicases have been solved by X-ray crystallography (see Hao, W. et al. (2017) PLos Pathog, 13: e1006474-e1006474 and Jia, Z. et al. (2019) Nucleic Acids Res 47: 6538-6550) FIG. 4), showing a high degree in structural similarity with overall RMSD values in the range of 1.15 and 1.6 Å². The proteins belong to the SF1 superfamily of helicases and consist of five domains: an N-terminal zinc binding domain (ZBD), a helical stalk domain, the 1B domain and the two RecA-like domains 1A and 2A, which contain the six conserved active site motifs. Thus, it is particularly preferred that infection with viruses comprising helicases belonging to the SF1 superfamily are treated with the compounds of the present invention.

Viral helicases are motor proteins that use energy derived from ATP hydrolysis to catalyze the unwinding of RNA or DNA duplex oligonucleotides into single strands in a 5′ to 3′ direction. This enzymatic activity is absolutely necessary for the viral genome replication and it has been shown to be required in transcription of viral mRNAs, translation, disruption of RNA-protein complexes, and packaging of nucleic acids into virions. Importantly, the validation of helicases as antiviral drug targets to reduce the viral replication has been demonstrated in animal models for the herpes simplex helicase (Crute, J. J., et al. (2002) Nat Med., 8:386-391 and Kleymann G, et al. (2002) Nat Med. 2002; 8:392-398).

However, the development of non-toxic helicase inhibitors has historically been considered much more challenging than developing drugs targeting other viral enzymes, as the helicase ATP-binding site is conserved not only in the different classes of helicases, but also in in motor proteins, small GTPases, kinases, the AAA+ family of ATPases, etc. (see FIG. 1A from D. N. Frick and A. M. I. Lam (2006) Curr. Pharm. Des., 12(11): 1315-1338). Thus, compounds that inhibit helicases via an ATP competitive mechanism have generally been regarded as potentially toxic. Another inherent problem of ATP competitive inhibitors of helicases is the high ATP concentration in target cells, which is typically maintained in the rage of 1 to 10 mmol/L. Effective inhibition by ATP competitive inhibitors either requires attaining high intracellular concentrations of the inhibitor or the identification of competitive inhibitors that bind to the NTPase active site with K_{D}s hat are several orders of magnitude lower than the binding of ATP to the NTPase active site or which covalently bind to the NTPase active site. Mirza M. U. & Froeyen M. (J. Pharm. Anal. (2020) 10(4):320-328) have elucidated the structure of three SARS-CoV-2 proteins by computational methods, namely the main protease, Nsp 12 polymerase and Nsp13 helicase. Using in silico methods they have identified potential binding modes and structural important binding site residues located in the ATP binding site and have identified potential drug candidates binding to the ATP binding site. Regarding Nsp13 helicase, they have identified several small molecules able to inhibit the NTPase activity by interferences with ATP binding in the active site, i.e. Mirza et al. (supra) have identified competitive inhibitors of the ATP binding site of Nsp13 helicase.

Ugurel and co-workers (O. M. Ugurel et al. Int. J. Biol. Macromol. (2020) 163:1687-1696) have evaluated the potency of FDA-approved drugs on wild-type and mutant SARS-CoV-2 helicase (Nsp13) using in silico methods. They state that the most potent drugs were found to interact with the key and neighbor residues of the active site responsible for ATP hydrolysis. However, Ugurel et al. (supra) did not actually test the activity of any of the compounds for their ability to inhibit Nsp13 helicase. Accordingly, Ugurel et al. have identified by in silico modeling potential competitive inhibitors of ATP binding to Nsp13 helicase.

Gurung A. B. (Gene Reports (2020) 21:100860) has also performed in silico structure modelling of SARS-CoV-2 Nsp13 helicase and Nsp14 and has further carried out a virtual screening (i.e. also in silico) of FDA approved antiviral drugs. To this end, the NTPase binding pocket (i.e. the active site) of Nsp13 helicase was used for docking the antiviral drugs. However, Gurung did not actually test the activity of any of the compounds for their ability to inhibit Nsp13 helicase. Accordingly, Ugurel et al. have identified by in silico modeling potential competitive inhibitors of ATP binding to Nsp13 helicase. Thus, there is a need in the art to identify novel inhibitors of viral helicases, in particular of Nsp13 helicases that inhibit helicase activity not by competitive inhibition of ATP binding but by mechanisms that are more specific to helicases and which are not as demanding regarding the K_{D}. The present invention overcomes these and other problems in the art by providing compounds inhibiting the enzymatic activity of viral helicases by binding to an allosteric regulation site of the viral helicase rather than to the ATP binding site itself. The compounds of the present invention inhibit the enzymatic activity in particular of the helicase of viruses of the class Pisoniviricetes, more preferably the helicase of viruses of the order Nidovirales, even more preferably the helicase of viruses of the suborder Cornidovirineae and most preferably the helicase of viruses of the family Coronaviridae. Within the family of Coronaviridae the non-structural protein 13 (Nsp13) helicase of SARS-CoV-2 is a particularly preferred target. The compounds of the present invention target a novel allosteric binding pocket of the viral helicase, preferably the Nsp13 helicase of SARS-CoV-2. This pocket was identified using molecular dynamics and subsequent docking of small molecules. The present invention further relates to such compounds for use in the prophylaxis and treatment of viral infections, in particular from the class of Pisoniviricetes, more preferably the order of Nidovirales, even more preferably the suborder of Cornidovirineae and most preferably of the family of Coronaviridae. Thus, present invention relates to prophylaxis and treatment of infections by all viral species that comprise a homologous helicase.

The compounds of the present invention provide inter alia the following advantages over the prior art: (i) they target a structural protein that is not only highly conserved among Coronaviridae (more than 99% amino acid sequence identity within this virus family) but also among viruses belonging to the class of Pisoniviricetes and, thus it is expected that infections with novel coronavirus that are either the result of mutations of the existing coronaviruses with pathogenicity in humans or that change from an animal host to humans can also be prevented and/or treated with the compounds of the present invention, (ii) they target an allosteric binding pocket of the viral helicase and thus it is plausible that the off-target effects are much lower than for compounds that may function via an ATP competitive mechanism, and/or (iii) the compounds are effective even when they are low affinity binders. Accordingly, the compounds of the present invention targeting these sites are very specific in their effect on viruses comprising helicases belonging to the SF1 superfamily.

### SUMMARY OF THE INVENTION

In a first aspect, the present invention is directed to an inhibitor of the helicase activity of a Nsp13 helicase of SARS-CoV-2 or of a viral homologue thereof, wherein the inhibitor specifically binds to an allosteric binding pocket within the N-terminal lobe of the ATPase domain of Nsp13 or of a viral homologue thereof.

In a second aspect, the present invention is directed to an inhibitor of the helicase activity of a Nsp13 helicase according to formula (I) to (IV).

In a third aspect, the present invention is directed to a pharmaceutical composition comprising the inhibitor according to the first or second aspects of the invention.

In a fourth aspect, the present invention is directed to the inhibitor according to the first or second aspect of the invention or the pharmaceutical composition of the third aspect of the invention for use in therapy.

In a fifth aspect, the present invention is directed to the inhibitor according to the first or second aspect of the invention or the pharmaceutical composition of the third aspect of the invention for use in prophylaxis or therapy of a viral infection.

In a preferred embodiment of the fifth aspect of the invention the viral infection is an infection with a positive stranded single stranded (ss) RNA.

### DESCRIPTION OF THE FIGURES

FIG. 1 Shows the sequence divergence between non-structural proteins of SARS-CoV and SARS-CoV-2 (FIG. 1 of Frick, D. N. et al (2020) Biochemistry, 59:2608-2615). The viral helicase is the most conserved protein among the NSPs with a sequence identity on amino acid level of >99%.

FIG. 2 Panel (A) shows the evolutionary relationship of viral helicases to other viral and cellular helicases (taken from Frick & Lam (2006) supra). Panel (B) shows the phylogenetic tree of the seven known human CoV helicases (B).

FIG. 3 Shows an alignment of the helicase that forms the allosteric binding pocket from the seven corona virus, namely Nsp13 helicase of HCoV-229E (SEQ ID NO: 2) referred to as “229E” in the figure, Nsp13 helicase of HCoV-NL63 (SEQ ID NO: 3) referred to as “NL-63” in the figure, Nsp13 helicase of HCoV OC43 (SEQ ID NO: 4) referred to as “OC43” in the figure, Nsp13 helicase of HCoV HKU1 (SEQ ID NO: 5) referred to as “HKU1” in the figure, Nsp13 helicase of MERS CoV (SEQ ID NO: 6), Nsp13 helicase of SARS CoV (SEQ ID NO: 7) and Nsp13 helicase SARS CoV2 (SEQ ID NO: 1). The allosteric binding pockets of the seven coronavirus helicases are highlighted by a box. In SARS CoV2 the allosteric binding pocket spans 5236-T440. Out of those 205 amino acids only 28 form the binding pocket. These amino acids are highlighted by bold print in SARS-CoV2 and also in the six other coronavirus helicases. Within the 28 amino acids forming the binding pocket 57.1% of the amino acid residues are identical, 17.9% are highly similar, 10.7% similar, and 14.3% are different. Accordingly, within the region relevant for the interaction of the compounds of the present invention the seven coronavirus helicases have a homology of 85.7%. Furthermore, the alignment led to the identification of several conserved motifs with the coronavirus helicases, which are highlighted in all seven amino acid sequences by underline. Furthermore, amino acids highlighted in light grey are absolutely conserved in the active site motifs.

FIG. 4 Panel (A) shows a cartoon representation of the structure of the SARS-CoV2 Nsp13 (taken from PDB 6XEZ, Chen et al., 2020, Cell 182, 1-14). The N-terminal zinc binding domain (ZBD) with three bound zinc ions (dark spheres), the stalk domain, the 1B domain and the two RecA-like domains 1A and 2A are labelled accordingly. The nucleotide binding cleft with the bound transition state analog ADP-AF₃ is indicated by an arrow. Panel (B) highlights the RecA lobes 1A and 2A, the conserved residues from the six active site motifs are shown as sticks. The transition state analog ADP-AF₃ (grey) and the co-factor Mg²⁺ (black) are shown as spheres. The location of the newly identified allosteric pocket is indicated by the surface representation and does not overlap with the nucleotide binding site.

FIG. 5 Panel (A) shows the top view of the sliced surface of SARS-CoV2 Helicase domain N-Terminal Lobe S236-T440 (Lobe 1) with its tripartite pocket consisting of entry channel (1), left channel (2), and right channel (3), and active site (4) circled. Panel (B) shows the bottom view of the sliced surface of SARS-CoV2 Helicase domain N-Terminal Lobe S236-T440 (Lobe 1) with its tripartite pocket consisting of entry channel (1), left channel (2), and right channel (3), and active site (4) circled.

FIG. 6 A) Top view of the sliced surface of SARS-CoV-2 helicase domain Lobe 1A with colored by hydrophobicity with hydrophilic regions shown in white and hydrophobic regions shown in black. B) Bottom view of the sliced surface of SARS-CoV-2 helicase domain Lobe 1A colored by hydrophobicity with hydrophilic regions shown in white and hydrophobic regions shown in black.

FIG. 7 A) Top view of the sliced surface of SARS-CoV-2 helicase domain Lobe 1A with compound COVI-3 addressing the entry channel and right channel. B) Bottom view of the sliced surface of SARS-CoV-2 helicase domain Lobe 1A with compound COVID Inhibitor (COVI) 3 (COVI-3) addressing the entry channel and right channel. C) Top view of the sliced surface of SARS-CoV2 Helicase domain Lobe 1 with compound COVI-10 addressing the entry channel, right channel, and left channel. D) Bottom view of the sliced surface of SARS-CoV2 helicase domain Lobe 1A with compound COVI-10 addressing the entry channel, right channel, and left channel. E) Top view of the sliced surface of SARS-CoV2 Helicase domain Lobe 1 with compound COVI-35 addressing the entry channel and right channel. F) Bottom view of the sliced surface of SARS-CoV2 helicase domain Lobe 1A with compound COVI-35 addressing the entry channel and right channel.

FIG. 8 Panel (A) shows the molecular surface of compound COVI-10 shown from three different angles. Region 1 contains polar substituents while regions 2 and 3 are largely unpolar. Panel (B) The molecular surface of compound COVI-3 shown from three different angles. Region 1 contains polar substituents while regions 2 and 3 are largely unpolar. (C) The molecular surface of compound COVI-35 shown from three different angles. Region 1 contains polar substituents while region 2 is unpolar.

FIG. 9 Panel (A) shows top view of the surface of compound COVI-10 surrounded by the residues making up the binding pocket of the SARS-CoV2 helicase domain lobe 1A. Panel (B) Bottom view of the surface of compound COVI-10 surrounded by the residues making up the binding pocket of the SARS-CoV-2 Helicase domain lobe 1. Panel (C) Top view of the surface of compound COVI-3 surrounded by the residues making up the binding pocket of the SARS-CoV2 Helicase domain lobe 1. Panel (D) Bottom view of the surface of compound COVI-3 surrounded by the residues making up the binding pocket of the SARS-CoV2 Helicase domain lobe 1. Panel (E) Top view of the surface of compound COVI-35 surrounded by the residues making up the binding pocket of the SARS-CoV2 Helicase domain lobe 1. Panel (F) Bottom view of the surface of compound COVI-35 surrounded by the residues making up the binding pocket of the SARS-CoV2 Helicase domain lobe 1.

FIG. 10 Panel (A) shows the bottom view of compound COVI-10 showing specific interactions with N158, F143, and 1169. B) Bottom view of compound COVI-3 showing specific interactions with N158. C) Top view of compound COVI-35 showing specific interactions with M429.

FIG. 11 Panel (A) shows a ligplot diagram of compound COVI-10 in the allosteric binding site of SARS-CoV2 Nsp13 lobe 1. Panel (B) shows a ligplot diagram of compound COVI-3 in the allosteric binding site of SARS-CoV2 Nsp13 lobe 1. Panel (C) shows a ligplot diagram of compound COVI-35 in the allosteric binding site of SARS-CoV2 Nsp13 lobe 1.

FIG. 12 Shows dose response curves of the active SARS-CoV-2 Nsp13 inhibitors tested in a FRET-based DNA unwinding helicase assay.

FIG. 13 Shows a dose response curves of selected SARS-CoV2 Nsp13 inhibitors tested in a malachite green based ATPase assay.

FIG. 14 Table indicating the structures of the COVI inhibitors and the respective IC₅₀ determined in the DNA unwinding and ATPase assays.

FIG. 15 Shows dose response curves of selected Nsp13 inhibitors tested in a cellular infection assay with the 229E coronavirus.

FIG. 16 Shows dose response curves of selected SARS-CoV2 Nsp13 inhibitors tested in SARS-CoV-2 nanoluciferase assay.

FIG. 17 A general synthesis scheme for compounds of formula (I) comprising a pyrazole core structure. The three substituents which are subsequently covalently coupled to the core are shown without substituents but can either comprise such substituents when coupled and/or can comprise protection groups that protect such substituents during the coupling reaction and which are subsequently removed or they can comprise protection groups that can subsequently be cleaved to create new reactive groups to which the desired substituent is ultimately coupled as commonly known in the art.

FIG. 18 A general synthesis scheme for compounds of formula (II) comprising a quinazoline core structure. The two substituents which are subsequently covalently coupled to the core are shown without substituents but can either comprise such substituents when coupled and/or can comprise protection groups that protect such substituents during the coupling reaction and which are subsequently removed or they can comprise protection groups that can subsequently be cleaved to create new reactive groups to which the desired substituent is ultimately coupled as commonly known in the art.

FIG. 19 A general synthesis scheme for compounds of formula (III)

FIG. 20 A list of suppliers from which each compound was obtained.

FIG. 21 Panel (A) Shows the sliced surface of SARS-CoV-2 Nsp13 helicase domain Lobe 1A with compound COVI-3 addressing the entry channel and right channel. The binding site of inhibitors reported by Mirza and Froeyen (supra) and those reported by Gurung (supra) is circled and residues shared between the binding sites (D144, E145, and M148) are colored black. Panel (B) shows a ribbon representation of SARS-CoV-2 Nsp13 helicase domain Lobe 1A with the amino acids directly interacting with the compounds reported by Mirza and Froeyen and those reported by Gurung in black, the binding site residues directly interacting with the presently reported compounds in light grey, and those shared in white. It is evident that the inhibitors reported by Mirza and Froeyen (supra) and those reported by Gurung (supra) bind to a different binding pocket of NSP13 and, thus that the inhibitors of the prior art differ from the compounds of the present invention that bind to an allosteric binding pocket of viral helicases.

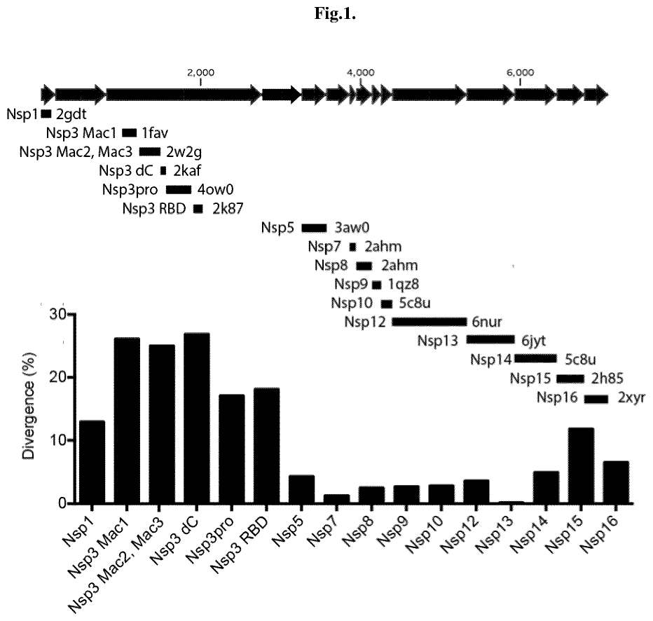

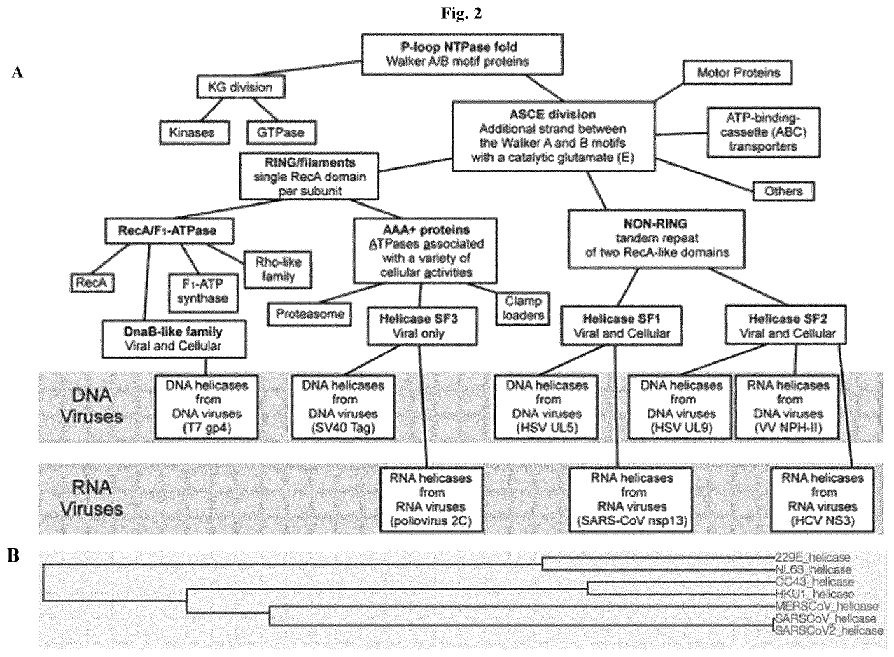

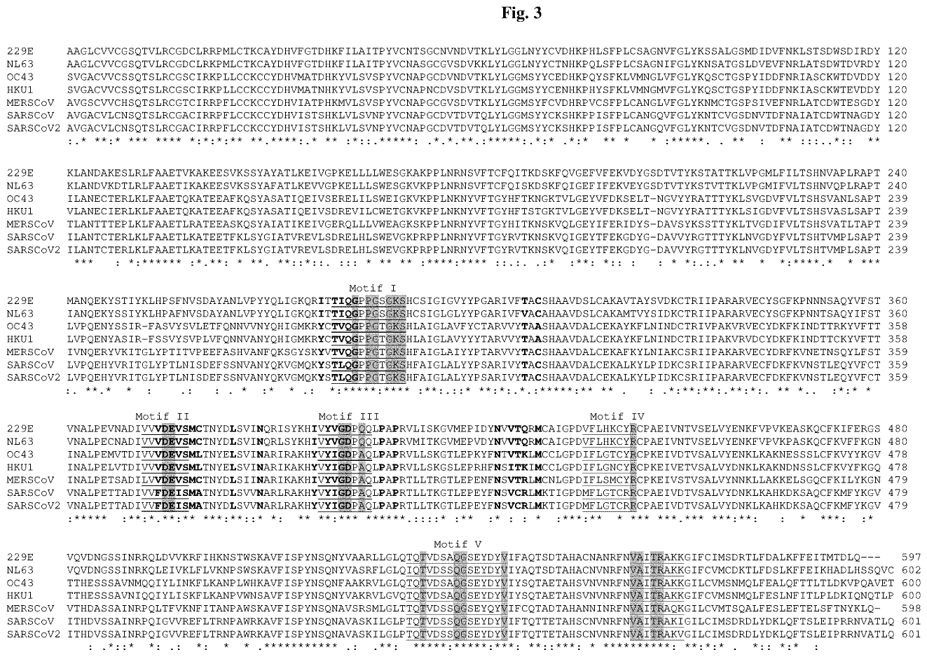

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

### Definitions

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. As used in this specification and the appended claims, the singular forms “a”, “an”, and “the” include plural referents, unless the content clearly dictates otherwise.

In the following paragraphs, definitions of the terms: alkyl, heteroalkyl, haloalkyl, cycloalkyl, heterocycloalkyl, aryl, aralkyl, heteroaryl, heteroaralkyl, alkenyl, cycloalkenyl, heteroalkenyl, heterocycloalkenyl, and alkynyl are provided. These terms will in each instance of its use in the remainder of the specification have the respectively defined meaning and preferred meanings. Nevertheless, in some instances of their use throughout the specification preferred meanings of these terms are indicated.

The term “alkyl” refers to a saturated straight or branched carbon chain. Preferably, the chain comprises from 1 to 10 carbon atoms, i.e. 1, 2, 3, 4, 5, 6, 7, 8, 9 or 10, e.g. methyl, ethyl propyl (n-propyl or iso-propyl), butyl (n-butyl, iso-butyl, sec-butyl, tert-butyl), pentyl, hexyl, heptyl, octyl, nonyl, decyl. Alkyl groups are optionally substituted.

The term “heteroalkyl” refers to a saturated straight or branched carbon chain. Preferably, the chain comprises from 1 to 9 carbon atoms, i.e. 1, 2, 3, 4, 5, 6, 7, 8, or 9, e.g. methyl, ethyl, propyl, iso-propyl, butyl, iso-butyl, sec-butyl, tert-butyl, pentyl, hexyl, heptyl, octyl, nonyl, which is interrupted one or more times, e.g. 1, 2, 3, 4, 5, with the same or different heteroatoms. Preferably, the heteroatoms are selected from O, S, and N, e.g. —(CH₂)ₙ—X—(CH₂)ₘCH₃, with n=0, 1, 2, 3, 4, 5, 6, 7, 8, or 9, m=0, 1, 2, 3, 4, 5, 6, 7, 8, or 9 and X=S, O or NR′ with R′=H or hydrocarbon (e.g. C₁ to C₆ alkyl). In particular, “heteroalkyl” refers to —O—CH₃, —OC₂H₅, —CH₂—O—CH₃, —CH₂—O—C₂H₅, —CH₂—O—C₃H₇, —CH₂—O—C₄H₉, —CH₂—O—C₅H₁₁, —C₂H₄O—CH₃, —C₂H₄—O—C₂H₅, —C₂H₄—O—C₃H₇, —C₂H₄—O—C₄H₉ etc. Heteroalkyl groups are optionally substituted.

The term “haloalkyl” refers to a saturated straight or branched carbon chain in which one or more hydrogen atoms are replaced by halogen atoms, e.g. by fluorine, chlorine, bromine or iodine. Preferably, the chain comprises from 1 to 10 carbon atoms, i.e. 1, 2, 3, 4, 5, 6, 7, 8, 9 or 10. In particular, “haloalkyl” refers to —CH₂F, —CHF₂, —CF₃, —C₂H₄F, —C₂H₃F₂, —C₂H₂F₃, —C₂HF₄, —C₂F₅, —C₃H₆F, —C₃H₅F₂, —C₃H₄F₃, —C₃H₃F₄, —C₃H₂F₅, —C₃HF₆, —C₃F₇, —CH₂Cl, —CHCl₂, —CCl₃, —C₂H₄Cl, —C₂H₃Cl₂, —C₂H₂Cl₃, —C₂HCl₄, —C₂Cl₅, —C₃H₆Cl, —C₃H₅Cl₂, —C₃H₄Cl₃, —C₃H₃Cl₄, —C₃H₂Cl₅, —C₃HCl₆, and —C₃Cl₇. Haloalkyl groups are optionally substituted.

The terms “cycloalkyl” and “heterocycloalkyl” alone or in combination with other terms, represent, unless otherwise stated, cyclic versions of “alkyl” and “heteroalkyl”, respectively, with preferably 3, 4, 5, 6, 7, 8, 9 or 10 atoms forming a ring, e.g. cyclopropyl, cyclobutyl, cyclopentyl, cyclohexyl, cycloheptyl, cyclooctyl etc. The terms “cycloalkyl” and “heterocycloalkyl” are also meant to include bicyclic, tricyclic and polycyclic versions thereof. If bicyclic, tricyclic or polycyclic rings are formed, it is preferred that the respective rings are connected to each other at two adjacent carbon atoms, however, alternatively the two rings are connected via the same carbon atom, i.e. they form a spiro ring system or they form “bridged” ring systems, preferably tricycle[3.3.1.1^{3,7}]decan. The term “heterocycloalkyl” preferably refers to a saturated ring having five members of which at least one member is an N, O or S atom and which optionally contains one additional O or one additional N; a saturated ring having six members of which at least one member is an N, O or S atom and which optionally contains one additional O or one additional N or two additional N atoms; or a saturated bicyclic ring having nine or ten members of which at least one member is an N, O or S atom and which optionally contains one, two or three additional N atoms. “Cycloalkyl” and “heterocycloalkyl” groups are optionally substituted. Additionally, for heterocycloalkyl, a heteroatom can occupy the position at which the heterocycle is attached to the remainder of the molecule. Examples of cycloalkyl include cyclopropyl, cyclobutyl, cyclopentyl, cyclohexyl, cycloheptyl, spiro[3,3]heptyl, spiro[3,4]octyl, spiro[4,3]octyl, spiro[3,5]nonyl, spiro[5,3]nonyl, spiro[3,6]decyl, spiro[6,3]decyl, spiro[4,5]decyl, spiro[5,4]decyl, bicyclo[2.2.1]heptyl, bicyclo[2.2.2]octyl, adamantyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, 1,8-diazo-spiro[4,5]decyl, 1,7-diazo-spiro[4,5]decyl, 1,6-diazo-spiro[4,5]decyl, 2,8-diazo-spiro[4,5]decyl, 2,7-diazo-spiro[4,5]decyl, 2,6-diazo-spiro[4,5]decyl, 1,8-diazo-spiro[5,4]decyl, 1,7 diazo-spiro[5,4]decyl, 2,8-diazo-spiro[5,4]decyl, 2,7-diazo-spiro[5,4]decyl, 3,8-diazo-spiro[5,4]decyl, 3,7-diazo-spiro[5,4]decyl, 1,4-diazabicyclo[2.2.2]oct-2-yl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” preferably refers to an aromatic monocyclic ring containing 6 carbon atoms, an aromatic bicyclic ring system containing 10 carbon atoms or an aromatic tricyclic ring system containing 14 carbon atoms. Examples are phenyl, naphthyl or anthracenyl. The aryl group is optionally substituted.

The term “aralkyl” refers to an alkyl moiety, which is substituted by aryl, wherein alkyl and aryl have the meaning as outlined above. An example is the benzyl radical. Preferably, in this context the alkyl chain comprises from 1 to 8 carbon atoms, i.e. 1, 2, 3, 4, 5, 6, 7, or 8, e.g. methyl, ethyl, propyl, iso-propyl, butyl, iso-butyl, sec-butyl, tert-butyl, pentyl, hexyl, heptyl, octyl. The aralkyl group is optionally substituted at the alkyl and/or aryl part of the group. Preferably the aryl attached to the alkyl has the meaning phenyl, naphthyl or anthracenyl.

The term “heteroaryl” preferably refers to a five or six-membered aromatic monocyclic ring wherein at least one of the carbon atoms is replaced by 1, 2, 3, or 4 (for the five membered ring) or 1, 2, 3, 4, or 5 (for the six membered ring) of the same or different heteroatoms, preferably selected from O, N and S; an aromatic bicyclic ring system with 8 to 12 members wherein 1, 2, 3, 4, 5, or 6 carbon atoms of the 8, 9, 10, 11 or 12 carbon atoms have been replaced with the same or different heteroatoms, preferably selected from O, N and S; or an aromatic tricyclic ring system with 13 to 16 members wherein 1, 2, 3, 4, 5, or 6 carbon atoms of the 13, 14, 15, or 16 carbon atoms have been replaced with the same or different heteroatoms, preferably selected from O, N and S. Examples are furanyl, thiophenyl, oxazolyl, isoxazolyl, 1,2,4-oxadiazolyl, 1,2,3-oxadiazolyl, pyrrolyl, imidazolyl, pyrazolyl, 1,2,3-triazolyl, 1,2,4-triazolyl, thiazolyl, isothiazolyl, 1,2,3-thiadiazolyl, 1,2,4-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2,1-benzosoxazoyl, benzothiazolyl, 1,2-benzisothiazolyl, 2,1-benzisothiazolyl, benzotriazolyl, quinolinyl, isoquinolinyl, 2,3-benzodiazinyl, quinoxalinyl, quinazolinyl, quinolinyl, 1,2,3-benzotriazinyl, or 1,2,4-benzotriazinyl. The heteroaryl group may be optionally substituted.

The term “heteroaralkyl” refers to an alkyl moiety, which is substituted by heteroaryl, wherein alkyl and heteroaryl have the meaning as outlined above. An example is the 2-alkylpyridinyl, 3-alkylpyridinyl, or 2-methylpyridinyl radical. Preferably, in this context the alkyl chain comprises from 1 to 8 carbon atoms, i.e. 1, 2, 3, 4, 5, 6, 7, or 8, e.g. methyl, ethyl, propyl, iso-propyl, butyl, iso-butyl, sec-butyl, tert-butyl, pentyl, hexyl, heptyl, octyl. The heteroaralkyl group is optionally substituted at the alkyl and/or heteroaryl part of the group. Preferably the heteroaryl attached to the alkyl has the meaning oxazolyl, isoxazolyl, 1,2,5-oxadiazolyl, 1,2,3-oxadiazolyl, pyrrolyl, imidazolyl, pyrazolyl, 1,2,3-triazolyl, 1,2,4-triazolyl, thiazolyl, isothiazolyl, 1,2,3-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2,1-benzosoxazoyl, benzothiazolyl, 1,2-benzisothiazolyl, 2,1-benzisothiazolyl, benzotriazolyl, 2,3-benzodiazinyl, quinolinyl, isoquinolinyl, quinoxalinyl, quinazolinyl, quinolinyl, 1,2,3-benzotriazinyl, or 1,2,4-benzotriazinyl. The heteroaralkyl group may be optionally substituted at the alkyl and/or heteroaryl part of the group.

The terms “alkenyl” and “cycloalkenyl” refer to olefinic unsaturated carbon atoms containing chains or rings with one or more double bonds. Examples are propenyl and cyclohexenyl. Preferably, the alkenyl chain comprises from 2 to 8 carbon atoms, i.e. 2, 3, 4, 5, 6, 7, or 8, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl, octenyl. Preferably the cycloalkenyl ring comprises from 3 to 8 carbon atoms, i.e. 3, 4, 5, 6, 7, or 8, e.g. 1-cyclopropenyl, 2-cyclopropenyl, 1-cyclobutenyl, 2-cyclobutenyl, 1-cyclopentenyl, 2-cyclopentenyl, 3-cyclopentenyl, 1-cyclohexenyl, 2-cyclohexenyl, 3-cyclohexenyl, cycloheptenyl, cyclooctenyl. The “alkenyl” and “cycloalkenyl” groups may be optionally substituted.

The terms “heteroalkenyl” and “heterocycloalkenyl” refer to unsaturated versions of “heteroalkyl” and “heterocycloalkyl”, respectively. Thus, the term “heteroalkenyl” refers to an unsaturated straight or branched carbon chain. Preferably, the chain comprises from 1 to 9 carbon atoms, i.e. 1, 2, 3, 4, 5, 6, 7, 8, 9, which is interrupted one or more times, e.g. 1, 2, 3, 4, 5, with the same or different heteroatoms.

Preferably, the heteroatoms are selected from O, S, and N. In case that one or more of the interrupting heteroatoms is N, the N may be present as an —NR′— moiety, wherein R′ is hydrogen or hydrocarbon (e.g. C₁ to C₆ alkyl), or it may be present as an ═N— or —N═ group, i.e. the nitrogen atom can form a double bond to an adjacent C atom or to an adjacent, further N atom. “Heteroalkenyl” groups are optionally substituted.

The term “heterocycloalkenyl” represents a cyclic version of “heteroalkenyl” with preferably 3, 4, 5, 6, 7, 8, 9 or 10 atoms forming a ring. The term “heterocycloalkenyl” is also meant to include bicyclic, tricyclic and polycyclic versions thereof. If bicyclic, tricyclic or polycyclic rings are formed, it is preferred that the respective rings are connected to each other at two adjacent atoms. These two adjacent atoms can both be carbon atoms; or one atom can be a carbon atom and the other one can be a heteroatom; or the two adjacent atoms can both be heteroatoms. However, alternatively the two rings are connected via the same carbon atom, i.e. they form a spiro ring system or they form “bridged” ring systems. The term “heterocycloalkenyl” preferably refers to an unsaturated ring having five members of which at least one member is an N, O or S atom and which optionally contains one additional O or one additional N; an unsaturated ring having six members of which at least one member is an N, O or S atom and which optionally contains one additional O or one additional N or two additional N atoms; or an unsaturated bicyclic ring having nine or ten members of which at least one member is an N, O or S atom and which optionally contains one, two or three additional N atoms. “Heterocycloalkenyl” groups are optionally substituted. Additionally, for heteroalkenyl and heterocycloalkenyl, a heteroatom can occupy the position at which the heterocycle is attached to the remainder of the molecule.

The term “aralkenyl” refers to an alkenyl moiety, which is substituted by aryl, wherein alkenyl and aryl have the meaning as outlined above.

The term “heteroaralkenyl” refers to an alkenyl moiety, which is substituted by heteroaryl, wherein alkenyl and heteroaryl have the meaning as outlined above.

The term “alkynyl” refers to unsaturated carbon atoms containing chains or rings with one or more triple bonds. Preferably, the alkynyl chain comprises from 2 to 8 carbon atoms, i.e. 2, 3, 4, 5, 6, 7, or 8, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, heptynyl, octynyl. The alkynyl group may be optionally substituted.

The terms “heteroalkynyl”, “cycloalkynyl”, and “heterocycloalkynyl” refer to moieties that basically correspond to “heteroalkenyl”, “cycloalkenyl”, and “heterocycloalkenyl”, respectively, as defined above but differ from “heteroalkenyl”, “cycloalkenyl”, and “heterocycloalkenyl” in that at least one double bond is replaced by a triple bond.

As used herein, the term “alicyclic system” includes cycloalkyl, cycloalkenyl, and cycloalkynyl substituents, as defined above.

As used herein, the term “carbocycle” includes monocyclic cycloalkyl, cycloalkenyl, cycloalkynyl and aryl substituents.

As used herein, the term “heterocycle” includes monocyclic heterocycloalkyl, heterocycloalkenyl, heterocycloalkynyl and heteroaryl substituents.

In one embodiment, carbon atoms or hydrogen atoms in alkyl, cycloalkyl, aryl, aralkyl, alkenyl, cycloalkenyl, alkynyl radicals may be substituted independently from each other with one or more elements selected from the group consisting of O, S, N or with groups containing one or more elements, i.e. 1, 2, 3, 4, 5, 6, or more selected from the group consisting of O, S, and N.

Embodiments include alkoxy, cycloalkoxy, aryloxy, aralkoxy, alkenyloxy, cycloalkenyloxy, alkynyloxy, alkylthio, cycloalkylthio, arylthio, aralkylthio, alkenylthio, cycloalkenylthio, alkynylthio, alkylamino, cycloalkylamino, arylamino, aralkylamino, alkenylamino, cycloalkenylamino, alkynylamino radicals.

Other embodiments include hydroxyalkyl, hydroxycycloalkyl, hydroxyaryl, hydroxyaralkyl, hydroxyalkenyl, hydroxycycloalkenyl, hydroxyalkynyl, mercaptoalkyl, mercaptocycloalkyl, mercaptoaryl, mercaptoaralkyl, mercaptoalkenyl, mercaptocycloalkenyl, mercaptoalkynyl, aminoalkyl, aminocycloalkyl, aminoaryl, aminoaralkyl, aminoalkenyl, aminocycloalkenyl, aminoalkynyl radicals.

In another embodiment, one or more hydrogen atoms, e.g. 1, 2, 3, 4, 5, 6, 7, or 8 hydrogen atoms in alkyl, heteroalkyl, cycloalkyl, heterocycloalkyl, alicyclic system, aryl, aralkyl, heteroaryl, heteroaralkyl, alkenyl, cycloalkenyl, heteroalkenyl, heterocycloalkenyl, alkynyl radicals may be substituted independently from each other with one or more halogen atoms, e.g. Cl, F, or Br. One preferred radical is the trifluoromethyl radical.

If two or more radicals can be selected independently from each other, then the term “independently” means that the radicals may be the same or may be different.

The term “optionally substituted” in each instance if not further specified refers to halogen (in particular F, Cl, Br, or I), —NO₂, —CN, —OR′″, —NR′R″, —COOR′″, —CONR′R″, —NR′COR″, —NR″COR′″, —NR′CONR′R″, —NR′SO₂E, —COR′″; —SO₂NR′R″, —OOCR′″, −CR′″R″″OH, —R′″ OH, and -E;
R′ and R″ is each independently selected from the group consisting of hydrogen, alkyl, alkenyl, alkynyl, cycloalkyl, heterocycloalkyl, aryl, aralkyl, and heteroaryl or together form a heteroaryl, or heterocycloalkyl; R′″ and R″″ is each independently selected from the group consisting of hydrogen, alkyl, alkenyl, alkynyl, cycloalkyl, heterocycloalkyl, alkoxy, aryl, aralkyl, heteroaryl, and —NR′R″;
E is selected from the group consisting of alkyl, alkenyl, alkynyl, cycloalkyl, alkoxy, alkoxyalkyl, heterocycloalkyl, an alicyclic system, aryl and heteroaryl; optionally substituted.

As used in this specification the term “amino acid sequence identity” relates to the percentage of sequences identity and is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used in this specification the term “identical” in the context of two or more nucleic acids or polypeptide sequences, refers to two or more sequences or subsequences that are the same, i.e. comprise the same sequence of nucleotides or amino acids. Sequences are “substantially identical” to each other if they have a specified percentage of nucleotides or amino acid residues that are the same (e.g., at least 50%, at least 55%, at least 60%, at least 65%, at least 70%, at least 75%, at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity over a specified region), when compared and aligned for maximum correspondence over a comparison window, or designated region as measured using one of the following sequence comparison algorithms or by manual alignment and visual inspection. These definitions also refer to the complement of a test sequence. Accordingly, the term “at least 80% sequence identity” is used throughout the specification with regard to polypeptide and polynucleotide sequence comparisons. This expression preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

As used in this specification the term “sequence comparison” refers to the process wherein one sequence acts as a reference sequence, to which test sequences are compared. When using a sequence comparison algorithm, test and reference sequences are entered into a computer, if necessary subsequence coordinates are designated, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise.

In a sequence alignment, the term “comparison window” refers to those stretches of contiguous positions of a sequence which are compared to a reference stretch of contiguous positions of a sequence having the same number of positions. The number of contiguous positions selected may range from 10 to 1000, i.e. may comprise 10, 20, 30, 40, 50, 60, 70, 80, 90, 100, 150, 200, 250, 300, 350, 400, 450, 500, 550, 600, 650, 700, 750, 800, 850, 900, 950, or 1000 contiguous positions. Typically, the number of contiguous positions ranges from about 20 to 800 contiguous positions, from about 20 to 600 contiguous positions, from about 50 to 400 contiguous positions, from about 50 to about 200 contiguous positions, from about 100 to about 150 contiguous positions.

Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

Semi-conservative and especially conservative amino acid substitutions, wherein an amino acid is substituted with a chemically related amino acid are preferred. Typical substitutions are among the aliphatic amino acids, among the amino acids having aliphatic hydroxyl side chain, among the amino acids having acidic residues, among the amide derivatives, among the amino acids with basic residues, or the amino acids having aromatic residues. Typical semi-conservative and conservative substitutions are:

| | Conservative | Semi-conservative |
|---|---|---|
| Amino acid | substitution | substitution |
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C. |

“Pharmaceutically acceptable” means approved by a regulatory agency of the Federal or a state government or listed in the U.S. Pharmacopeia (United States Pharmacopeia-33/National Formulary-28 Reissue, published by the United States Pharmacopeia Convention, Inc., Rockville Md., publication date: April 2010) or other generally recognized pharmacopeia for use in animals, and more particularly in humans.

The term “pharmaceutically acceptable salt” refers to a salt of a compound of the present invention.

Suitable pharmaceutically acceptable salts of the compound of the present invention include acid addition salts which may, for example, be formed by mixing a solution of a compound described herein or a derivative thereof with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxyethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are isomers of the compounds defined by general formulae (I), (II), (III), and (IV). The term “isomers” comprises structural isomers and stereoisomers, including enantiomers, diastereomers, cis-trans isomers, conformers, and rotamers. In the context of the present invention, it is preferred that “isomers” are stereoisomers (and not structural isomers).

The present invention also provides solvates of the compounds defined by general formulae (I), (II), (III), and (IV). The term “solvate” according to the invention is to be understood as meaning any form of the compounds which has another molecule (for example a polar solvent) attached to it via non-covalent bonding. Examples of solvates include hydrates and alcoholates, e.g. methanolates and ethanolates. Solvation methods are generally known in the state of the art.

The present invention further provides chemically protected forms of the compounds defined by general formulae (I), (II), (III), and (IV). During any of the processes for preparation of the compounds of the present invention, it may be necessary and/or desirable to protect sensitive or reactive groups on any of the molecules concerned, thereby resulting in chemically protected forms of the compounds of the present invention. This may be achieved by means of conventional protecting groups. The protecting groups may be removed at a convenient subsequent stage using methods known in the art.

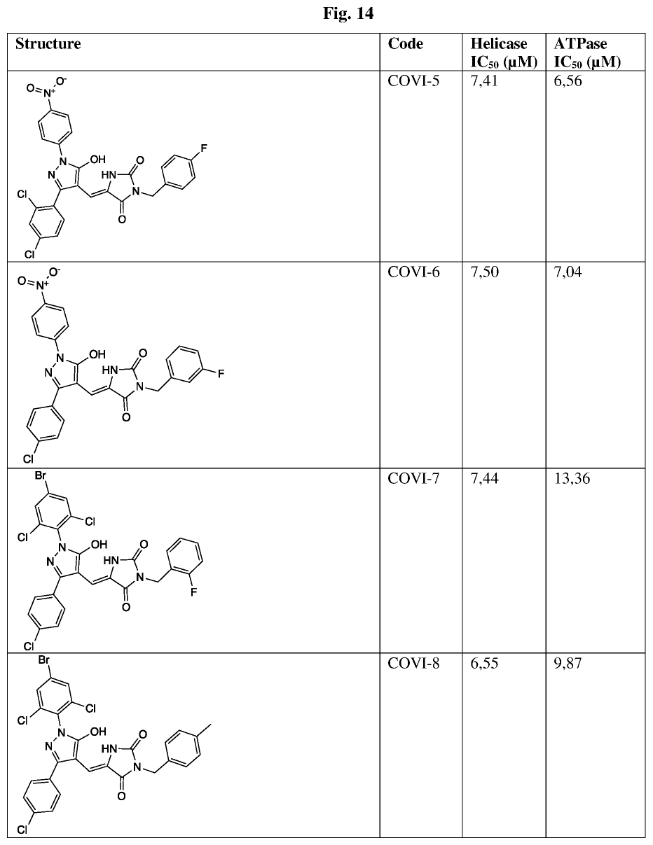

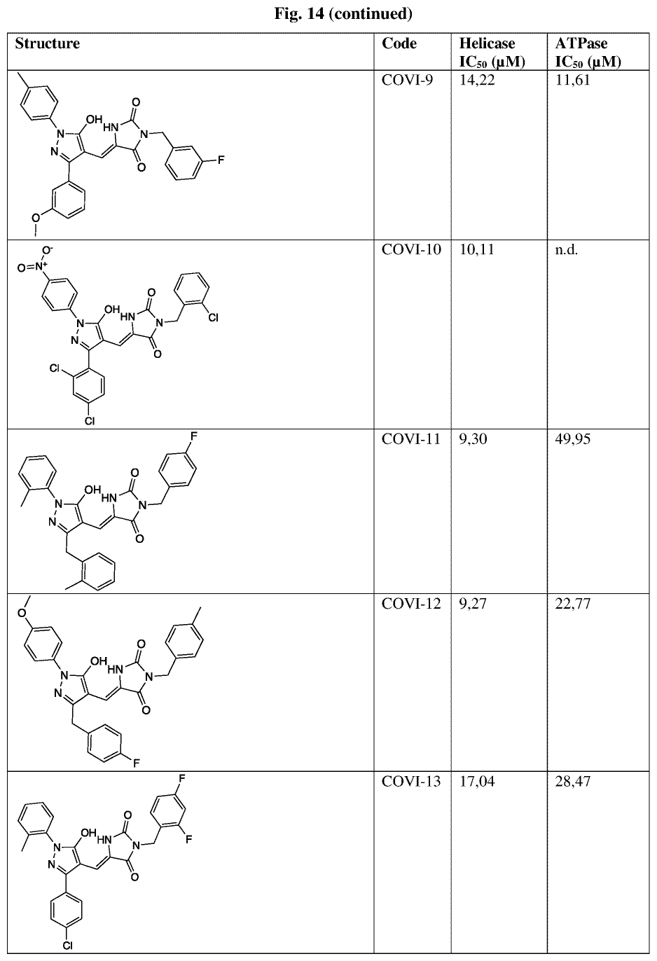

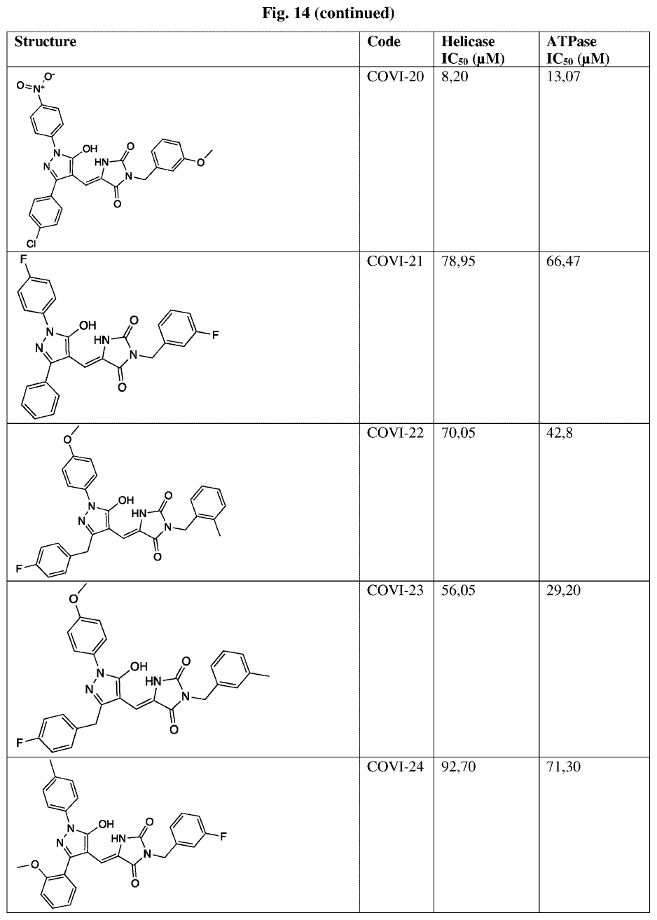

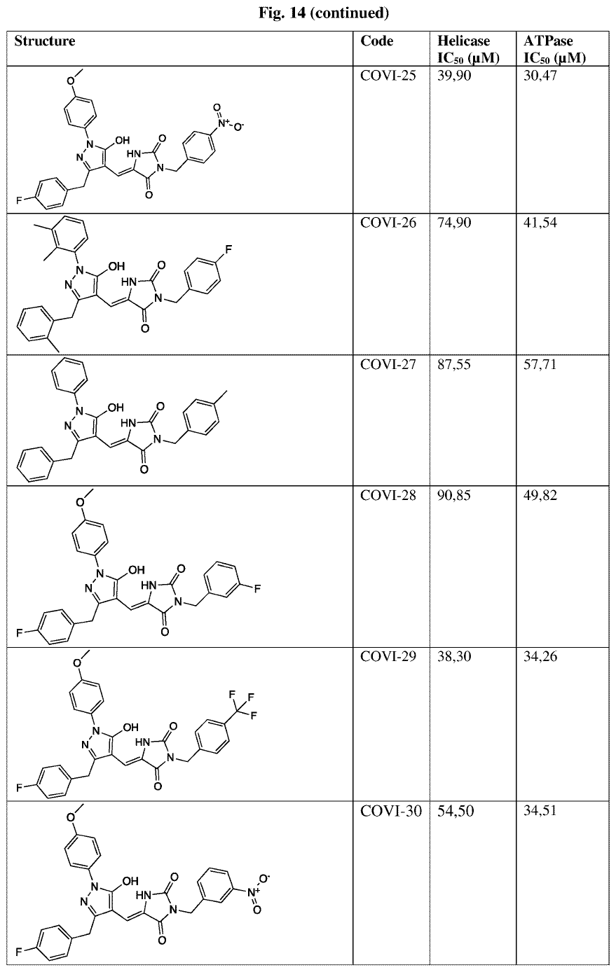

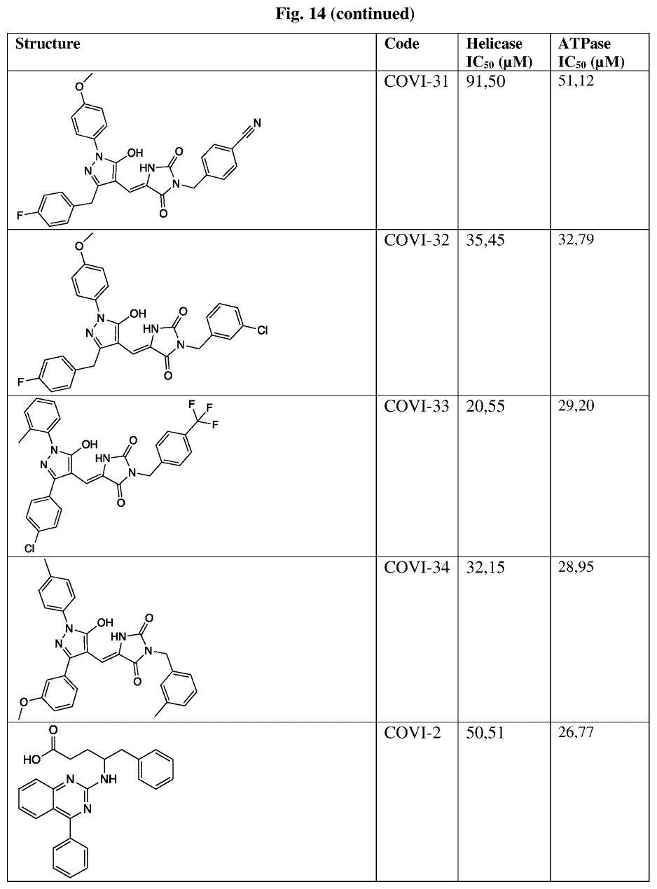

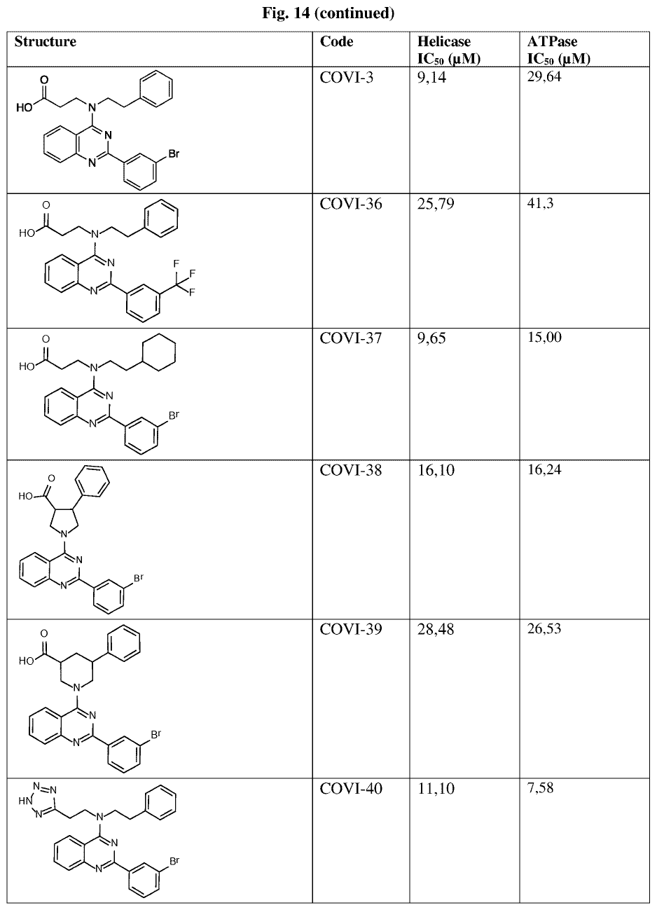

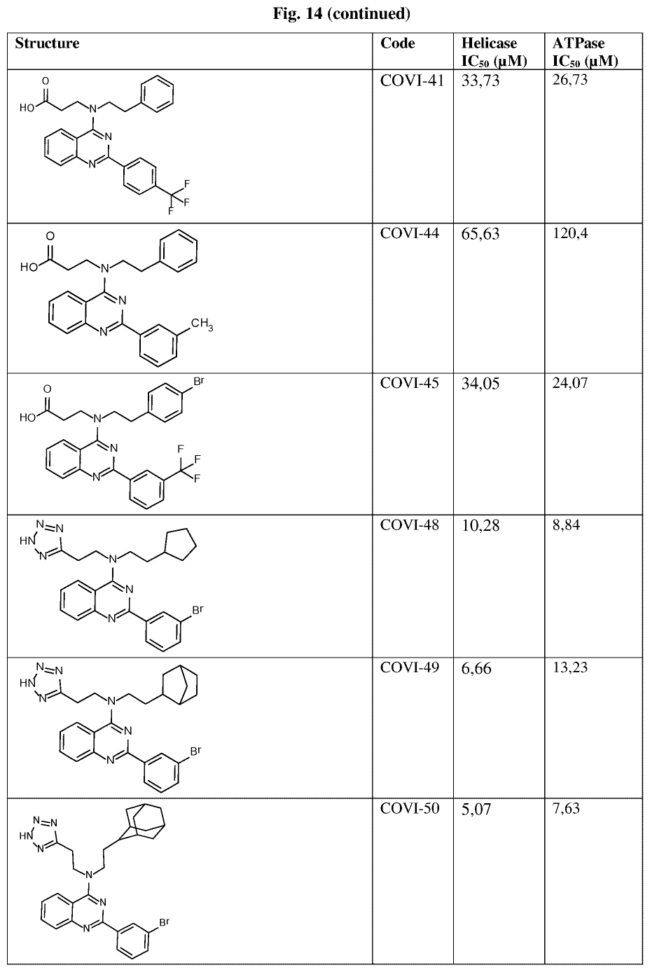

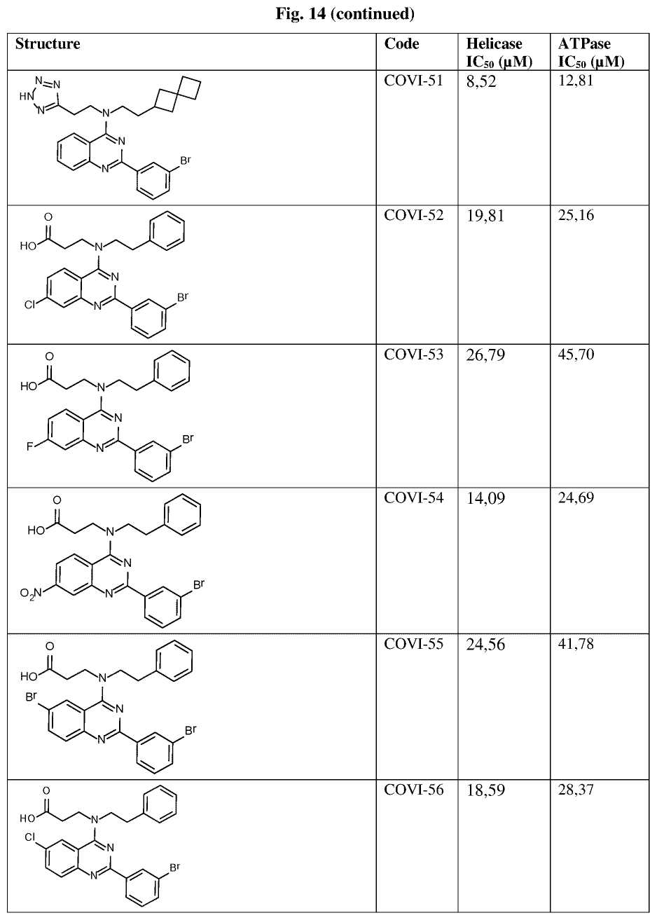

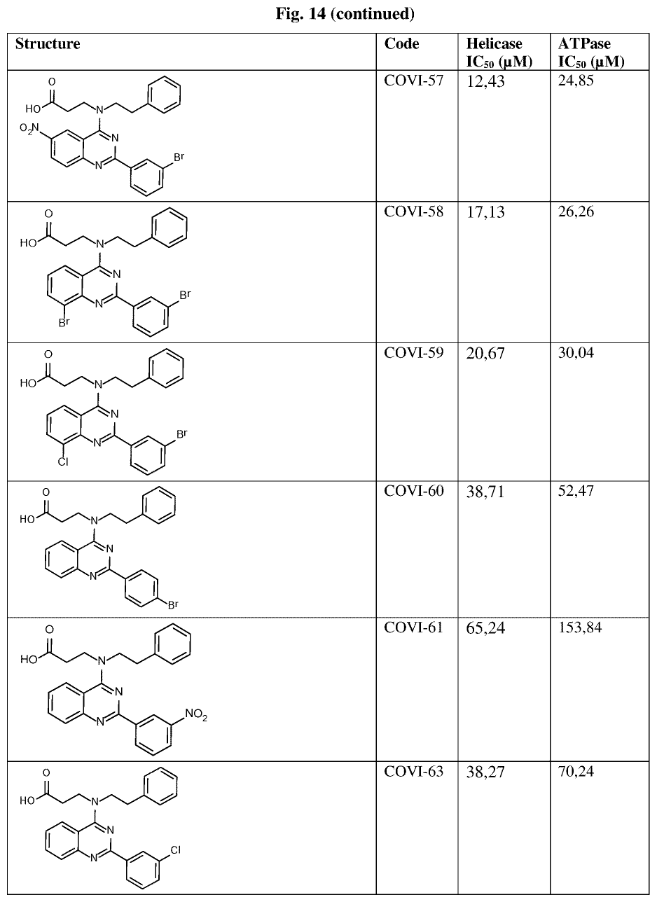

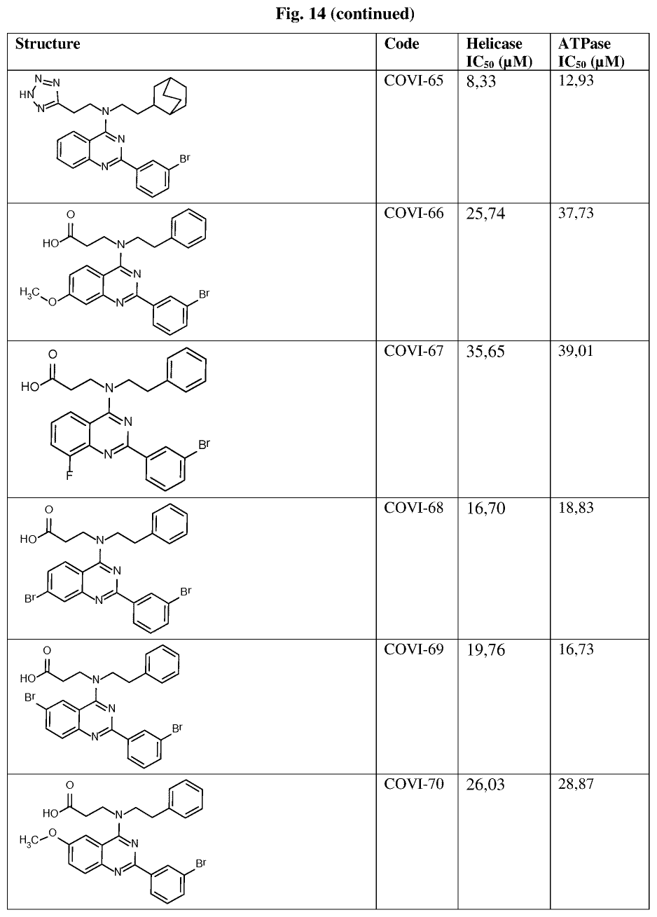

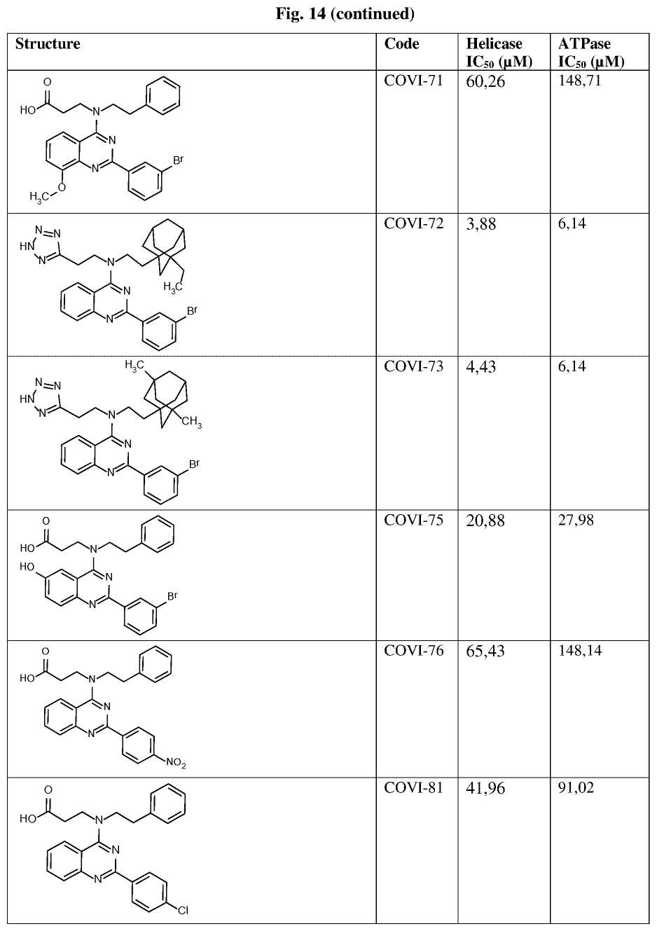

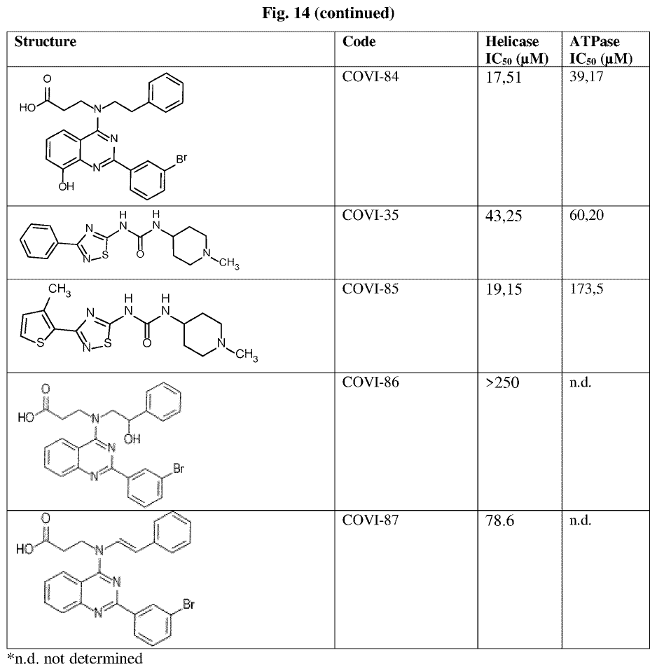

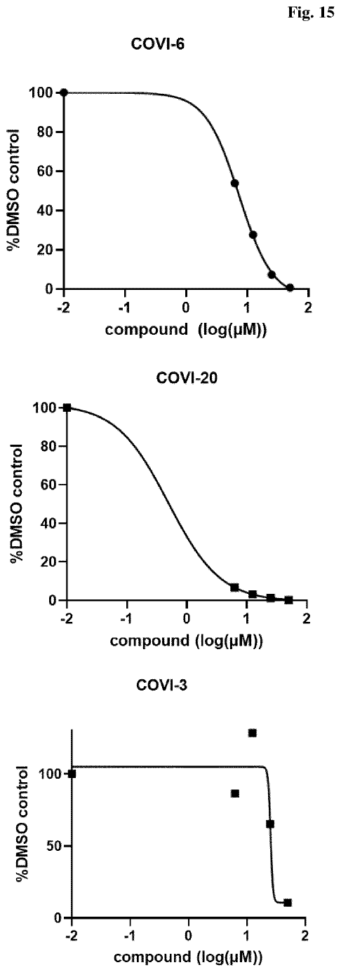

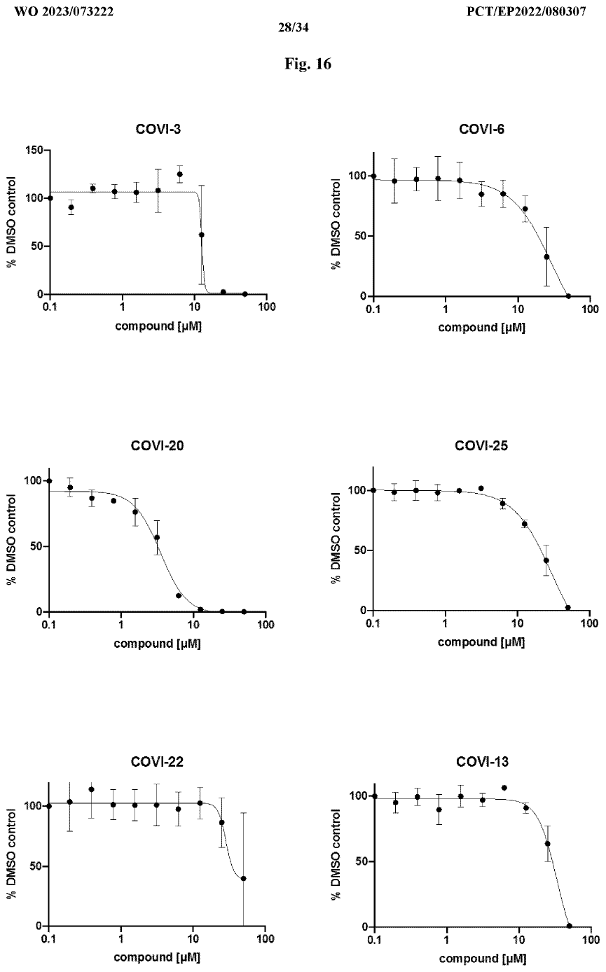

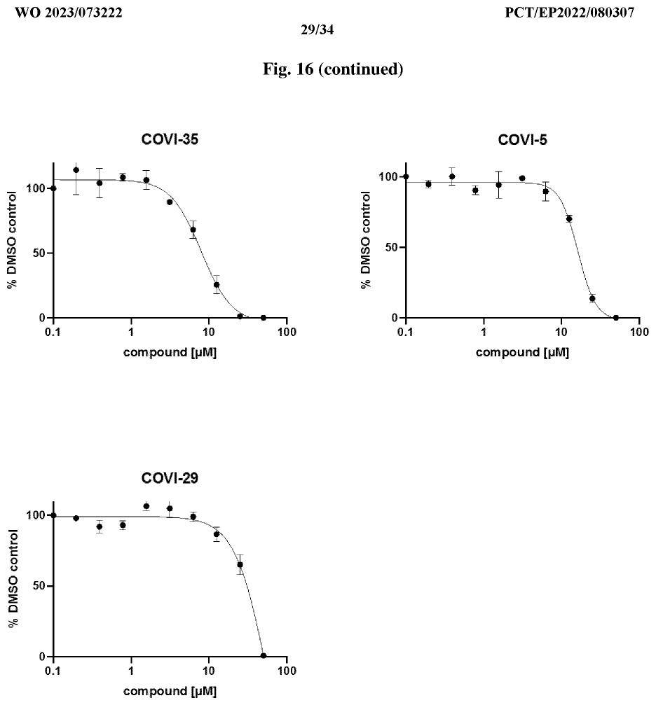

In addition to salt forms, the present invention provides compounds which are in a prodrug form. Prodrugs of the compounds described herein are those compounds that readily undergo chemical changes under physiological conditions to provide a compound of formula (I) to (IV), and especially a compound shown in FIG. 14. A prodrug is an active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. For a general discussion of prodrugs involving esters, see Svensson L. A. and Tunek A. (1988) Drug Metabolism Reviews 19(2): 165-194 and Bundgaard H. “Design of Prodrugs”, Elsevier Science Ltd. (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bundgaard H. et al. (1989) J. Med. Chem. 32(12): 2503-2507). Also, drugs containing an acidic NH group, such as imidazole, imide, indole and the like, have been masked with N-acyloxymethyl groups (Bundgaard H. “Design of Prodrugs”, Elsevier Science Ltd. (1985)). Hydroxy groups have been masked as esters and ethers. EP 0 039 051 A2 discloses Mannich-base hydroxamic acid prodrugs, their preparation and use.

Without wishing to be bound by a particular theory, the inventors believe that the active compound COVI-3 can be metabolized in vivo to COVI-86. COVI-86, in turn, can be further metabolized in vivo to the active compound COVI-87. Thus, COVI-86 can be viewed as a prodrug of the active compound COVI-87.

The compounds of the present invention may also contain unnatural proportions of atomic isotopes at one or more of the atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the present invention, whether radioactive or not, are intended to be encompassed within the scope of the present invention.

As used herein, “para position” when referring to the substituent of an aryl means that the substituent occupies the position opposite to the position at which the aryl is linked to the backbone of the compound.

As used herein, a “patient” means any mammal or bird that may benefit from a treatment with the compounds described herein. Preferably, a “patient” is selected from the group consisting of laboratory animals, domestic animals, or primates including chimpanzees and human beings. It is particularly preferred that the “patient” is a human being.

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease or disorder means preventing that a disorder occurs in a subject for a certain amount of time. For example, if a compound described herein is administered to a subject with the aim of preventing a disease or disorder, said disease or disorder is prevented from occurring at least on the day of administration and preferably also on one or more days (e.g. on 1 to 30 days; or on 2 to 28 days; or on 3 to 21 days; or on 4 to 14 days; or on 5 to 10 days) following the day of administration.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

An “effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

### Embodiments of the Invention

In the following paragraphs, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

The present inventors have identified and characterized within Nsp13 a pocket that appears to be involved in allosteric regulation of the ATPase activity of Nsp13. Compounds that specifically bind to this pocket are capable of inhibiting the ATPase activity of Nsp13. Compounds that bind to the ATPase site of Nsp13 and block the ATPase activity have to compete with ATP for binding to the ATPase site. Since the cellular ATP concentration is in the range of 1 to 10 mM depending on the cellular compartment, very high binding affinities in the low nanomolar range are required to successfully prevent ATP from binding to the ATPase site of Nsp13. The compounds reported by Mirza and Froeyen (supra) and those reported by Ugurel et al. (supra) Gurung (supra) are reported to bind in the ATP binding site. Specifically, Mirza and Froeyen report binding to residues 288, 289, 374, 375, 567, 178, 310, 312, 314, 378, and 539 while Gurung reports binding to amino acids 178, 284, 285, 286, 287, 288, 289, 290, 310, 312, 313, 316, 317, 320, 374, 375, 378, 404, 442, 443, 534, 535, 537, 538, 539, 540, 567, and 553. The binding site of the presently reported compounds and of ATP is separated from the allosteric pocket by two loops, from E144 to Y150 and from D171 to P178. These amino acids are thus capable of interactions both in the binding site of ATP as well as in the allosteric pocket, and indeed D374, E375, and M378 are reported as interacting with a number of the ATP competitive compounds reported by Mirza and Froeyen as well as Gurung and the allosteric inhibitors reported by the present inventors. Allosteric inhibitors of Nsp13 do not have this limitation since they do not have to prevent ATP from binding but inhibit Nsp13's ATPase activity through a different mechanism. The present inventors have identified compounds that are capable of specifically binding to the allosteric pocket and determined the spatial and electronic requirements of compounds that fit into this pocket. Thus, by defining the “lock” the inventors were able to define the “keys”, i.e. compounds, fitting into this lock, i.e. the allosteric binding pocket, and that are capable of forming non-covalent bonds or other stabilizing interactions to allow them to specifically bind in the pocket. Using this rational design approach the present inventors identified compounds that were capable of inhibiting Nsp13 movement on chromatin.

Structure-based computer modeling of ligand-protein interactions is now a core component of modern drug discovery (Charifson and Kuntz, 1997). Computational methods have played a key role in the drug discovery process for a growing number of marketed drugs, including HIV protease inhibitors (Charifson and Kuntz, 1997; Greer et al., 1994; Jorgensen, 2004) and zanamivir (an antiviral neuraminidase inhibitor) (von Itzstein et al., 1993), and in the development of new drug candidates, such as HIV integrase inhibitors (Hazuda et al., 2004; Schames et al., 2004), hepatitis C protease inhibitors (Liverton et al., 2008; Thomson and Perni, 2006), and beta-secretase inhibitors (BACE-1) (Stauffer et al., 2007). There are three major classes of physical computer methods available in the field (listed from fastest to slowest, and least physical to most physical): (1) very fast molecular docking methods, including DOCK, Glide, AutoDock, FlexX, ICN, PMF, and GOLD, Molecular dynamics based free energy methods (MD), such as MM/GBSA or MM/PBSA, in which solvent, protein, and ligand are subject to forces exerted by each other and thermal fluctuations and move in iterative steps as a response to these forces, and (4) absolute binding free energy (ABFE) methods, including alchemical simulations, which are the most expensive computationally, but which include the physics in the most rigorous way that is currently practical. ABFE methods start from an unbound ligand and potentially the unbound structure of the protein to attempt to predict the structures, affinities, and thermal properties of the complexes of interest. These strategies known in the art and in particular the approach described in the experimental section can be used to identify compounds that are allosteric inhibitors of ALC1 by binding to the allosteric binding pocket within ALC1 first identified by the present inventors.

Thus, in a first aspect the present invention relates to an inhibitor of the helicase activity of a Nsp13 helicase of SARS-CoV-2 (Nsp13) or of a viral homologue thereof, wherein the inhibitor specifically binds to an allosteric binding pocket within the N-terminal lobe of the ATPase domain of Nsp13 or of a viral homologue thereof.

The term “an inhibitor of the helicase activity” as used in the context of the inhibitors of the present invention refers to the inhibition of the activity of a helicase of a virus of the class of positive-strand RNA viruses, preferably of the class Pisoniviricetes, more preferably of the order of Nidovirales and more preferably of the family of Coronaviridae. To assess the ability of an individual compound to inhibit helicase activity it is preferred that the helicase activity is determined in a helicase unwinding assay using SARSCoV2 helicase Nsp13 with an amino acid sequence according to SEQ ID NO: 1 as described herein. Such an assay preferably uses 0.15 nM Nsp13 and 100 nM of dsDNA that is labeled with a pair of FRET labels to detect the unwinding of the dsDNA. The unwinding reaction can be initiated by the addition of 200 μM ATP and 1 μM of unlabeled single-stranded DNA. It is preferred that inhibitors of the invention inhibit the helicase activity with an IC₅₀ of 100 M or less, more preferably with an IC₅₀ of 50 M or less and more preferably with an IC₅₀ of 20 M or less and most preferably with an IC₅₀ of 10 M or less.

The term “viral homologue” of SARS-CoV-2 Nsp13 with an amino acid sequence according to SEQ ID NO: 1 refers to viral proteins that are functional homologs, i.e. that exhibit helicase activity in a chromatin remodeling assay as used in the examples of the present invention (see Example 3). Preferably, the term refers to a protein that is a functional and structural homologue of SARS-CoV-2 Nsp13. The structural homology is preferably within the amino acids of the viral homologue that form the allosteric pocket of the helicase. The present inventors have used molecular dynamics (MD) simulation on the first lobe of the SARS-CoV-1 Nsp13 helicase (residues 236 to 440 from the published SARS-CoV-1 Nsp13 PDB: 6JYT, see Example 1) to identify a yet unidentified allosteric binding pocket within Nsp13. The allosteric binding pocket to which the helicase inhibitors of the present invention bind is formed by amino acids 236 to 440 of Nsp13 according to SEQ ID NO: 1. However, not every amino acid within the amino acid stretch spanning amino acid residues 236 to 440 of SEQ ID NO: 1 is forming the surface of the allosteric pocket of Nsp13 available for binding to the inhibitors of the invention. This is due to the fact that some amino acids are buried in the pocket and are poorly accessible and others are not even part of the pocket but are located within Nsp13 or on the outside surface of Nsp13. The surface area of this allosteric binding pocket of Nsp13 is formed by the following amino acids (with reference to SEQ ID NO: 1): Y277, T279, L280, Q281, G282, T307, C309, F373, D374, E375, I376, S377, M378, A379, L384, N388, Y396, Y398, I399, G400, D401, P406, P408, N423, V425, C426, R427, and M429. Of these residues amino acids Y277, L384, N388, Y396, Y398, and V425 form the entry channel into the pocket through which the inhibitors of the invention may enter the binding pocket, and amino acids T307, C309, F373, D374, E375, S377, M378, A379, and N423 form the left channel and T279, L280, Q281, G282, I376, I399, G400, D401, P406, P408, C426, R427, and M429 form the right channel. Thus, preferred viral homologues comprise a binding pocket of similar structure, which can be assessed by using MD as described below. Preferred structural homologues are those that comprise identical or conservatively or semi-conservatively substituted amino acids at at least 50% of the amino acid positions corresponding to Y277, T279, L280, Q281, G282, T307, C309, F373, D374, E375, I376, S377, M378, A379, L384, N388, Y396, Y398, I399, G400, D401, P406, P408, N423, V425, C426, R427, and M429 of Nsp13 with an amino acid sequence according to SEQ ID NO: 1. Preferred structural homologues comprise identical or conservatively substituted amino acids at at least 60%, 70%, 80% of the amino acid positions corresponding to Y277, T279, L280, Q281, G282, T307, C309, F373, D374, E375, I376, S377, M378, A379, L384, N388, Y396, Y398, I399, G400, D401, P406, P408, N423, V425, C426, R427, and M429 of Nsp13 with an amino acid sequence according to SEQ ID NO: 1. Particularly preferred structural viral helicase homologues share at least 60%, at least 70%, more preferably at least 80%, more preferably at least 90% with AA 277 to M429 of Nsp13 with an amino acid sequence according to SEQ ID NO: 1. Exemplary preferred viral homologues of SARS-CoV-2 Nsp13 are shown in FIG. 3 and are depicted in the appended sequence listing as SEQ ID NOs: 2 to 7.

The term “corresponding position” is used in the context of the present invention to refer to an amino acid position within the amino acid sequence of a given protein (e.g. a helicase homologue of Nsp13 according to SEQ ID NO: 1), which is aligned with a reference protein, in particular with Nsp13 of SARSCoV2 according to SEQ ID NO: 1, that aligns with an amino acid in the reference protein. Alignments of two or more amino acids sequences can be carried out using a number of publicly available software tools including Clustal Omega (https://www.ebi.ac.uk/Tools/msa/clustalo/) or PBLAST in each case using standard alignment parameters. An example of such an alignment using Nsp13 of SARSCoV2 according to SEQ ID NO: 1 and helicases of six related RNA viruses are shown in FIG. 3. The skilled person can readily determine on the basis of such an alignment an amino acid that corresponds to one of the amino acids specifically indicated above and below with reference to Nsp13 of SARSCoV2 according to SEQ ID NO: 1. For example, with Y277 of SARSCoV2 aligns with 1278 of SARSCoV (SEQ ID NO: 2) referred to as “229E” in FIG. 3, with 1278 of Nsp13 helicase of HCoV-NL63 (SEQ ID NO: 3) referred to as “NL-63” in FIG. 3, with Y276 of Nsp13 helicase of HCoV OC43 (SEQ ID NO: 4) referred to as “OC43” in FIG. 3, with Y276 of Nsp13 helicase of HCoV HKU1 (SEQ ID NO: 5) referred to as “HKU1” in figure, with Y277 of Nsp13 helicase of MERS CoV (SEQ ID NO: 6), and with Y277 of Nsp13 helicase of SARS CoV (SEQ ID NO: 7).

Accordingly, in one embodiment of the first aspect,
(i) the N-terminal lobe of the ATPase domain consists of amino acid residues 236 to 440 of SEQ ID NO: 1 or the corresponding amino acid residues of the viral homologue;
and/or
(ii) the allosteric binding pocket comprises or consists of Y277, T279, L280, Q281, G282, T307, C309, F373, D374, E375, I376, S377, M378, A379, L384, N388, Y396, Y398, I399, G400, D401, P406, P408, N423, V425, T426, R427, and M429 of Nsp13 of SARSCoV2 according to SEQ ID NO: 1 or of the amino acids at the corresponding positions in a viral homologue of Nsp13. These are the amino acids that can form non-covalent bonds with the inhibitor of the present invention because their side chains and/or back bones form the surface of the binding pocket.

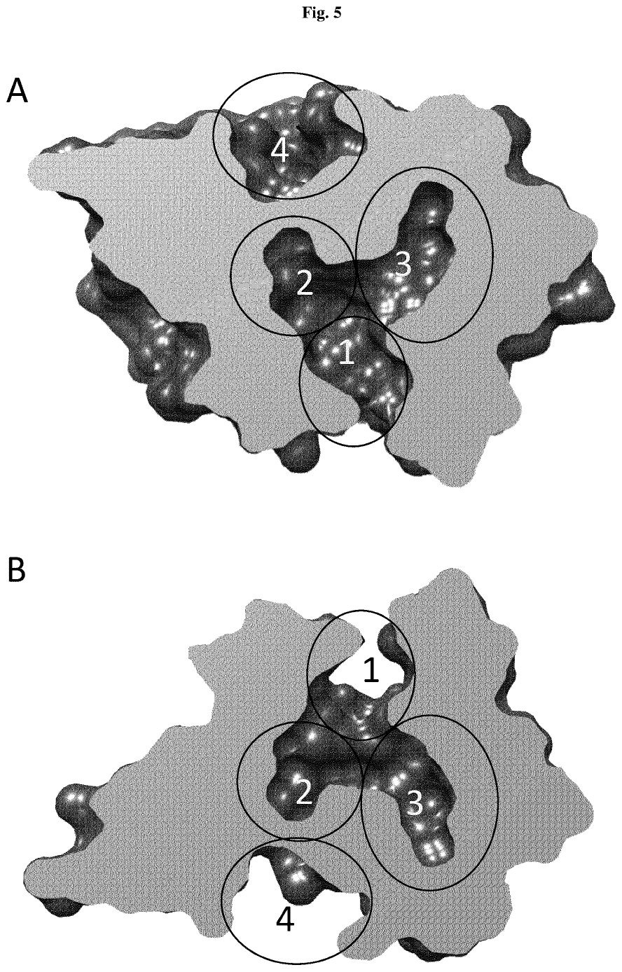

In a further embodiment of the first aspect, the allosteric binding pocket is tripartite and comprises an entry channel (1), a left channel (2) and a right channel (3) and is located on the backside of the active site of Nsp13 or of a viral homologue thereof. It can be seen in FIG. 5 to 7, and 10. An allosteric inhibitor of the invention specifically binds, preferably non-covalently, to amino acids within one or more of the entry channel (1), the left channel (2), and the right channel (3) and, thereby inhibits allosteric activation of the helicase.

In a further embodiment of the first aspect, the inhibitor specifically binds to one or more (for example to 1, 2, 3, 4, 5, 6 or all 7) of the following seven amino acid residues in the allosteric binding pocket of Nsp13 of SARSCoV2 according to SEQ ID NO: 1 or of the amino acids at the corresponding positions in a viral homologue of Nsp13: F373, I376, L384, Y398, I399, V425, and M429.

In a further embodiment of the first aspect, the inhibitor specifically binds to at least one of the following seven sets of amino acids within the allosteric binding pocket:
(i) in the entry channel the backbone of at least one amino acid residues selected from N381, L384, S385, V387, N388, and V425 of Nsp13 of SARSCoV2 according to SEQ ID NO: 1 or of the amino acids at the corresponding positions in a viral homologue of Nsp13 and/or one or more sidechains of amino acid residues selected from P238, T239, Y277, N381, L384, S385, V387, N388, Y396, Y398, N 423, and V425 of Nsp13 of SARSCoV2 according to SEQ ID NO: 1 or of the amino acids at the corresponding positions in a viral homologue of Nsp13;
(ii) in the right channel the backbone of at least one amino acid residues selected from L280, T279, Q281, G282, T307, C309, F373, Y398, I399, G400, D401, P406, P408, V425, C426, and R427 of Nsp13 of SARSCoV2 according to SEQ ID NO: 1 or of the amino acids at the corresponding positions in a viral homologue of Nsp13 and/or one or more side chains of amino acid residues selected from L280, Q281, F373, I376, I399, G400, P406, P408, N423, C426, and M429 of Nsp13 of SARSCoV2 according to SEQ ID NO: 1 or of the amino acids at the corresponding positions in a viral homologue of Nsp13; (iii) in the left channel the backbone of amino acid residues selected from T279, E375, I376, S377, M378, A379, P408, and M429 of Nsp13 of SARSCoV2 according to SEQ ID NO: 1 or of the amino acids at the corresponding positions in a viral homologue of Nsp13 and/or one or more sidechains of amino acid residues selected from T307, C309, F373, D374, I376, M378, A379, L384, and N423 of Nsp13 of SARSCoV2 according to SEQ ID NO: 1 or of the amino acids at the corresponding positions in a viral homologue of Nsp13;
(iv) in the right and entry channel the backbone of at least one amino acid residue selected from L280, T279, Q281, G282, T307, C309, F373, N381, L384, S385, V387, N388, Y398, I399, G400, D401, P406, P408, V425, C426, and R427 of Nsp13 of SARSCoV2 according to SEQ ID NO: 1 or of the amino acids at the corresponding positions in a viral homologue of Nsp13 and/or one or more sidechains of amino acid residues selected from P238, T239, Y277, L280, Q281, F373, I376, N381, L384, S385, V387, N388, Y396, Y398, I399, G400, P406, P408, N423, V425, C426, and M429 of Nsp13 of SARSCoV2 according to SEQ ID NO: 1 or of the amino acids at the corresponding positions in a viral homologue of Nsp13; (v) in the left and entry channel the backbone of amino acid residues selected from T279, E375, 1376, S377, M378, A379, N381, L384, S385, V387, N388, P408, V425, and M429 of Nsp13 of SARSCoV2 according to SEQ ID NO: 1 or of the amino acids at the corresponding positions in a viral homologue of Nsp13 and/or one or more sidechains of amino acid residues selected from P238, T239, Y277, T307, C309, F373, D374, I376, M378, A379, N381, L384, S385, V387, N388, Y396, Y398, N423, and V425 of Nsp13 of SARSCoV2 according to SEQ ID NO: 1 or of the amino acids at the corresponding positions in a viral homologue of Nsp13;
(vi) in the left and right channel the backbone of amino acid residues selected from T279, L280, Q281, G282, T307, C309, F373, E375, I376, S377, M378, A379, Y398, I399, G400, D401, P406, P408, V425, C426, R427, M429 of Nsp13 of SARSCoV2 according to SEQ ID NO: 1 or of the amino acids at the corresponding positions in a viral homologue of Nsp13 and/or one or more sidechains of amino acid residues selected from L280, Q281, T307, C309, F373, D374, I376, M378, A379, L384, I399, G400, P406, P408, N423, C426, and M429 of Nsp13 of SARSCoV2 according to SEQ ID NO: 1 or of the amino acids at the corresponding positions in a viral homologue of Nsp13; and/or
(vii) in the left, right and entry channel the backbone of amino acid residues selected from T279, L280, Q281, G282, T307, C309, F373, E375, I376, S377, M378, A379, N381, L384, S385, V387, N388, Y398, I399, G400, D401, P406, P408, V425, C426, R427, and M429 of Nsp13 of SARSCoV2 according to SEQ ID NO: 1 or of the amino acids at the corresponding positions in a viral homologue of Nsp13 and sidechains of residues P238, T239, Y277, L280, Q281, T307, C309, F373, D374, I376, M378, A379, N381, L384, S385, V387, N388, Y396, Y398, I399, G400, P406, P408, N423, V425, C426, and M429 of Nsp13 of SARSCoV2 according to SEQ ID NO: 1 or of the amino acids at the corresponding positions in a viral homologue of Nsp13.

In a further embodiment of the first aspect, the inhibitor specifically binds to:
(a) the amino acids indicated above in (i) and further to one more amino acids selected from the group consisting of Y277 of Nsp13 of SARSCoV2 according to SEQ ID NO: 1 or of the amino acids at the corresponding positions in a viral homologue of Nsp13, wherein the inhibitor binds to the aromatic ring of Y277 via face-to-face or edge-to-face pi-pi interaction with aromatic carbo- or heterocyclic substituents, or wherein the inhibitor binds to Y277 by cation-pi, polar-pi, or halogen-pi interactions with polar, charged, or carbo-halogen substituents; or wherein the terminal oxygen of Y277 interacts with a hydrogen bond donating group; N388 of Nsp13 of SARSCoV2 according to SEQ ID NO: 1 or of the amino acids at the corresponding positions in a viral homologue of Nsp13, wherein the inhibitor binds to a side chain on N388 via a hydrogen bond donating or accepting group; Y396 of Nsp13 of SARSCoV2 according to SEQ ID NO: 1 or of the amino acids at the corresponding positions in a viral homologue of Nsp13, wherein the inhibitor binds to the aromatic ring of Y396 via face-to-face or edge-to-face pi-pi interaction with aromatic carbo- or heterocyclic substituents, or wherein Y396 forms cation-pi, polar-pi, or halogen-pi interactions with polar, charged, or carbo-halogen substituents; and Y398 of Nsp13 of SARSCoV2 according to SEQ ID NO: 1 or of the amino acids at the corresponding positions in a viral homologue of Nsp13, wherein the inhibitors binds to the aromatic ring of Y398 via face-to-face or edge-to-face pi-pi interaction with aromatic carbo- or heterocyclic substituents or forms cation-pi, polar-pi, or halogen-pi interactions with polar, charged, or carbo-halogen substituents;
(b) the amino acids indicated above in (ii) and further to one more amino acids selected from the group consisting of L280 of Nsp13 of SARSCoV2 according to SEQ ID NO: 1 or of the amino acids at the corresponding positions in a viral homologue of Nsp13, wherein the inhibitor binds to the backbone N of L280 of Nsp13 of SARSCoV2 according to SEQ ID NO: 1 or of the amino acids at the corresponding positions in a viral homologue of Nsp13 with a hydrogen bond acceptor group; Y398 of Nsp13 of SARSCoV2 according to SEQ ID NO: 1 or of the amino acids at the corresponding positions in a viral homologue of Nsp13, wherein the inhibitor binds to the aromatic ring of Y398 via face-to-face or edge-to-face pi-pi interaction with aromatic carbo- or heterocyclic substituents or via cation-pi, polar-pi, or halogen-pi interactions with polar, charged, or carbo-halogen substituents; 1399 of Nsp13 of SARSCoV2 according to SEQ ID NO: 1 or of the amino acids at the corresponding positions in a viral homologue of Nsp13, wherein the inhibitor binds to the backbone carbonyl oxygen of 1399 by carbo halogen or hydrogen bond donating groups or to the backbone N of 1399 interacting with a hydrogen bond acceptor; G400 of Nsp13 of SARSCoV2 according to SEQ ID NO: 1 or of the amino acids at the corresponding positions in a viral homologue of Nsp13, wherein the inhibitor binds to the backbone N of G400 with a hydrogen bond acceptor group or to the backbone carbonyl oxygen of G400 with carbo halogen or hydrogen bond donating groups; D401 of Nsp13 of SARSCoV2 according to SEQ ID NO: 1 or of the amino acids at the corresponding positions in a viral homologue of Nsp13, wherein the inhibitor binds to the backbone carbonyl oxygen of D401 with carbo halogen or hydrogen bond donating groups; P406, wherein the inhibitor binds to the backbone carbonyl oxygen of P406 with carbo halogen or hydrogen bond donating groups; and C426 of Nsp13 of SARSCoV2 according to SEQ ID NO: 1 or of the amino acids at the corresponding positions in a viral homologue of Nsp13, wherein the inhibitor binds to the S of C426 with a carbo-halogen group or the S of C426 forms a covalent bond to a “warhead” of the inhibitor;
(c) the amino acids indicated above in (iii) and further to one more amino acids selected from the group consisting of T307 of Nsp13 of SARSCoV2 according to SEQ ID NO: 1 or of the amino acids at the corresponding positions in a viral homologue of Nsp13, wherein the inhibitor binds to the sidechain of T307 with a hydrogen bond donating or accepting group or wherein the inhibitor binds to the backbone carbonyl oxygen of T307 with carbo halogens or hydrogen bond donating groups; F373 of Nsp13 of SARSCoV2 according to SEQ ID NO: 1 or of the amino acids at the corresponding positions in a viral homologue of Nsp13, wherein the inhibitor binds to the aromatic ring of F373 via face-to-face or edge-to-face pi-pi interaction with aromatic carbo- or heterocyclic substituents or via cation-pi, polar-pi, or halogen-pi interactions with polar, charged, or carbo-halogen substituents; C309 of Nsp13 of SARSCoV2 according to SEQ ID NO: 1 or of the amino acids at the corresponding positions in a viral homologue of Nsp13, wherein the inhibitor binds to the S of C309 with carbo-halogens or the S of C309 forms a covalent bond to a “warhead” of the inhibitor; D374 of Nsp13 of SARSCoV2 according to SEQ ID NO: 1 or of the amino acids at the corresponding positions in a viral homologue of Nsp13, wherein the inhibitor binds to the sidechain of D374 with a hydrogen bond donating or accepting group; E375 of Nsp13 of SARSCoV2 according to SEQ ID NO: 1 or of the amino acids at the corresponding positions in a viral homologue of Nsp13, wherein the inhibitor binds to the backbone carbonyl oxygen of E375 with carbo halogens or hydrogen bond donating groups; 1376 of Nsp13 of SARSCoV2 according to SEQ ID NO: 1 or of the amino acids at the corresponding positions in a viral homologue of Nsp13, wherein the inhibitor binds to the backbone carbonyl oxygen of 1376 with carbo halogens or hydrogen bond donating groups; M378 of Nsp13 of SARSCoV2 according to SEQ ID NO: 1 or of the amino acids at the corresponding positions in a viral homologue of Nsp13, wherein the inhibitor binds to the backbone N of M378 with a hydrogen bond acceptor group; and A379 of Nsp13 of SARSCoV2 according to SEQ ID NO: 1 or of the amino acids at the corresponding positions in a viral homologue of Nsp13, wherein the inhibitor binds to the backbone carbonyl oxygen of A379 with carbo halogens or hydrogen bond donating groups or to the backbone N of A379 with a hydrogen bond acceptor group;
(d) the amino acids indicated above in (iv) and further to one more amino acids selected from the group consisting of Y277 of Nsp13 of SARSCoV2 according to SEQ ID NO: 1 or of the amino acids at the corresponding positions in a viral homologue of Nsp13, wherein the inhibitor binds to the aromatic ring of Y277 via face-to-face or edge-to-face pi-pi interaction with aromatic carbo- or heterocyclic substituents or via cation-pi, polar-pi, or halogen-pi interactions with polar, charged, or carbo-halogen substituents or wherein the inhibitor binds to the terminal oxygen of Y277 with a hydrogen bond donating group; L280 of Nsp13 of SARSCoV2 according to SEQ ID NO: 1 or of the amino acids at the corresponding positions in a viral homologue of Nsp13, wherein the inhibitor binds to the backbone N of L280 with a hydrogen bond acceptor group; N388 of Nsp13 of SARSCoV2 according to SEQ ID NO: 1 or of the amino acids at the corresponding positions in a viral homologue of Nsp13, wherein the inhibitor binds to the side chain of N388 with a hydrogen bond donating or accepting group; Y396 of Nsp13 of SARSCoV2 according to SEQ ID NO: 1 or of the amino acids at the corresponding positions in a viral homologue of Nsp13, wherein the inhibitor binds to the aromatic ring of Y396 via face-to-face or edge-to-face pi-pi interaction with aromatic carbo- or heterocyclic substituents or via cation-pi, polar-pi, or halogen-pi interactions with polar, charged, or carbo-halogen substituents; Y398 of Nsp13 of SARSCoV2 according to SEQ ID NO: 1 or of the amino acids at the corresponding positions in a viral homologue of Nsp13, wherein the inhibitor binds to the aromatic ring of Y398 via face-to-face or edge-to-face pi-pi interaction with aromatic carbo- or heterocyclic substituents or via cation-pi, polar-pi, or halogen-pi interactions with polar, charged, or carbo-halogen substituents; 1399 of Nsp13 of SARSCoV2 according to SEQ ID NO: 1 or of the amino acids at the corresponding positions in a viral homologue of Nsp13, wherein the inhibitor binds to the backbone carbonyl oxygen of 1399 with carbo halogen or hydrogen bond donating groups or to the backbone N of 1399 with a hydrogen bond acceptor group; G400 of Nsp13 of SARSCoV2 according to SEQ ID NO: 1 or of the amino acids at the corresponding positions in a viral homologue of Nsp13, wherein the inhibitor binds to the backbone N of G400 with a hydrogen bond acceptor group or to the backbone carbonyl oxygen of G400 with carbo halogen or hydrogen bond donating groups; D401 of Nsp13 of SARSCoV2 according to SEQ ID NO: 1 or of the amino acids at the corresponding positions in a viral homologue of Nsp13, wherein the inhibitor binds to the backbone carbonyl oxygen of D401 with carbo halogen or hydrogen bond donating groups; P406 of Nsp13 of SARSCoV2 according to SEQ ID NO: 1 or of the amino acids at the corresponding positions in a viral homologue of Nsp13, wherein the inhibitor binds to the backbone carbonyl oxygen of P406 with carbo halogen or hydrogen bond donating groups; and C426 of Nsp13 of SARSCoV2 according to SEQ ID NO: 1 or of the amino acids at the corresponding positions in a viral homologue of Nsp13, wherein the inhibitor binds to the S of C426 with a carbo-halogen or the S of 426 forms a covalent bond to a “warhead” of the inhibitor;
(e) the amino acids indicated above in (v) and further to one more amino acids selected from the group consisting of: Y277 of Nsp13 of SARSCoV2 according to SEQ ID NO: 1 or of the amino acids at the corresponding positions in a viral homologue of Nsp13, wherein the inhibitor binds to the aromatic ring of Y277 via face-to-face or edge-to-face pi-pi interaction with aromatic carbo- or heterocyclic substituents or via cation-pi, polar-pi, or halogen-pi interactions with polar, charged, or carbo-halogen substituents or wherein the inhibitor binds to the terminal oxygen of Y277 with a hydrogen bond donating group; N388 of Nsp13 of SARSCoV2 according to SEQ ID NO: 1 or of the amino acids at the corresponding positions in a viral homologue of Nsp13, wherein the inhibitor binds to the side chain of N388 with a hydrogen bond donating or accepting group; T307 of Nsp13 of SARSCoV2 according to SEQ ID NO: 1 or of the amino acids at the corresponding positions in a viral homologue of Nsp13, wherein the inhibitor binds to the sidechain of T307 with a hydrogen bond donating or accepting group or to the backbone carbonyl oxygen of T307 with carbo halogens or hydrogen bond donating groups; F373 of Nsp13 of SARSCoV2 according to SEQ ID NO: 1 or of the amino acids at the corresponding positions in a viral homologue of Nsp13, wherein the inhibitor binds to the aromatic ring of F373 via face-to-face or edge-to-face pi-pi interaction with aromatic carbo- or heterocyclic substituents or via cation-pi, polar-pi, or halogen-pi interactions with polar, charged, or carbo-halogen substituents; C309 of Nsp13 of SARSCoV2 according to SEQ ID NO: 1 or of the amino acids at the corresponding positions in a viral homologue of Nsp13, wherein the inhibitor binds to the S of C309 with carbo-halogens or the S of C309 forms a covalent bond to a “warhead” of the inhibitor; D374 of Nsp13 of SARSCoV2 according to SEQ ID NO: 1 or of the amino acids at the corresponding positions in a viral homologue of Nsp13, wherein the inhibitor binds to the sidechain of D374 with a hydrogen bond donating or accepting group; E375 of Nsp13 of SARSCoV2 according to SEQ ID NO: 1 or of the amino acids at the corresponding positions in a viral homologue of Nsp13, wherein the inhibitor binds to the backbone carbonyl oxygen of E375 with carbo halogens or hydrogen bond donating groups; 1376, wherein the inhibitor binds to the backbone carbonyl oxygen of 1376 with carbo halogens or hydrogen bond donating groups; M378 of Nsp13 of SARSCoV2 according to SEQ ID NO: 1 or of the amino acids at the corresponding positions in a viral homologue of Nsp13, wherein the inhibitor binds to the backbone N of M378 with a hydrogen bond acceptor group; A379 of Nsp13 of SARSCoV2 according to SEQ ID NO: 1 or of the amino acids at the corresponding positions in a viral homologue of Nsp13, wherein the inhibitor binds to the backbone carbonyl oxygen of A379 with carbo halogens or hydrogen bond donating groups or to the backbone N of A379 with a hydrogen bond acceptor group; Y396, of Nsp13 of SARSCoV2 according to SEQ ID NO: 1 or of the amino acids at the corresponding positions in a viral homologue of Nsp13 wherein the inhibitor binds to the aromatic ring of Y396 via face-to-face or edge-to-face pi-pi interaction with aromatic carbo- or heterocyclic substituents or via cation-pi, polar-pi, or halogen-pi interactions with polar, charged, or carbo-halogen substituents; and Y398 of Nsp13 of SARSCoV2 according to SEQ ID NO: 1 or of the amino acids at the corresponding positions in a viral homologue of Nsp13, wherein the inhibitor binds to the aromatic ring of Y398 via face-to-face or edge-to-face pi-pi interaction with aromatic carbo- or heterocyclic substituents or via cation-pi, polar-pi, or halogen-pi interactions with polar, charged, or carbo-halogen substituents;
(f) the amino acids indicated above in (vi) and further to one more amino acids selected from the group consisting of L280 of Nsp13 of SARSCoV2 according to SEQ ID NO: 1 or of the amino acids at the corresponding positions in a viral homologue of Nsp13, wherein the inhibitor binds to the backbone N of L280 with a hydrogen bond acceptor group; T307 of Nsp13 of SARSCoV2 according to SEQ ID NO: 1 or of the amino acids at the corresponding positions in a viral homologue of Nsp13, wherein the inhibitor binds to the sidechain of T307 with a hydrogen bond donating or accepting group or to the backbone carbonyl oxygen of T307 with carbo halogens or hydrogen bond donating groups; F373 of Nsp13 of SARSCoV2 according to SEQ ID NO: 1 or of the amino acids at the corresponding positions in a viral homologue of Nsp13, wherein the inhibitor binds to the aromatic ring of F373 via face-to-face or edge-to-face pi-pi interaction with aromatic carbo- or heterocyclic substituents or via cation-pi, polar-pi, or halogen-pi interactions with polar, charged, or carbo-halogen substituents; C309, wherein the inhibitor binds to the S of C309 with carbo-halogens or the S of C309 forms a covalent bond to a “warhead” of the inhibitor; D374 of Nsp13 of SARSCoV2 according to SEQ ID NO: 1 or of the amino acids at the corresponding positions in a viral homologue of Nsp13, wherein the inhibitor binds to the sidechain of D374 with a hydrogen bond donating or accepting group; E375, wherein the inhibitor binds to the backbone carbonyl oxygen of E375 with carbo halogens or hydrogen bond donating groups; 1376 of Nsp13 of SARSCoV2 according to SEQ ID NO: 1 or of the amino acids at the corresponding positions in a viral homologue of Nsp13, wherein the inhibitor binds to the backbone carbonyl oxygen of 1376 with carbo halogens or hydrogen bond donating groups; M378 of Nsp13 of SARSCoV2 according to SEQ ID NO: 1 or of the amino acids at the corresponding positions in a viral homologue of Nsp13, wherein the inhibitor binds to the backbone N of M378 with a hydrogen bond acceptor group; A379 of Nsp13 of SARSCoV2 according to SEQ ID NO: 1 or of the amino acids at the corresponding positions in a viral homologue of Nsp13, wherein the inhibitor binds to the backbone carbonyl oxygen of A379 with carbo halogens or hydrogen bond donating groups or to the backbone N of A379 with a hydrogen bond acceptor group; Y398 of Nsp13 of SARSCoV2 according to SEQ ID NO: 1 or of the amino acids at the corresponding positions in a viral homologue of Nsp13, wherein the inhibitor binds to the aromatic ring of Y398 via face-to-face or edge-to-face pi-pi interaction with aromatic carbo- or heterocyclic substituents or via cation-pi, polar-pi, or halogen-pi interactions with polar, charged, or carbo-halogen substituents; 1399 of Nsp13 of SARSCoV2 according to SEQ ID NO: 1 or of the amino acids at the corresponding positions in a viral homologue of Nsp13, wherein the inhibitor binds to the backbone carbonyl oxygen of 1399 with carbo halogen or hydrogen bond donating groups or to the backbone N of 1399 with a hydrogen bond acceptor group; G400 of Nsp13 of SARSCoV2 according to SEQ ID NO: 1 or of the amino acids at the corresponding positions in a viral homologue of Nsp13, wherein the inhibitor binds to the backbone N of G400 with a hydrogen bond acceptor group or to the backbone carbonyl oxygen of G400 with carbo halogen or hydrogen bond donating groups; D401 of Nsp13 of SARSCoV2 according to SEQ ID NO: 1 or of the amino acids at the corresponding positions in a viral homologue of Nsp13, wherein the inhibitor binds to the backbone carbonyl oxygen of D401 with carbo halogen or hydrogen bond donating groups; P406, wherein the inhibitor binds to the backbone carbonyl oxygen of P406 with carbo halogen or hydrogen bond donating groups; and C426 of Nsp13 of SARSCoV2 according to SEQ ID NO: 1 or of the amino acids at the corresponding positions in a viral homologue of Nsp13, wherein the inhibitor binds to the S of C426 with a carbo-halogen or the S of 426 forms a covalent bond to a “warhead” of the inhibitor; or
(g) the amino acids indicated above in (vii) and further to one more amino acids selected from the group consisting of Y277 of Nsp13 of SARSCoV2 according to SEQ ID NO: 1 or of the amino acids at the corresponding positions in a viral homologue of Nsp13, wherein the inhibitor binds to the aromatic ring of Y277 via face-to-face or edge-to-face pi-pi interaction with aromatic carbo- or heterocyclic substituents or via cation-pi, polar-pi, or halogen-pi interactions with polar, charged, or carbo-halogen substituents or wherein the inhibitor binds to the terminal oxygen of Y277 with a hydrogen bond donating group; N388 of Nsp13 of SARSCoV2 according to SEQ ID NO: 1 or of the amino acids at the corresponding positions in a viral homologue of Nsp13, wherein the inhibitor binds to the side chain of N388 with a hydrogen bond donating or accepting group; Y396 of Nsp13 of SARSCoV2 according to SEQ ID NO: 1 or of the amino acids at the corresponding positions in a viral homologue of Nsp13, wherein the inhibitor binds to the aromatic ring of Y396 via face-to-face or edge-to-face pi-pi interaction with aromatic carbo- or heterocyclic substituents or via cation-pi, polar-pi, or halogen-pi interactions with polar, charged, or carbo-halogen substituents; L280 of Nsp13 of SARSCoV2 according to SEQ ID NO: 1 or of the amino acids at the corresponding positions in a viral homologue of Nsp13, wherein the inhibitor binds to the backbone N of L280 with a hydrogen bond acceptor group; T307 of Nsp13 of SARSCoV2 according to SEQ ID NO: 1 or of the amino acids at the corresponding positions in a viral homologue of Nsp13, wherein the inhibitor binds to the sidechain of T307 with a hydrogen bond donating or accepting group or to the backbone carbonyl oxygen of T307 with carbo halogens or hydrogen bond donating groups; F373 of Nsp13 of SARSCoV2 according to SEQ ID NO: 1 or of the amino acids at the corresponding positions in a viral homologue of Nsp13, wherein the inhibitor binds to the aromatic ring of F373 via face-to-face or edge-to-face pi-pi interaction with aromatic carbo- or heterocyclic substituents or via cation-pi, polar-pi, or halogen-pi interactions with polar, charged, or carbo-halogen substituents; C309 of Nsp13 of SARSCoV2 according to SEQ ID NO: 1 or of the amino acids at the corresponding positions in a viral homologue of Nsp13, wherein the inhibitor binds to the S of C309 with carbo-halogens or the S of C309 forms a covalent bond to a “warhead” of the inhibitor; D374 of Nsp13 of SARSCoV2 according to SEQ ID NO: 1 or of the amino acids at the corresponding positions in a viral homologue of Nsp13, wherein the inhibitor binds to the sidechain of D374 with a hydrogen bond donating or accepting group; E375 of Nsp13 of SARSCoV2 according to SEQ ID NO: 1 or of the amino acids at the corresponding positions in a viral homologue of Nsp13, wherein the inhibitor binds to the backbone carbonyl oxygen of E375 with carbo halogens or hydrogen bond donating groups; 1376 of Nsp13 of SARSCoV2 according to SEQ ID NO: 1 or of the amino acids at the corresponding positions in a viral homologue of Nsp13, wherein the inhibitor binds to the backbone carbonyl oxygen of 1376 with carbo halogens or hydrogen bond donating groups; M378 of Nsp13 of SARSCoV2 according to SEQ ID NO: 1 or of the amino acids at the corresponding positions in a viral homologue of Nsp13, wherein the inhibitor binds to the backbone N of M378 with a hydrogen bond acceptor group; A379 of Nsp13 of SARSCoV2 according to SEQ ID NO: 1 or of the amino acids at the corresponding positions in a viral homologue of Nsp13, wherein the inhibitor binds to the backbone carbonyl oxygen of A379 with carbo halogens or hydrogen bond donating groups or to the backbone N of A379 with a hydrogen bond acceptor group; Y398 of Nsp13 of SARSCoV2 according to SEQ ID NO: 1 or of the amino acids at the corresponding positions in a viral homologue of Nsp13, wherein the inhibitor binds to the aromatic ring of Y398 via face-to-face or edge-to-face pi-pi interaction with aromatic carbo- or heterocyclic substituents or via cation-pi, polar-pi, or halogen-pi interactions with polar, charged, or carbo-halogen substituents; 1399 of Nsp13 of SARSCoV2 according to SEQ ID NO: 1 or of the amino acids at the corresponding positions in a viral homologue of Nsp13, wherein the inhibitor binds to the backbone carbonyl oxygen of 1399 with carbo halogen or hydrogen bond donating groups or to the backbone N of 1399 with a hydrogen bond acceptor group; G400 of Nsp13 of SARSCoV2 according to SEQ ID NO: 1 or of the amino acids at the corresponding positions in a viral homologue of Nsp13, wherein the inhibitor binds to the backbone N of G400 with a hydrogen bond acceptor group or to the backbone carbonyl oxygen of G400 with carbo halogen or hydrogen bond donating groups; D401 of Nsp13 of SARSCoV2 according to SEQ ID NO: 1 or of the amino acids at the corresponding positions in a viral homologue of Nsp13, wherein the inhibitor binds to the backbone carbonyl oxygen of D401 with carbo halogen or hydrogen bond donating groups; P406 of Nsp13 of SARSCoV2 according to SEQ ID NO: 1 or of the amino acids at the corresponding positions in a viral homologue of Nsp13, wherein the inhibitor binds to the backbone carbonyl oxygen of P406 with carbo halogen or hydrogen bond donating groups; and C426, wherein the inhibitor binds to the S of C426 with a carbo-halogen or the S of 426 forms a covalent bond to a “warhead” of the inhibitor.

The skilled person using a 3D model of Nsp13 or a homolog thereof or at least a 3D model of the binding pocket and in silico modelling can determine chemical groups that are capable of forming the above indicated bonds with the above indicated amino acids or chemical groups within the above indicated amino acids of the binding pocket (see also Example 1).

In one embodiment of the first aspect, the inhibitor is an organic molecule with a molecular weight within the range of 200-700 Da.

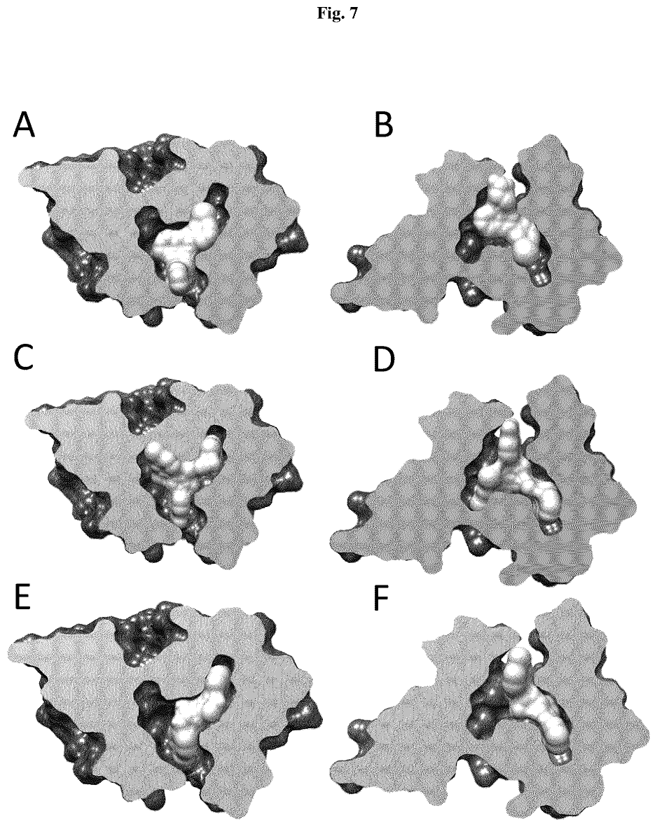

In one embodiment of the first aspect, the inhibitor has a structure and stereoelectronic properties complementary to the allosteric binding pocket. A structure of an inhibitor is considered complementary, if it can enter the binding pocket and fit into the tripartite structure of the binding pocket. An example of an inhibitor that has a structure and stereoelectronic properties complementary to the allosteric binding pocket and which thus fits into the binding pocket is shown in FIG. 7. The position of exemplary compounds that have a structure and stereoelectronic properties complementary to the allosteric binding pocket are also shown in FIG. 9. FIG. 11 is an 2D view of the binding pocket and shows how the three different compounds interact with amino acids in the binding pocket.

In one embodiment of the first aspect, the inhibitor consists of a central acyclic or cyclic core structure with 1-3 substituents that are independently directed at one, two or three of the channels selected from the group of entry channel, the left channel and a right channel of the tripartic allosteric binding pocket. The phrase a “substituent is directed at a channel” is used in the context of the present invention to characterize a substituent that protrudes into that channel and forms a non-covalent bond with at least one amino acid within the channel.

In a further embodiment of the first aspect, the inhibitor consists of a central heterocyclic or carbocyclic scaffold optionally substituted with 1-3 substituents that are independently directed at one, two or three of the channels selected from the group of entry channel, the left channel and a right channel of the tripartic allosteric binding pocket.

In a further embodiment of the first aspect, the inhibitor consists of a central heteroaromatic scaffold optionally substituted with 1-3 substituents that are independently directed at one, two or three of the channels selected from the group of entry channel, the left channel and a right channel of the tripartic allosteric binding pocket.

In a further embodiment of the first aspect, the inhibitor consists of a central heteroaromatic scaffold optionally substituted with 2 or 3 substituents, preferentially 3 substituents that are independently directed at one, two or three of the channels selected from the group of entry channel, the left channel and a right channel of the tripartic allosteric binding pocket.

In a further embodiment of the first aspect, the inhibitor consists of a central heteroaromatic scaffold comprising 1 to 3 5-, 6- or 7-membered rings, wherein 0 to 2 of the individual rings are selected from 5-, 6- or 7-membered carbocyclic rings and from 1 to 3 rings are selected from 5-, 6- or 7-membered heterocyclic rings, wherein each ring is annulated to at least one other ring and/or connected to at least one other ring via covalent bonds, and wherein the heteroaromatic scaffold is optionally substituted with 2 or 3 substituents, preferably 3 substituents that are independently directed at one, two or three of the channels selected from the group of entry channel, the left channel and a right channel of the tripartic allosteric binding pocket.

In a particular embodiment of the first aspect, the 5-, 6- or 7-membered heterocyclic ring is selected from the group consisting of imidazole, imidazoline, pyrazole, pyrazolone, pyrrole, 2-hydroxypyrrole, 1,2,3-triazole, thiophene, 1,2,4-thiadiazole, quinazoline, 1-quinoline, 3-quinoline, pyrrolopyridine, imidazopyridine, and pyrimidopyrimidine.

In a particular embodiment of the first aspect or in a second aspect, the inhibitor of the invention has a structure according to formula (I):

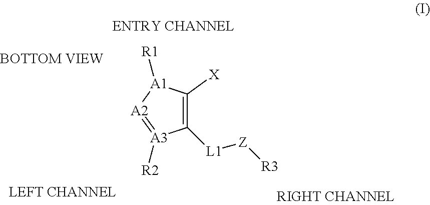

wherein
A1 and A3 are each independently selected from N or C;
A2 is selected from N, C or O;
X is H or OH or NH₂;
L1 is selected from the group consisting of C, CH, CH₂, O, N, and NH; or L1 is not present;
Z is a 5-, 6- or 7-membered carbo- or heterocycle substituted with R3, optionally further substituted, wherein R3 is connected to Z either via a bond or via a —CH₂— group;
R1 is H or a 5-, 6- or 7-membered carbo- or heterocycle, optionally substituted with one, two or three (preferably one) substituents that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, -Me, —CF₃, -Et, —OMe, and —SMe;
R2 is H or a 5-, 6- or 7-membered carbo- or heterocycle, optionally substituted with one, two or three (preferably one) lipophilic substituents that are independently from each other selected from the group consisting of —Br, —Cl, —F, —I, -Me, —CF₃, -Et, —OMe, and —SMe, wherein R2 is connected to A3 either via a bond or via a —CH₂— group;
R3 is H or a 5-, 6- or 7-membered carbo- or heterocycle, optionally substituted with one, two or three (preferably one) substituents that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, -Me, —CF₃, -Et, —OMe, and —SMe;
   or pharmaceutically acceptable salt, isomer, solvate, chemically protected form, or prodrug thereof, wherein R1, R2 and R3 preferably have a molecular shape and stereoelectronic properties complementary to the allosteric binding pocket.

In above formula (I) the “Entry Channel”, “Left Channel” and “Right Channel” are merely indicated to indicate the relative position that the inhibitor of the invention will occupy when bound to the binding pocket. The indicating of the position of the three channels aids the skilled person in selecting suitable substituents from the above indicated substituents that can bind to the amino acids within the respectively indicated part of the binding pocket. Thus, in a further embodiment of the first aspect R1 is selected in such that it binds to amino acids in the Entry Chanel, R2 is selected in such that it binds to amino acids in the Left Channel and R3 is selected in such that it binds to amino acids in the Right Channel.

In above embodiment it is preferred that
A1 is N substituted with R1.

In above embodiment it is preferred that
A2 is N.

In above embodiment it is preferred that
A3 is C substituted with R2, wherein R2 is connected to A3 either via a bond or via a —CH₂— group.

In above embodiment it is preferred that
L1 is CH═ or CH₂ connected to Z.

In above embodiment it is preferred that
Z is a 5-, 6-membered heterocycle substituted with R3, optionally further substituted, preferably with one or two ═O or —NH₂, more preferably with one or two ═O; wherein R3 is connected to Z either via a bond or via a —CH₂— group is a 5-membered heterocycle, preferably 5-membered heteroaryl substituted with R3, optionally further substituted, wherein R3 is connected to Z either via a bond or via a —CH₂— group, preferably via a —CH₂— group. Particularly preferred 5-membered heteroaryls are selected from the group consisting of furanyl, thiophenyl, oxazolyl, isoxazolyl, 1,2,3-oxadiazolyl, 1,2,4-oxadiazolyl, pyrrolyl, imidazolyl, pyrazolyl, 1,2,3-triazolyl, 1,2,4-triazolyl, thiazolyl, isothiazolyl, 1,2,3-thiadiazolyl, 1,2,4-thiadiazolyl, optionally further substituted, wherein R3 is connected to Z either via a bond or via a —CH₂— group. The 5 membered heteroaryl may be further substituted with one or two ═O or —NH₂, more preferably with one or two ═O. In a further preferred embodiment Z is a 5-membered N-heteroaryl, preferably imidazolyl, selected from pyrrolyl, imidazolyl, pyrazolyl, 1,2,3-triazolyl, 1,2,4-triazolyl, preferably optionally further substituted, preferably with one or two ═O or —NH₂, more preferably with one or two ═O. In the most preferred embodiment Z is imidazolidine-2,4-dionyl. wherein R3 is connected to the imidazolidine-2,4-dionyl at the N at position 3 via a —CH₂— group.

In above embodiment it is preferred that
R1 is a 6-membered aryl or a 5- or 6-membered heteroaryl, optionally substituted with one, two or three, preferably one substituent(s) that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, -Me, —CF₃, -Et, —OMe, and —SMe. More preferably R1 is a 6-membered aryl, optionally substituted with one, two or three, preferably one substituent(s) that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, -Me, —CF₃, -Et, —OMe, and —SMe.

In above embodiment it is preferred that
R2 is a 6-membered aryl or a 5- or 6-membered heteroaryl, optionally substituted with one, two or three, preferably one lipophilic substituent(s) that are independently from each other selected from the group consisting of —Br, —Cl, —F, —I, Me, —CF₃, Et, —OMe, and —SMe. More preferably R2 is a 6-membered aryl, optionally substituted with one, two or three, preferably one lipophilic substituent(s) that are independently from each other selected from the group consisting of —Br, —Cl, —F, —I, Me, —CF₃, Et, —OMe, and —SMe. If R2 is substituted once than the one lipophilic substituent(s) that is independently from each other selected from the group consisting of —Br, —Cl, —F, —I, Me, —CF₃, Et, —OMe, and —SMe is in para position. If R2 is substituted twice the two lipophilic substituent(s) that are independently from each other selected from the group consisting of —Br, —Cl, —F, —I, Me, —CF₃, Et, —OMe, and —SMe are in ortho and para position.

In above embodiment it is preferred that
R3 is a 6-membered aryl or a 5- or 6-membered heteroaryl, optionally substituted with one, two or three, preferably one substituent(s) that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe. More preferably R1 is a 6-membered aryl, optionally substituted with one, two or three, preferably one substituent(s) that are independently from each other selected from the group consisting of NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe.

In a further embodiment of the first aspect, or in a second aspect of the invention the inhibitor has a structure according to formula (I), wherein
A1 is N substituted with R1;
A2 is N; and
A3 is C substituted with R2, wherein R2 is connected to A3 either via a bond or via a —CH₂— group.

In above embodiment it is preferred that:
X is H or OH.

In above embodiment it is preferred that:
L1 is CH═ or CH₂ connected to Z.

In above embodiment it is preferred that:
Z is a 5- or 6-membered heterocycle substituted with R3, wherein R3 is connected to Z either via a bond or via a —CH₂— group.

In above embodiment it is preferred that:
R1 is a 6-membered aryl or a 5- or 6-membered heteroaryl, optionally substituted with one, two or three, preferably one substituent(s) that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe.

In above embodiment it is preferred that:
R2 is a 6-membered aryl or a 5- or 6-membered heteroaryl, optionally substituted with one, two or three, preferably one lipophilic substituent(s) that are independently from each other selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position.

In above embodiment it is preferred that: R3 is a 6-membered aryl or a 5- or 6-membered heteroaryl, optionally substituted with one, two or three, preferably one substituent(s) that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe.

In above embodiment it is preferred that:
X is H or OH; and
L1 is CH═ or CH₂ connected to Z.

In above embodiment it is preferred that:
X is H or OH;
L1 is CH═ or CH₂ connected to Z; and
Z is a 5- or 6-membered heterocycle substituted with R3, wherein R3 is connected to Z either via a bond or via a —CH₂— group.

In above embodiment it is preferred that:
X is H or OH;
L1 is CH═ or CH₂ connected to Z; and
R1 is a 6-membered aryl or a 5- or 6-membered heteroaryl, preferably 6-membered aryl or heteroaryl, more preferably 6-membered aryl, optionally substituted with one, two or three (preferably one) substituents that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe.

In above embodiment it is preferred that:
X is H or OH;
L1 is CH═ or CH₂ connected to Z; and
R2 is 6-membered aryl or a 5- or 6-membered heteroaryl, preferably 6-membered aryl or heteroaryl, more preferably 6-membered aryl, optionally substituted with one, two or three, preferably one lipophilic substituent(s) that are independently from each other selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position.

In above embodiment it is preferred that:
X is H or OH;
L1 is CH═ or CH₂ connected to Z; and
R3 is 6-membered aryl or a 5- or 6-membered heteroaryl, preferably 6-membered aryl or heteroaryl, more preferably 6-membered aryl, optionally substituted with one, two or three (preferably one) substituents that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe.

In above embodiment it is preferred that:
X is H or OH;
L1 is CH═ or CH₂ connected to Z;
Z is a 5- or 6-membered heterocycle substituted with R3, wherein R3 is connected to Z either via a bond or via a —CH₂— group; and
R1 is a 6-membered aryl or a 5- or 6-membered heteroaryl, preferably 6-membered aryl or heteroaryl, more preferably 6-membered aryl, optionally substituted with one, two or three (preferably one) substituents that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe;

In above embodiment it is preferred that:
X is H or OH;
L1 is CH═ or CH₂ connected to Z;
Z is a 5- or 6-membered heterocycle substituted with R3, wherein R3 is connected to Z either via a bond or via a —CH₂— group; and
R2 is a 6-membered aryl or a 5- or 6-membered heteroaryl, preferably 6-membered aryl or heteroaryl, more preferably 6-membered aryl, optionally substituted with one, two or three (preferably one) lipophilic substituents that are independently from each other selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position; and

In above embodiment it is preferred that:
X is H or OH;
L1 is CH═ or CH₂ connected to Z;
Z is a 5- or 6-membered heterocycle substituted with R3, wherein R3 is connected to Z either via a bond or via a —CH₂— group; and
R3 is a 6-membered aryl or a 5- or 6-membered heteroaryl, preferably 6-membered aryl or heteroaryl, more preferably 6-membered aryl, optionally substituted with one, two or three (preferably one) substituents that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe.

In above embodiment it is preferred that:
A1 is N substituted with R1;
A2 is N;
A3 is C substituted with R2, wherein R2 is connected to A3 either via a bond or via a —CH₂— group;
X is H or OH;
L1 is CH═ or CH₂ connected to Z;
Z is a 5- or 6-membered heterocycle substituted with R3, wherein R3 is connected to Z either via a bond or via a —CH₂— group;
R1 is a 6-membered aryl or a 5- or 6-membered heteroaryl, preferably 6-membered aryl or heteroaryl, more preferably 6-membered aryl, optionally substituted with one, two or three (preferably one) substituents that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, -Me, —CF₃, -Et, —OMe, and —SMe;
R2 is a 6-membered aryl or a 5- or 6-membered heteroaryl, preferably 6-membered aryl or heteroaryl, more preferably 6-membered aryl, optionally substituted with one, two or three (preferably one) lipophilic substituents that are independently from each other selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position; and
R3 is a 6-membered aryl or a 5- or 6-membered heteroaryl, preferably 6-membered aryl or heteroaryl, more preferably 6-membered aryl, optionally substituted with one, two or three (preferably one) substituents that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe.

In above embodiment it is preferred that:
A1 is N substituted with R1;
A2 is N;
A3 is C substituted with R2, wherein R2 is connected to A3 either via a bond or via a —CH₂— group;
X is H or OH;
L1 is CH═ or CH₂ connected to Z;
Z is a 5-membered heterocycle substituted with R3, wherein R3 is connected to Z either via a bond or via a —CH₂— group;
R1 is a 6-membered aryl or heteroaryl, preferably 6-membered aryl, optionally substituted with one or two, preferably one substituents that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe;
R2 is a 6-membered aryl or heteroaryl, preferably 6-membered aryl, optionally substituted with one or two lipophilic substituents that are independently from each other selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position; and
R3 is a 6-membered aryl or heteroaryl, preferably 6-membered aryl, optionally substituted with one or two (preferably one) substituents that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe.

In a further embodiment of the first aspect or in a second aspect of the invention, the inhibitor has a structure according to formula (I), wherein
A1 is N substituted with R₁;
A2 is N; and
A3 is C substituted with R₂, wherein R2 is connected to A3 either via a bond or via a —CH₂— group.

In above embodiment it is preferred that:
X is H or OH.

In above embodiment it is preferred that:
L1 is CH═ connected to Z.

In above embodiment it is preferred that:
Z is a 5-membered heterocycle, preferably a 5-membered heteroaryl, more preferably a 5-membered N-heteroaryl, substituted with R3, wherein R3 is connected to Z either via a bond or via a —CH₂— group, preferably via a —CH₂— group.

In above embodiment it is preferred that:
R1 is a 6-membered aryl or a 5- or 6-membered heteroaryl, preferably 6-membered aryl or heteroaryl, more preferably 6-membered aryl, optionally substituted with one, two or three, preferably one substituent(s) that are independently from each other selected from the group consisting of —NO₂, —CN, —OH, COOH, NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe.

In above embodiment it is preferred that:
R2 is a 6-membered aryl or a 5- or 6-membered heteroaryl, preferably 6-membered aryl or heteroaryl, more preferably 6-membered aryl, optionally substituted with one, two or three, preferably one lipophilic substituent(s) selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position.

In above embodiment it is preferred that:
R3 is a 6-membered aryl or a 5- or 6-membered heteroaryl, preferably 6-membered aryl or heteroaryl, more preferably 6-membered aryl, optionally substituted with one, two or three, preferably one substituent(s) that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe, wherein R3 is connected to Z via a —CH₂— group.

In above embodiment it is preferred that:
X is H or OH; and
L1 is CH═ connected to Z.

In above embodiment it is preferred that:
X is H or OH;
L1 is CH═ connected to Z; and
Z is a 5-membered heterocycle, preferably 5-membered N-heterocycle substituted with R3, wherein R3 is connected to Z either via a bond or via a —CH₂— group, preferably via a —CH₂— group.

In above embodiment it is preferred that:
X is H or OH;
L1 is CH═ connected to Z;
Z is a 5-membered heterocycle substituted with R3, wherein R3 is connected to Z either via a bond or via a —CH₂— group; and
R1 is a 6-membered aryl or a 5- or 6-membered heteroaryl, preferably 6-membered aryl or heteroaryl, more preferably 6-membered aryl, optionally substituted with one, two or three, preferably one substituent(s) that are independently from each other selected from the group consisting of —NO₂, —CN, —OH, COOH, NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe;

In above embodiment it is preferred that:
X is H or OH;
L1 is CH═ connected to Z;
Z is a 5-membered heterocycle, preferably 5-membered N-heterocycle substituted with R3, wherein R3 is connected to Z either via a bond or via a —CH₂— group; and
R2 is a 6-membered aryl or a 5- or 6-membered heteroaryl, preferably 6-membered aryl or heteroaryl, more preferably 6-membered aryl, optionally substituted with one, two or three, preferably one lipophilic substituent(s) selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position; and

In above embodiment it is preferred that:
X is H or OH;
L1 is CH═ connected to Z;
Z is a 5-membered heterocycle, preferably 5-membered N-heterocycle substituted with R3, wherein R3 is connected to Z either via a bond or via a —CH₂— group, preferably via a —CH₂— group; and
R3 is a 6-membered aryl or a 5- or 6-membered heteroaryl, preferably 6-membered aryl or heteroaryl, more preferably 6-membered aryl, optionally substituted with one, two or three, preferably one substituent(s) that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe, wherein R3 is connected to Z via a —CH₂— group.

In above embodiment it is preferred that:
A1 is N substituted with R₁;
A2 is N;
A3 is C substituted with R₂, wherein R2 is connected to A3 either via a bond or via a —CH₂— group;
X is H or OH;
L1 is CH═ connected to Z;
Z is a 5-membered heterocycle, preferably 5-membered N-heterocycle substituted with R3, wherein R3 is connected to Z either via a bond or via a —CH₂— group, preferably via a —CH₂— group;
R1 is a 6-membered aryl or a 5- or 6-membered heteroaryl, preferably 6-membered aryl or heteroaryl, more preferably 6-membered aryl, optionally substituted with one, two or three, preferably one substituent(s) that are independently from each other selected from the group consisting of —NO₂, —CN, —OH, COOH, NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe;
R2 is a 6-membered aryl or a 5- or 6-membered heteroaryl, preferably 6-membered aryl or heteroaryl, more preferably 6-membered aryl, optionally substituted with one, two or three, preferably one lipophilic substituent(s) selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position; and
R3 is a 6-membered aryl or a 5- or 6-membered heteroaryl, preferably 6-membered aryl or heteroaryl, more preferably 6-membered aryl, optionally substituted with one, two or three, preferably one substituent(s) that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe, wherein R3 is connected to Z via a —CH₂— group.

In above embodiment it is preferred that:
A1 is N substituted with R₁;
A2 is N;
A3 is C substituted with R₂, wherein R2 is connected to A3 either via a bond or via a —CH₂— group.

X is H or OH;
L1 is CH═ connected to Z;
Z is a 5-membered heterocycle substituted with R3, wherein R3 is connected to Z either via a bond or via a —CH₂— group, preferably via a —CH₂— group;
R1 is a 6-membered aryl or heteroaryl, more preferably 6-membered aryl, optionally substituted with one, two or three, preferably one substituent(s) that are independently from each other selected from the group consisting of —NO₂, —CN, —OH, COOH, NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe;
R2 is a 6-membered aryl or heteroaryl, more preferably 6-membered aryl, optionally substituted with one, two or three, preferably one lipophilic substituent(s) selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position; and
R3 is a 6-membered aryl or heteroaryl, more preferably 6-membered aryl, optionally substituted with one, two or three, preferably one substituent(s) that are independently from each other selected from the group consisting of —NO₂, —CN, —OH, —COOH, —NH—SO₂-alkyl (particularly —NH—SO₂—(C₁-C₆)alkyl), NH—CO-alkyl (particularly —NH—CO—(C₁-C₆)alkyl), —CONH₂, —CONH-alkyl (particularly —CONH—(C₁-C₆)alkyl), —Br, —Cl, —F, —I, Me, —CF₃, Et, —OMe, and —SMe, wherein R3 is connected to Z via a —CH₂— group.

In a further embodiment of the first aspect or in a second aspect of the invention, the inhibitor has a structure according to formula (I), wherein
A1 is N substituted with R₁;
A2 is N;
A3 is C substituted with R₂, wherein R2 is connected to A3 either via a bond or via a —CH₂— group;
X is OH;
L1 is CH═ connected to Z;
Z is a 5-membered heterocycle substituted with R3, wherein R3 is connected to Z either via a bond or via a —CH₂— group;
R1 is a 6-membered aryl or a 5- or 6-membered heteroaryl, preferably 6-membered aryl or heteroaryl, more preferably 6-membered aryl, optionally substituted with one, two or three, preferably one polar substituent(s) that are independently from each other selected from the group consisting of —NO₂, —CN, —OH, COOH, NH—SO₂-alkyl (particularly —NH—SO₂—(C₁-C₆)alkyl), NH—CO-alkyl (particularly —NH—CO—(C₁-C₆)alkyl), —CONH₂, and —CONH-alkyl (particularly —CONH—(C₁-C₆)alkyl);
R2 is a 6-membered aryl or a 5- or 6-membered heteroaryl, preferably 6-membered aryl or heteroaryl, more preferably 6-membered aryl, optionally substituted with one, two or three, preferably one lipophilic substituent(s) that are independently from each other selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position;
R3 is a 6-membered aryl or a 5- or 6-membered heteroaryl, preferably 6-membered aryl or heteroaryl, more preferably 6-membered aryl, optionally substituted with one, two or three, preferably one lipophilic substituent(s) that are independently from each other selected from the group consisting of —Br, —Cl, —F, —I, Me, —CF₃, Et, —OMe, and —SMe.

In a further embodiment of the first aspect or in a second aspect of the invention, the inhibitor has a structure according to formula (I), wherein
A1 is N substituted with R₁;
A2 is N;
A3 is C substituted with R₂, wherein R2 is connected to A3 either via a bond or via a —CH₂— group;
X is OH;
L1 is CH═ connected to Z;
Z is imidazolidine-2,4-dione, wherein R3 is connected to Z via a —CH₂— group at the 3 position of the imidazolidine-2,4-dione;
R1 is a 6-membered aryl or heteroaryl, preferably 6-membered aryl, optionally substituted with one, two or three, preferably one polar substituent(s) that are independently from each other selected from the group consisting of —NO₂, —CN, —OH, COOH, NH—SO₂-alkyl (particularly —NH—SO₂—(C₁-C₆)alkyl), NH—CO-alkyl (particularly —NH—CO—(C₁-C₆)alkyl), —CONH₂, and —CONH-alkyl (particularly —CONH—(C₁-C₆)alkyl);
R2 is 6-membered aryl or heteroaryl, preferably 6-membered aryl, optionally substituted with one, two or three, preferably one lipophilic substituent(s) that are independently from each other selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position;
R3 is 6-membered aryl or heteroaryl, preferably 6-membered aryl, optionally substituted with one, two or three, preferably one lipophilic substituent(s) that are independently from each other selected from the group consisting of —Br, —Cl, —F, —I, Me, —CF₃, Et, —OMe, and —SMe.

In a further embodiment of the first aspect or another aspect of the invention, the inhibitor has a structure according to formula (I), wherein
A1 is N substituted with R1;
A2 is N;
A3 is C substituted with R2, wherein R2 is connected to A3 either via a bond or via a —CH₂— group;
X OH;
L1 is CH═ connected to Z;
Z is imidazolidine-2,4-dione, wherein R3 is connected to Z via a —CH₂— group at the N at the 3 position of the imidazolidine-2,4-dione;
R1 is phenyl, optionally substituted with one, two, or three, preferably one polar moieties that are independently from each other selected from the group consisting of —NO₂, —CN, —OH, COOH, NH—SO₂-alkyl (particularly —NH—SO₂—(C₁-C₆)alkyl), NH—CO-alkyl (particularly —NH—CO—(C₁-C₆)alkyl), —CONH₂, and —CONH-alkyl (particularly —CONH—(C₁-C₆)alkyl);
R2 is any phenyl or benzyl (preferably phenyl) substituted with one, two, or three (preferably one) lipophilic substituents that are independently from each other selected from the group consisting of —Br, —Cl, —F, —I, Me, —CF₃, Et, —OMe, and —SMe, if R2 is substituted once than the one lipophilic substituent(s) that is independently from each other selected from the group consisting of —Br, —Cl, —F, —I, Me, —CF₃, Et, —OMe, and —SMe is preferably in para position, and if R2 is substituted twice the two lipophilic substituent(s) that are independently from each other selected from the group consisting of —Br, —Cl, —F, —I, Me, —CF₃, Et, —OMe, and —SMe are preferably in ortho and para position;
R3 is phenyl substituted with one, two, or three (preferably one) lipophilic substituents that are independently from each other selected from the group consisting of —Br, —Cl, —F, —I, Me, —CF₃, Et, —OMe, and —SMe.

In a further embodiment of the first aspect or in a second aspect of the invention, the inhibitor has a structure according to formula (I), wherein
A1 is N substituted with R₁;
A2 is N;
A3 is C substituted with R2, wherein R2 is connected to A3 either via a bond or via a —CH₂— group;
X is OH;
L1 is CH═ connected to Z;
Z is imidazolidine-2,4-dione, wherein R3 is connected to Z via a —CH₂— group at the N at the 3 position of the imidazolidine-2,4-dione;
R1 is phenyl, 4-nitrophenyl, 2,6-dichloro-4-bromo-phenyl, 4-fluorophenyl, 4-bromophenyl, 2,6-dichlorophenyl, 2-methylphenyl, 4-methylphenyl, 2,3-dimethylphenyl, or 4-methoxyphenyl;
R2 is 2,4-dichlorophenyl, 4-chlorophenyl, phenyl, benzyl, 3-pyridine, 2-methylbenzyl, 4-fluorobenzyl, 4-chlorobenzyl, 2-methoxyphenyl, or 3-methoxyphenyl; and
R3 is 4-fluorophenyl, 2-fluorophenyl, 3-fluorophenyl, 2,4-difluorophenyl, 2-methylphenyl, 3-methylphenyl, 4-methylphenyl, 2-chlorophenyl, 3-chlorophenyl, 3-pyridine, 3-methoxyphenyl, 4-nitrophenyl, 3-nitrophenyl, 4-trifluoromethyl-phenyl, or 4-cyanophenyl.

In a further particular embodiment of the first aspect or in a second aspect of the invention, the inhibitor has a structure according to formula (II):

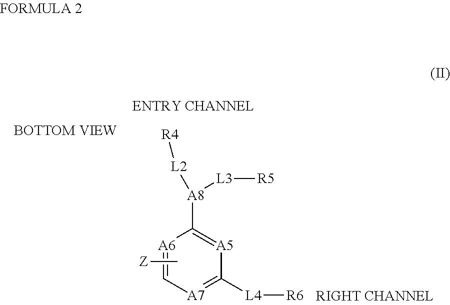

wherein
A5 and A8 are each independently selected from N or CH;
A6 is selected from N or CH, or when A6 takes part in the annulated carbo- or heterocycle Z, then A6 is C;
A7 is selected from N or CH, or when A7 takes part in the annulated carbo- or heterocycle Z, then A7 is C;
L2 is selected from the group consisting of —CH₂—R4, —CF₂—R4, —CH₂—CH₂—R4, —CH₂—CH₂—CH₂—R4, —O—R4, —NH—R4, —N═R4;
L3 is selected from the group consisting of CH₂—R5, —CF₂—R5, —CH₂—CH₂—R5, —CH₂—COH₂—R5, —CH═CH—R5, —CH₂—CF₂—R5, —CH₂—CH₂—CH₂—R5, —O—R5, —NH—R5, —N═R5; or
L2 and L3 together with the A8 to which they are connected form a 5- or 6-membered heterocycle substituted by R4 and/or R5;
L4 is CH₂, —CF₂—, CH₂—CH₂, CH₂—CH₂—CH₂, O, N, and NH, or is absent;
Z is a 5-, 6- or 7-membered carbo- or heterocycle, optionally substituted with one, two, or three, preferably one substituent(s) selected from the group consisting of —Br, —Cl, —F, —I, —OH, Me, —CF₃, Et, —OMe, —SMe, and —NO₂;
and can be annulated to the central core or connected via a covalent bond R4 is 5-, 6- or 7-membered carbo- or heterocycle, optionally substituted with one, two, or three, preferably one lipophilic substituent(s) selected from the group consisting of —Br, —Cl, —F, —I, —CF₃, Me, Et, —OMe, and —SMe, or R4 is hydrogen, methyl, or COOH;
R5 is a 5-, 6-, 7-, 8-, 9- or 10-membered carbo- or heterocycle, optionally substituted with one, two, or three (preferably one) lipophilic substituents selected from the group consisting of —Br, —Cl, —F, —I, Me, —CF₃, Et, —OMe, and —SMe;
R6 is a 5-, 6- or 7-membered carbo- or heterocycle, optionally substituted with one, two, or three (preferably one) substituents selected from the group consisting of —Br, —Cl, —F, —I, —OH, —NO₂, Me, —CF₃, Et, —OMe, and —SMe;
or R6 is H;
or
when A7 takes part in the annulated carbo- or heterocycle Z then A5 and A6 are independently selected from —N or —CH and A8 is selected from —N, —CH, —CH₂—N, —CH₂—CH, or —NH—CH;
or pharmaceutically acceptable salt, isomer, solvate, chemically protected form, or prodrug thereof, wherein R4, R5, and R6 preferably have a molecular shape and stereoelectronic properties complementary to the allosteric binding pocket.

The above definitions of linkers L2 and L3 in the context of formula (II) contain the substituents R4 and R5, respectively. The recitation of substituents R4 and R5 within the linker definitions does not mean that R4 and R5 are present two times in the resulting compound according to formula (II). R4 and R5 are additionally listed in the definitions of L2 and L3 so as to clarify the orientation of the linker from A8 to R4 or from A8 to R5, respectively.

In above formula (II) the “Entry Channel”, and “Right Channel” are merely indicated to indicate the relative position that the inhibitor of the invention will occupy when bound to the binding pocket. The indicating of the position of the three channels aids the skilled person in selecting suitable substituents from the above indicated substituents that can bind to the amino acids within the respectively indicated part of the binding pocket. Thus, in a further embodiment of the first aspect R4 and R5 is selected in such that it binds to amino acids in the Entry Chanel, R6 is selected in such that it binds to amino acids in the Right Channel.

In above embodiment it is preferred that:
A5 and A6 are N; and
A7 takes part in the annulated carbo- or heterocycle, preferably carbocycle Z.

In above embodiment it is preferred that:
A5 and A6 are N;
A7 takes part in the annulated carbo- or heterocycle, preferably carbocycle Z; and
A8 is —CH₂—N, —CH₂—CH, or —NH—CH, preferably —NH—CH.

In above embodiment it is preferred that:
A5 is N and A8 is —N or —CH.

In above embodiment it is preferred that:
A5 is N and A8 is —N or —CH; and
A6 takes part in the annulated carbo- or heterocycle, preferably carbocycle Z.

In above embodiment it is preferred that:
L2 is selected from the group consisting of —CH₂—R4, —CH₂—CH₂—R4, —CH₂—CH₂—CH₂—R4.

In above embodiment it is preferred that:
L2 is selected from the group consisting of —CH₂—R4, —CF₂—R4, —CH₂—CH₂—R4, —CH₂—CH₂—CH₂—R4, —NH—R4; and
R4 is 6-membered aryl or 5-, 6- or 7-membered heteroaryl, preferably 5- or 6-membered heteroaryl, optionally substituted with one, two, or three, preferably one lipophilic substituent(s) selected from the group consisting of —Br, —Cl, —F, —I, —CF₃, Me, Et, —OMe, and —SMe, or R4 is hydrogen, methyl, or COOH.

In above embodiment it is preferred that:
L3 is selected from the group consisting of —CH₂—R5, —CH₂—CH₂—R5, —CH₂—CF₂—R5;

In above embodiment it is preferred that:
L3 is selected from the group consisting of —CH₂—R5, —CH₂—CH₂—R5, —CH₂—CF₂—R5; and
R5 is a 5-, 6-, 7-, 8-, 9- or 10-membered carbo- or heterocycle, optionally substituted with one, two, or three (preferably one) lipophilic substituents selected from the group consisting of —Br, —Cl, —F, —I, Me, —CF₃, Et, —OMe, and —SMe.

In above embodiment it is preferred that:
L2 and L3 together with the A8 to which they are connected form a 5- or 6-membered heterocycle substituted by R4 and R5.

In above embodiment it is preferred that:
L2 and L3 together with the A8 to which they are connected form a 5- or 6-membered heterocycle substituted by R4 and R5;
R4 is hydrogen, methyl, or COOH;
R5 is a 5-, 6-, 7-membered carbo- or heterocycle, preferably phenyl or 5- or 6-membered heteroaryl, optionally substituted with one, two, or three, preferably one lipophilic substituent(s) selected from the group consisting of —Br, —Cl, —F, —I, Me, —CF₃, Et, —OMe, and —SMe.

In above embodiment it is preferred that:
L4 is absent.

In above embodiment it is preferred that:
R4 is hydrogen, methyl, COOH or tetrazolyl.

In above embodiment it is preferred that:
L2 is —CH₂—R4, —CH₂—CH₂—R4, —CH₂—CH₂—CH₂—R4; and
R4 is hydrogen, methyl, COOH or tetrazolyl.

In above embodiment it is preferred that:
R5 is any 5-, 6-7-, 8-, 9-, or 10-membered carbo- or heterocycle, optionally substituted with one, two, or three, preferably one lipophilic substituent(s) selected from the group consisting of —Br, —Cl, —F, —I, —CF₃, Me, Et, —OMe, and —SMe.

In above embodiment it is preferred that:
R5 is C₅ to C₇-cycloalkyl, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one lipophilic substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one lipophilic substituent(s) selected from the group consisting of —Br, —Cl, —F, —I, Me, —CF₃, Et, —OMe, and —SMe, more preferably unsubstituted phenyl or adamantyl substituted with one, or two lipophilic substituent(s) selected from the group consisting of —Br, —Cl, —F, —I, Me, —CF₃, Et, —OMe, and —SMe

In above embodiment it is preferred that:
L3 is selected from the group consisting of —CH₂—R5, —CH₂—CH₂—R5, —CH₂—CF₂—R5; and
R5 is C₅ to C₇-cycloalkyl, i.e. C₅-, C₆- or C₇-cycloalkyl, C₆ to C₁₀-bicycloalkyl, i.e. C₆-, C₇-, C₅-, C₉- or C₁₀-bicycloalkyl, C₆ to C₁₀-spiroalkyl, i.e. C₆-, C₇—, C₅—, C₉- or C₁₀-spiroalkyl, phenyl, 5 or 6 membered heteroaryl, adamantyl, optionally substituted with one, two, or three, preferably one lipophilic substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one lipophilic substituent(s) selected from the group consisting of —Br, —Cl, —F, —I, Me, —CF₃, Et, —OMe, and —SMe, more preferably unsubstituted phenyl or adamantyl substituted with one, or two lipophilic substituent(s) selected from the group consisting of —Br, —Cl, —F, —I, Me, —CF₃, Et, —OMe, and —SMe

In above embodiment it is preferred that:
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe or R6 is H.

In above embodiment it is preferred that:
L4 is absent; and
R6 is a 5-, or 6-membered carbo- or heterocycle, preferably 6-membered carbo- or heterocycle, optionally substituted with one, two, or three (preferably one) substituents selected from the group consisting of —Br, —Cl, —F, —I, —OH, —NO₂, Me, —CF₃, Et, —OMe, and —SMe.

In above embodiment it is preferred that:
Z is a 6 membered aryl, or a 5-, 6-membered heteroaryl, preferably phenyl, optionally substituted with one, two, or three, preferably one substituent(s) selected from the group consisting of —Br, —Cl, —F, —I, —OH, Me, —CF₃, Et, —OMe, —SMe, and —NO₂.

In a further embodiment of the first aspect or in a second aspect of the invention, the inhibitor has a structure according to formula (II), wherein
A5 is N;
one of A6 and A7 is CH and the other one is N; or one of A6 and A7 is C and takes part in the annulated carbo- or heterocycle Z and the other one is N;
A8 is N or CH;
L2, L3, L4 are independently from each other selected from the group consisting of CH₂, —CF₂—, CH₂—CH₂, CH₂—CH₂—CH₂, 0, N, and NH, or are absent, or L2 and L3 together with the A8 to which they are connected form a 5- or 6-membered heterocycle substituted by R4 and R5;
Z is any 5-, 6- or 7-membered carbo- or heterocycle and can be annulated to the central core or connected via a covalent bond and optionally substituted with one, two, or three (preferably one) substituents selected from the group consisting of —Br, —Cl, —F, —I, —OH, Me, —CF₃, Et, —OMe, —SMe, and —NO₂;
R4 is any 5-, 6- or 7-membered carbo- or heterocycle, optionally substituted with one, two, or three (preferably one) lipophilic substituents selected from the group consisting of —Br, —Cl, —F, —I, —CF₃, Me, Et, —OMe, and —SMe,
   or R4 is hydrogen, methyl, or COOH;
R5 is a 5-, 6-7-, 8-, 9-, or 10-membered carbo- or heterocycle, optionally substituted with one, two, or three (preferably one) lipophilic substituents selected from the group consisting of —Br, —Cl, —F, —I, —CF₃, Me, Et, —OMe, and —SMe;
R6 is a 5-, 6- or 7-membered carbo- or heterocycle, optionally substituted with one, two, or three (preferably one) substituents selected from the group consisting of —Br, —Cl, —F, —I, —OH, —NO₂, Me, —CF₃, Et, —OMe, and —SMe,
   or R6 is H
or
when A7 takes part in the annulated carbo- or heterocycle Z then A5 and A6 are N and A8 is selected from —N, —CH, —CH₂—N, —CH₂—CH, or —NH—CH, preferably —CH₂—N, —CH₂—CH, or —NH—CH;
wherein R4, R5, and R6 preferably have a molecular shape and stereoelectronic properties complementary to the allosteric binding pocket.

In a further embodiment of the first aspect or in a second aspect of the invention, the inhibitor has a structure according to formula (II), wherein
A5 is N;
one of A6 and A7 is C and takes part in the annulated carbo- or heterocycle Z and the other one is N;
A8 is N;
L2 is CH₂—CH₂ and L3 is CH₂—CH₂ or CH₂—CF₂; or L2 and L3 together with the A8 to which they are connected form a 5- or 6-membered heterocycle (preferably piperidine or a pyrrolidine) substituted by R4 and R5;
L4 is absent;
Z is a 6-membered carbo- or heterocycle annulated to the central core, wherein Z is optionally substituted with one, two, or three (preferably one) substituents selected from the group consisting of —Br, —Cl, —F, —I, —OH, Me, —CF₃, Et, —OMe, —SMe, and NO₂;
R4 is COOH or CH₂N4;
R5 is a 5-, 6-, 7-, or 10-membered carbo- or heterocycle, optionally substituted with one, two, or three, preferably one) lipophilic substituents selected from the group consisting of —Br, —Cl, —F, —I, Me, —CF₃, Et, —OMe, and —SMe;
R6 is a 5- or 6-membered carbo- or heterocycle, optionally substituted with one, two, or three, preferably one substituent(s) selected from the group consisting of —Br, —Cl, —F, —I, —OH, —NO₂, Me, —CF₃, Et, —OMe, and —SMe,
   or R6 is H.

In a further embodiment of the first aspect or in a second aspect of the invention, the inhibitor has a structure according to formula (II), wherein each one of A5, A7 and A8 is N;
A6 is C and takes part in the annulated carbo- or heterocycle Z;
L2 is CH₂—CH₂—R4;
L3 is CH₂—CH₂—R4;
or L2 and L3 together with the A8 to which they are connected form a piperidine ring or a pyrrolidine ring, substituted by R4 and R5;
L4 is absent;
Z is phenyl or cyclohexyl annulated to the central core, wherein Z is optionally substituted with one, two, or three, preferably one substituent(s) selected from the group consisting of —Br, —Cl, —F, —OH, Me, —CF₃, —OMe, and —NO₂, R4 is COOH or tetrazolyl;
R5 is phenyl, cyclopentyl or adamantyl, optionally substituted with one, two, or three, preferably one lipophilic substituents selected from the group consisting of —Br, —CF₃, Me, —CH₂—CF₃, and —OMe;
R6 is a 6-membered carbo- or heterocycle, preferably phenyl or cyclohexyl; more preferably phenyl, optionally substituted with one, two, or three, preferably one substituent(s) selected from the group consisting of Br, —Cl, —F, Me, —CF₃, —OMe, and —NO₂.

In a further embodiment of the first aspect or in a second aspect of the invention, the inhibitor has a structure according to formula (IV):

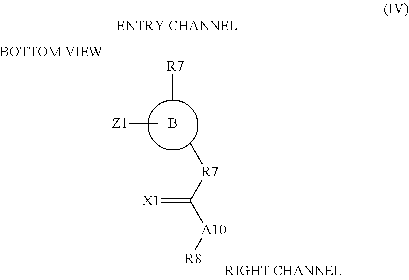

wherein
B is a 5-, 6-, or 7-membered heterocycle comprising 1, 2, or 3 heteroatoms selected from O, N, or S;
R7 is a 5-, 6- or 7-membered carbo- or heterocycle, optionally substituted with one, two, or three, preferably one lipophilic substituent(s) selected from the group consisting of —Br, —Cl, —F, —I, Me, Et, —OMe, and —SMe;
Z1 is selected from the group consisting of —Br, —Cl, —F, —I, —OH, —NH₂, Me, —CF₃, Et, —OMe, —SMe, and —NO₂; or Z1 is absent,
X1 is O or S, preferably O;
A9 is O, NH or CH₂;
A10 is O, NH or CH₂; and
R8 is a 5-, 6- or 7-membered carbo- or heterocycle, optionally substituted with one, two, or three (preferably one) lipophilic substituents selected from the group consisting of —Br, —Cl, —F, —I, Me, Et, —OMe, and —SMe;
or pharmaceutically acceptable salt, isomer, solvate, chemically protected form, or prodrug thereof,
wherein R7 and R8 preferably have a molecular shape and stereoelectronic properties complementary to the allosteric binding pocket.

In above formula (IV) the “Entry Channel”, “and “Right Channel” are merely indicated to indicate the relative position that the inhibitor of the invention will occupy when bound to the binding pocket. The indicating of the position of the two channels aids the skilled person in selecting suitable substituents from the above indicated substituents that can bind to the amino acids within the respectively indicated part of the binding pocket. Thus, in a further embodiment of the first aspect R7 is selected in such that it binds to amino acids in the Entry Chanel, and R8 is selected in such that it binds to amino acids in the Right Channel.

In above embodiment it is preferred that:
B is a 5-membered heterocycle comprising 1, 2, or 3 heteroatoms selected from O, N, or S, preferably comprising 3 heteroatoms selected from N, or S, more preferably 1,2,4-thiadiazaloyl. If B is 1,2,4-thiadiazaloyl it is preferred that A9 is substituted at the 5-position and R7 at the 3-positon of the 1,2,4-thiadiazaloyl.

In above embodiment it is preferred that:
X1 is O;
A9 is O or NH,
A10 is O or NH; and
B is a 5-membered heterocycle comprising 1, 2, or 3 heteroatoms selected from O, N, or S, preferably comprising 3 heteroatoms selected from N, or S, more preferably 1,2,4-thiadiazaloyl. If B is 1,2,4-thiadiazaloyl it is preferred that A9 is substituted at the 5-position and R7 at the 3-positon of the 1,2,4-thiadiazaloyl.

In above embodiment it is preferred that:
X1 is O or S (preferably O);
A9 is NH;
A10 is NH; and
B is a 5-membered heterocycle comprising 1, 2, or 3 heteroatoms selected from O, N, or S, preferably comprising 3 heteroatoms selected from N, or S, more preferably 1,2,4-thiadiazaloyl. If B is 1,2,4-thiadiazaloyl it is preferred that A9 is substituted at the 5-position and R7 at the 3-positon of the 1,2,4-thiadiazaloyl.

In above embodiment it is preferred that:
X1 is O;
A9 is O or NH,
A10 is O or NH;
B is a 5-membered heterocycle comprising 1, 2, or 3 heteroatoms selected from O, N, or S, preferably comprising 3 heteroatoms selected from N, or S, more preferably 1,2,4-thiadiazaloyl. If B is 1,2,4-thiadiazaloyl it is preferred that A9 is substituted at the 5-position and R7 at the 3-positon of the 1,2,4-thiadiazaloyl; and
R7 is 5- or 6-membered carbo- or heterocycle, preferably, 6-membered aryl or 5 or 6-membered heteroaryl, optionally substituted with one, two, or three, preferably one lipophilic substituent(s) selected from the group consisting of —Br, —Cl, —F, —I, Me, Et, —OMe, and —SMe, more preferably R7 is phenyl or 5-membered heteroaryl, preferably thiophenyl, optionally substituted with one, two, or three, preferably one lipophilic substituent(s) selected from the group consisting of —Br, —Cl, —F, —I, Me, Et, —OMe, and —SMe, more preferably unsubstituted phenyl or thiophenyl substituted with one or two, preferably one lipophilic substituent(s) selected from the group consisting of —Br, —Cl, —F, —I, Me, Et, —OMe, and —SMe.

In above embodiment it is preferred that:
X1 is O or S (preferably O);
A9 is NH;
A10 is NH;
B is a 5-membered heterocycle comprising 1, 2, or 3 heteroatoms selected from O, N, or S, preferably comprising 3 heteroatoms selected from N, or S, more preferably 1,2,4-thiadiazaloyl. If B is 1,2,4-thiadiazaloyl it is preferred that A9 is substituted at the 5-position and R7 at the 3-positon of the 1,2,4-thiadiazaloyl; and
R7 is 5- or 6-membered carbo- or heterocycle, preferably, 6-membered aryl or 5 or 6-membered heteroaryl, optionally substituted with one, two, or three, preferably one lipophilic substituent(s) selected from the group consisting of —Br, —Cl, —F, —I, Me, Et, —OMe, and —SMe, more preferably R7 is phenyl or 5-membered heteroaryl, preferably thiophenyl, optionally substituted with one, two, or three, preferably one lipophilic substituent(s) selected from the group consisting of —Br, —Cl, —F, —I, Me, Et, —OMe, and —SMe, more preferably unsubstituted phenyl or thiophenyl substituted with one or two, preferably one lipophilic substituent(s) selected from the group consisting of —Br, —Cl, —F, —I, Me, Et, —OMe, and —SMe.

In above embodiment it is preferred that:
R7 is 5- or 6-membered carbo- or heterocycle, preferably, 6-membered aryl or 5 or 6-membered heteroaryl, optionally substituted with one, two, or three, preferably one lipophilic substituent(s) selected from the group consisting of —Br, —Cl, —F, —I, Me, Et, —OMe, and —SMe, more preferably R7 is phenyl or 5-membered heteroaryl, preferably thiophenyl, optionally substituted with one, two, or three, preferably one lipophilic substituent(s) selected from the group consisting of —Br, —Cl, —F, —I, Me, Et, —OMe, and —SMe, more preferably unsubstituted phenyl or thiophenyl substituted with one or two, preferably one lipophilic substituent(s) selected from the group consisting of —Br, —Cl, —F, —I, Me, Et, —OMe, and —SMe.

In above embodiment it is preferred that:
B is a 5-membered heterocycle comprising 1, 2, or 3 heteroatoms selected from O, N, or S, preferably comprising 3 heteroatoms selected from N, or S, more preferably 1,2,4-thiadiazaloyl. If B is 1,2,4-thiadiazaloyl it is preferred that A9 is substituted at the 5-position and R7 at the 3-positon of the 1,2,4-thiadiazaloyl; and
R7 is phenyl or 5-membered heteroaryl, preferably thiophenyl, optionally substituted with one, two, or three, preferably one lipophilic substituent(s) selected from the group consisting of —Br, —Cl, —F, —I, Me, Et, —OMe, and —SMe, more preferably unsubstituted phenyl or thiophenyl substituted with one or two, preferably one lipophilic substituent(s) selected from the group consisting of —Br, —Cl, —F, —I, Me, Et, —OMe, and —SMe.

In a further embodiment of the first aspect or another aspect of the invention, the inhibitor has a structure according to formula (IV), wherein B is a 5-membered heterocycle comprising 1, 2, or 3 heteroatoms selected from O, N, or S.

In a further embodiment of the first aspect or in a second aspect of the invention, the inhibitor has a structure according to formula (IV), wherein
X1 is O;
A9 is O or NH; and
A10 is O or NH.

In a further embodiment of the first aspect or in a second aspect of the invention, the inhibitor has a structure according to formula (IV), wherein
X1 is O or S, preferably O;
A9 is NH; and
A10 is NH.

In a further particular embodiment of the first aspect or in a second aspect of the invention, the inhibitor has a structure according to formula (III):

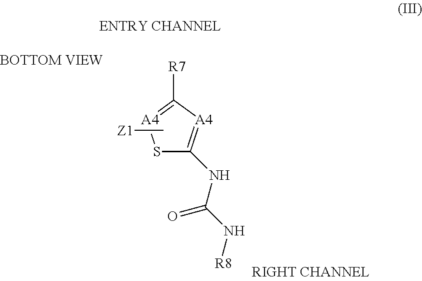

wherein
each A4 is independently from each other selected from N, NH, CH or CH₂;
Z1 is selected from the group consisting of —Br, —Cl, —F, —I, —OH, —NH₂, Me, —CF₃, Et, —OMe, —SMe, and —NO₂; or Z1 is absent;
R7 is any 5-, 6- or 7-membered carbo- or heterocycle, optionally substituted with one, two, or three (preferably one) lipophilic substituents selected from the group consisting of —Br, —Cl, —F, —I, Me, Et, —OMe, and —SMe;
R8 is any 5-, 6- or 7-membered carbo- or heterocycle, optionally substituted with one, two, or three (preferably one) lipophilic substituents selected from the group consisting of —Br, —Cl, —F, —I, Me, Et, —OMe, and —SMe;
or pharmaceutically acceptable salt, isomer, solvate, chemically protected form, or prodrug thereof,
wherein R7 and R8 preferably have a molecular shape and stereoelectronic properties complementary to the allosteric binding pocket.

In above formula (III) the “Entry Channel”, “and “Right Channel” are merely indicated to indicate the relative position that the inhibitor of the invention will occupy when bound to the binding pocket. The indicating of the position of the two channels aids the skilled person in selecting suitable substituents from the above indicated substituents that can bind to the amino acids within the respectively indicated part of the binding pocket. Thus, in a further embodiment of the first aspect R7 is selected in such that it binds to amino acids in the Entry Chanel, and R8 is selected in such that it binds to amino acids in the Right Channel.

In above embodiment it is preferred that:
R7 is 5- or 6-membered carbo- or heterocycle, preferably, 6-membered aryl or 5 or 6-membered heteroaryl, optionally substituted with one, two, or three, preferably one lipophilic substituent(s) selected from the group consisting of —Br, —Cl, —F, —I, Me, Et, —OMe, and —SMe, more preferably R7 is phenyl or 5-membered heteroaryl, preferably thiophenyl, optionally substituted with one, two, or three, preferably one lipophilic substituent(s) selected from the group consisting of —Br, —Cl, —F, —I, Me, Et, —OMe, and —SMe, more preferably unsubstituted phenyl or thiophenyl substituted with one or two, preferably one lipophilic substituent(s) selected from the group consisting of —Br, —Cl, —F, —I, Me, Et, —OMe, and —SMe.

In above embodiment it is preferred that:
A4 is N; and
R7 is 5- or 6-membered carbo- or heterocycle, preferably, 6-membered aryl or 5 or 6-membered heteroaryl, optionally substituted with one, two, or three, preferably one lipophilic substituent(s) selected from the group consisting of —Br, —Cl, —F, —I, Me, Et, —OMe, and —SMe, more preferably R7 is phenyl or 5-membered heteroaryl, preferably thiophenyl, optionally substituted with one, two, or three, preferably one lipophilic substituent(s) selected from the group consisting of —Br, —Cl, —F, —I, Me, Et, —OMe, and —SMe, more preferably unsubstituted phenyl or thiophenyl substituted with one or two, preferably one lipophilic substituent(s) selected from the group consisting of —Br, —Cl, —F, —I, Me, Et, —OMe, and —SMe.

In above embodiment it is preferred that:
Z1 is absent;
A4 is N; and
R7 is 5- or 6-membered carbo- or heterocycle, preferably, 6-membered aryl or 5 or 6-membered heteroaryl, optionally substituted with one, two, or three, preferably one lipophilic substituent(s) selected from the group consisting of —Br, —Cl, —F, —I, Me, Et, —OMe, and —SMe, more preferably R7 is phenyl or 5-membered heteroaryl, preferably thiophenyl, optionally substituted with one, two, or three, preferably one lipophilic substituent(s) selected from the group consisting of —Br, —Cl, —F, —I, Me, Et, —OMe, and —SMe, more preferably unsubstituted phenyl or thiophenyl substituted with one or two, preferably one lipophilic substituent(s) selected from the group consisting of —Br, —Cl, —F, —I, Me, Et, —OMe, and —SMe.

In above embodiment it is preferred that:
R8 is cyclohexyl, piperidinyl, hexahydropyridazinyl, hexahydropyrimidinyl, or piperazinyl, optionally substituted with one, two, or three (preferably one) lipophilic substituents selected from the group consisting of —Br, —Cl, —F, —I, Me, Et, —OMe, and —SMe.

In above embodiment it is preferred that:
Z1 is absent;
A4 is N; and
R8 is cyclohexyl, piperidinyl, hexahydropyridazinyl, hexahydropyrimidinyl, or piperazinyl, optionally substituted with one, two, or three, preferably one lipophilic substituent(s) selected from the group consisting of —Br, —Cl, —F, —I, Me, Et, —OMe, and —SMe.

In a further embodiment of the first aspect or in a second aspect of the invention, the inhibitor has a structure according to formula (III), wherein
each A4 is N or CH,
Z1 is selected from the group consisting of —Br, —Cl, —F, —I, —OH, —NH₂, Me, —CF₃, Et, —OMe, —SMe, and —NO₂; or Z1 is absent;
R7 is any 5-, 6- or 7-membered carbo- or heterocycle, optionally substituted with one, two, or three (preferably one) lipophilic substituents selected from the group consisting of —Br, —Cl, —F, —I, Me, Et, —OMe, and —SMe; and
R8 is any 5-, 6- or 7-membered carbo- or heterocycle, optionally substituted with one, two, or three, preferably one lipophilic substituent(s) selected from the group consisting of —Br, —Cl, —F, —I, Me, Et, —OMe, and —SMe.

In a further embodiment of the first aspect or in a second aspect of the invention, the inhibitor has a structure according to formula (III), wherein
each A4 is N,
Z1 is selected from the group consisting of —Br, —Cl, —F, —I, —OH, —NH₂, Me, —CF₃, Et, —OMe, —SMe, and —NO₂; or Z1 is absent;
R8 is cyclohexyl, piperidinyl, hexahydropyridazinyl, hexahydropyrimidinyl, or piperazinyl, optionally substituted with one, two, or three, preferably one lipophilic substituent(s) selected from the group consisting of —Br, —Cl, —F, —I, Me, Et, —OMe, and —SMe; and
R7 is a 5- or 6-membered carbo- or heterocycle (preferably 5- or 6-membered aryl or heteroaryl group), optionally substituted with a methyl group or an ethyl group.

In a further embodiment of the first aspect or in a second aspect of the invention, the inhibitor has a structure according to formula (III), wherein each A4 is N,
Z1 is absent;
R8 is methylpiperidinyl, preferably 1-methylpiperidinyl;
R7 is phenyl, optionally substituted with methyl or ethyl, or thiophene, optionally substituted with methyl or ethyl; preferably R7 is phenyl or methyl-substituted thiophenyl.

In a particular preferred embodiment of the first aspect or of the second aspect of the invention, the inhibitor is selected from the group consisting of

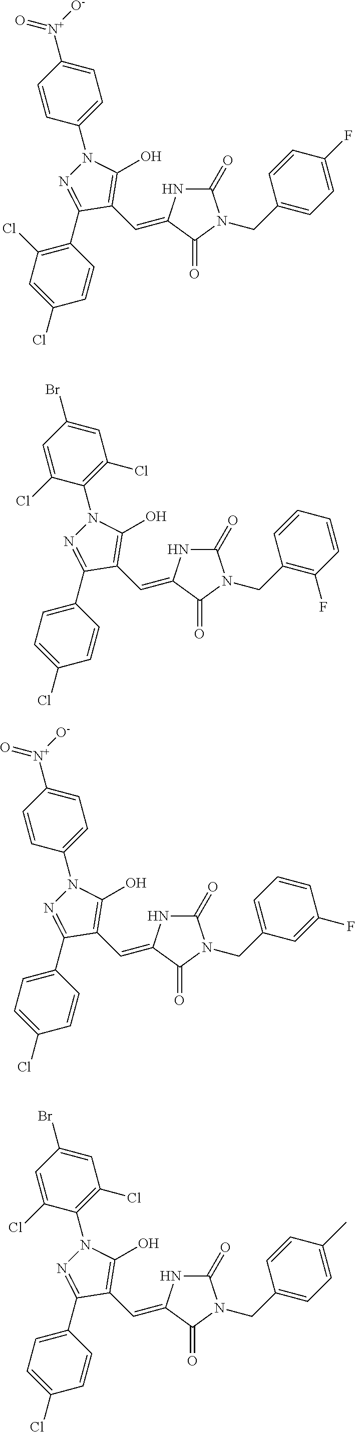

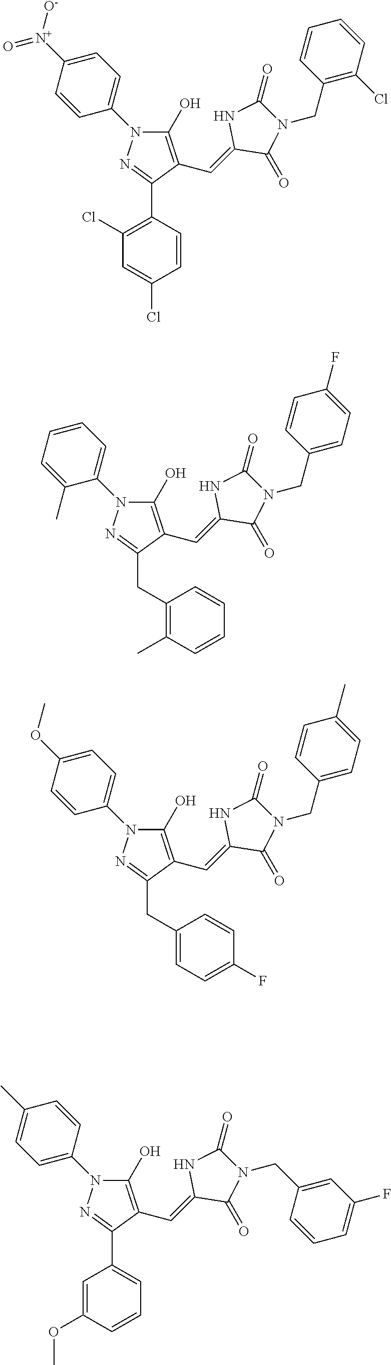 [SMILES: O=C1N/C(=C\c2c(-c3ccc(Cl)cc3Cl)nn(-c3ccc([N+](=O)[O-])cc3)c2O)C(=O)N1Cc1ccccc1Cl ; Cc1ccccc1Cc1nn(-c2ccccc2C)c(O)c1/C=C1\NC(=O)N(Cc2ccc(F)cc2)C1=O ; COc1ccc(-n2nc(Cc3ccc(F)cc3)c(/C=C3\NC(=O)N(Cc4ccc(C)cc4)C3=O)c2O)cc1 ; COc1cccc(-c2nn(-c3ccc(C)cc3)c(O)c2/C=C2\NC(=O)N(Cc3cccc(F)c3)C2=O)c1]

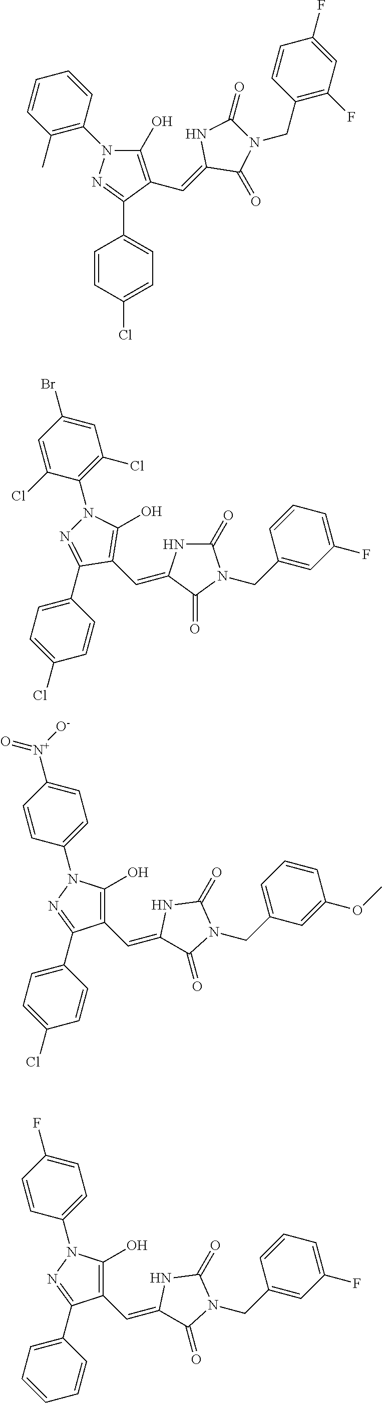

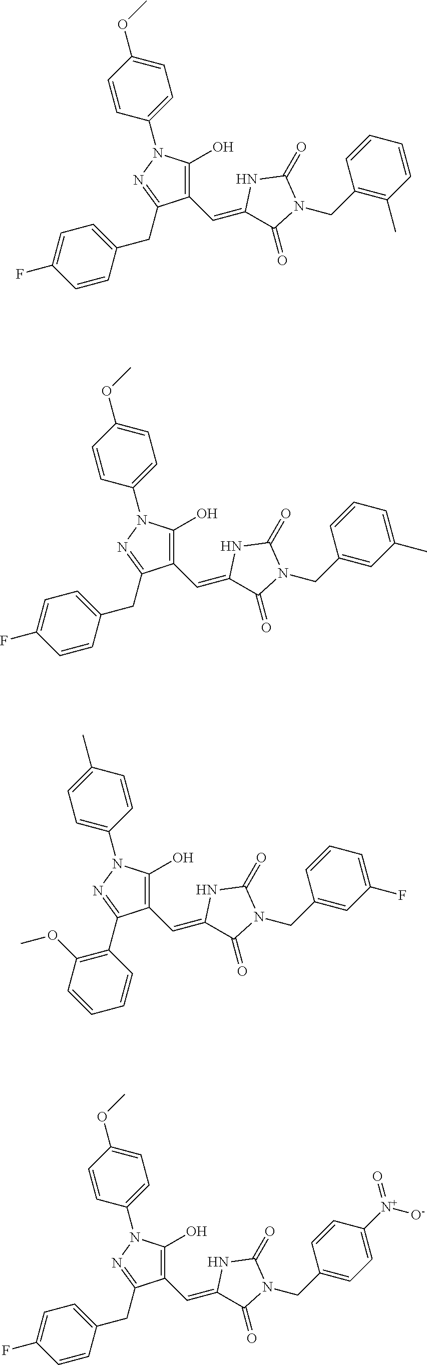

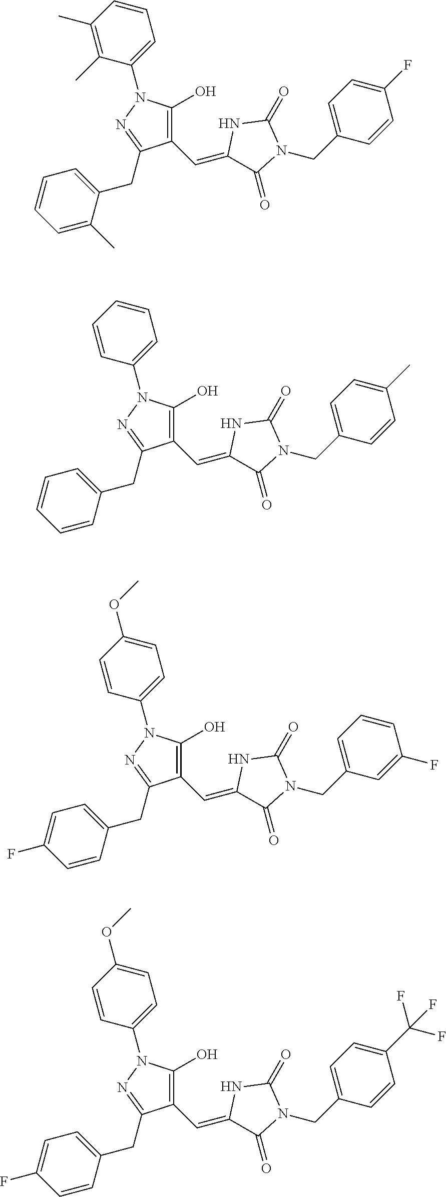 [SMILES: Cc1ccccc1Cc1nn(-c2cccc(C)c2C)c(O)c1/C=C1\NC(=O)N(Cc2ccc(F)cc2)C1=O ; Cc1ccc(CN2C(=O)N/C(=C\c3c(Cc4ccccc4)nn(-c4ccccc4)c3O)C2=O)cc1 ; COc1ccc(-n2nc(Cc3ccc(F)cc3)c(/C=C3\NC(=O)N(Cc4cccc(F)c4)C3=O)c2O)cc1 ; COc1ccc(-n2nc(Cc3ccc(F)cc3)c(/C=C3\NC(=O)N(Cc4ccc(C(F)(F)F)cc4)C3=O)c2O)cc1]

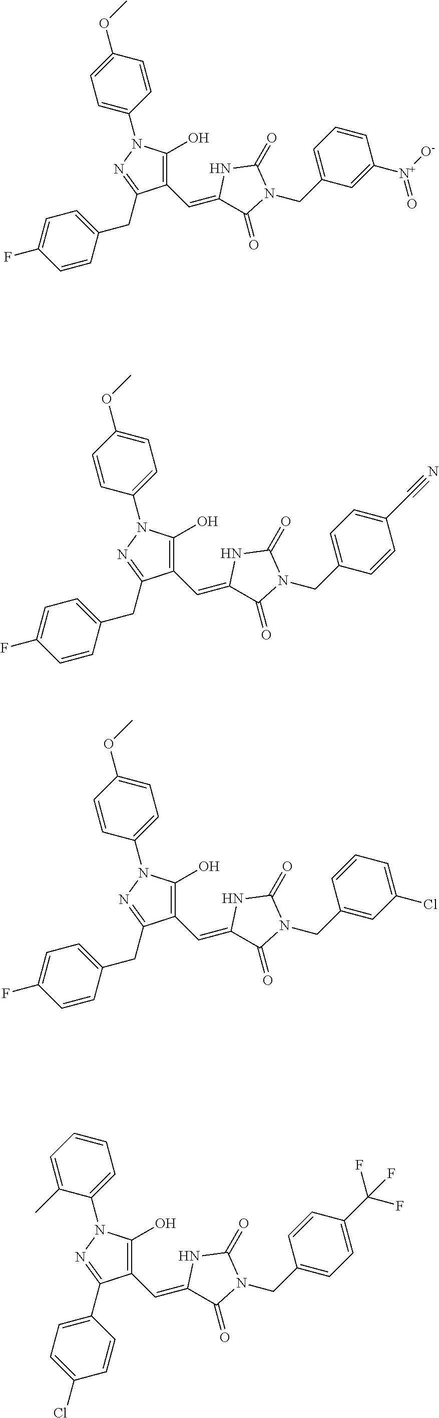 [SMILES: COc1ccc(-n2nc(Cc3ccc(F)cc3)c(/C=C3\NC(=O)N(Cc4cccc([N+](=O)[O-])c4)C3=O)c2O)cc1 ; COc1ccc(-n2nc(Cc3ccc(F)cc3)c(/C=C3\NC(=O)N(Cc4ccc(C#N)cc4)C3=O)c2O)cc1 ; COc1ccc(-n2nc(Cc3ccc(F)cc3)c(/C=C3\NC(=O)N(Cc4cccc(Cl)c4)C3=O)c2O)cc1 ; Cc1ccccc1-n1nc(-c2ccc(Cl)cc2)c(/C=C2\NC(=O)N(Cc3ccc(C(F)(F)F)cc3)C2=O)c1O]

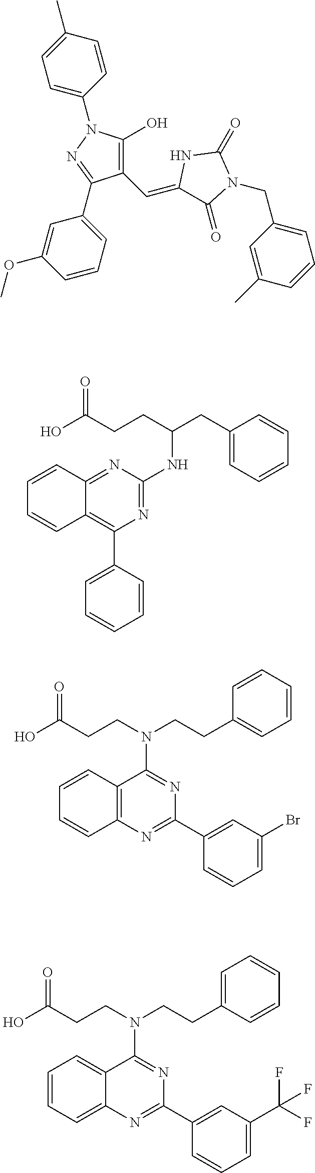 [SMILES: COc1cccc(-c2nn(-c3ccc(C)cc3)c(O)c2/C=C2\NC(=O)N(Cc3cccc(C)c3)C2=O)c1 ; O=C(O)CCC(Cc1ccccc1)Nc1nc(-c2ccccc2)c2ccccc2n1 ; O=C(O)CCN(CCc1ccccc1)c1nc(-c2cccc(Br)c2)nc2ccccc12 ; O=C(O)CCN(CCc1ccccc1)c1nc(-c2cccc(C(F)(F)F)c2)nc2ccccc12]

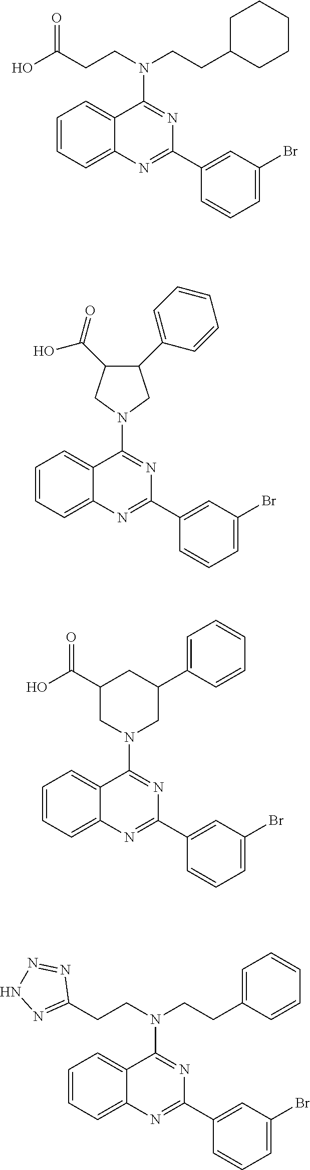 [SMILES: O=C(O)CCN(CCC1CCCCC1)c1nc(-c2cccc(Br)c2)nc2ccccc12 ; O=C(O)C1CN(c2nc(-c3cccc(Br)c3)nc3ccccc23)CC1c1ccccc1 ; O=C(O)C1CC(c2ccccc2)CN(c2nc(-c3cccc(Br)c3)nc3ccccc23)C1 ; Brc1cccc(-c2nc(N(CCc3ccccc3)CCc3nn[nH]n3)c3ccccc3n2)c1]

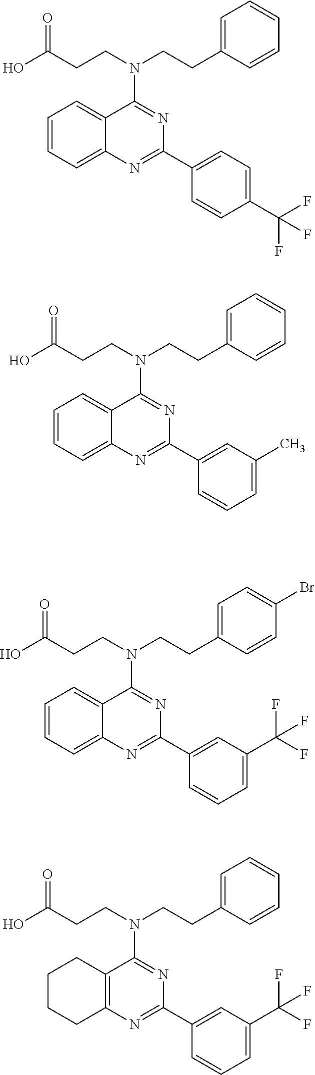 [SMILES: O=C(O)CCN(CCc1ccccc1)c1nc(-c2ccc(C(F)(F)F)cc2)nc2ccccc12 ; Cc1cccc(-c2nc(N(CCC(=O)O)CCc3ccccc3)c3ccccc3n2)c1 ; O=C(O)CCN(CCc1ccc(Br)cc1)c1nc(-c2cccc(C(F)(F)F)c2)nc2ccccc12 ; O=C(O)CCN(CCc1ccccc1)c1nc(-c2cccc(C(F)(F)F)c2)nc2c1CCCC2]

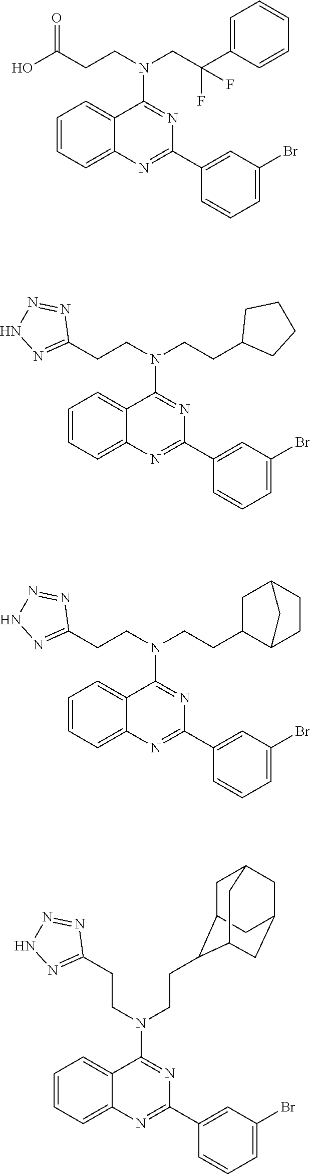 [SMILES: O=C(O)CCN(CC(F)(F)c1ccccc1)c1nc(-c2cccc(Br)c2)nc2ccccc12 ; Brc1cccc(-c2nc(N(CCc3nn[nH]n3)CCC3CCCC3)c3ccccc3n2)c1 ; Brc1cccc(-c2nc(N(CCc3nn[nH]n3)CCC3CC4CCC3C4)c3ccccc3n2)c1 ; Brc1cccc(-c2nc(N(CCc3nn[nH]n3)CCC3C4CC5CC(C4)CC3C5)c3ccccc3n2)c1]

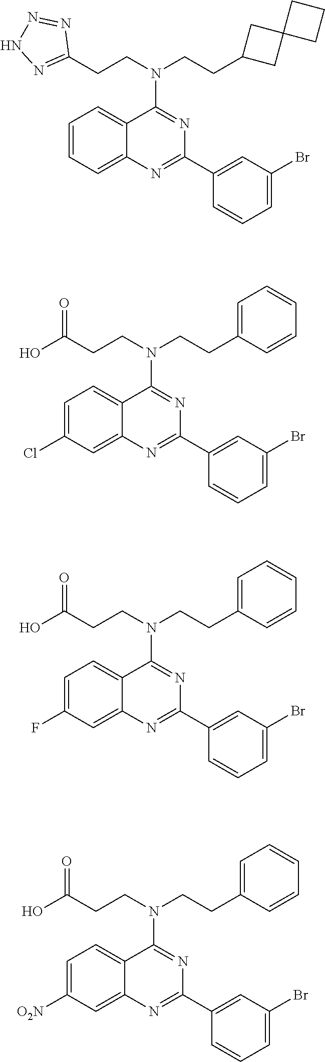 [SMILES: Brc1cccc(-c2nc(N(CCc3nn[nH]n3)CCC3CC4(CCC4)C3)c3ccccc3n2)c1 ; O=C(O)CCN(CCc1ccccc1)c1nc(-c2cccc(Br)c2)nc2cc(Cl)ccc12 ; O=C(O)CCN(CCc1ccccc1)c1nc(-c2cccc(Br)c2)nc2cc(F)ccc12 ; O=C(O)CCN(CCc1ccccc1)c1nc(-c2cccc(Br)c2)nc2cc([N+](=O)[O-])ccc12]

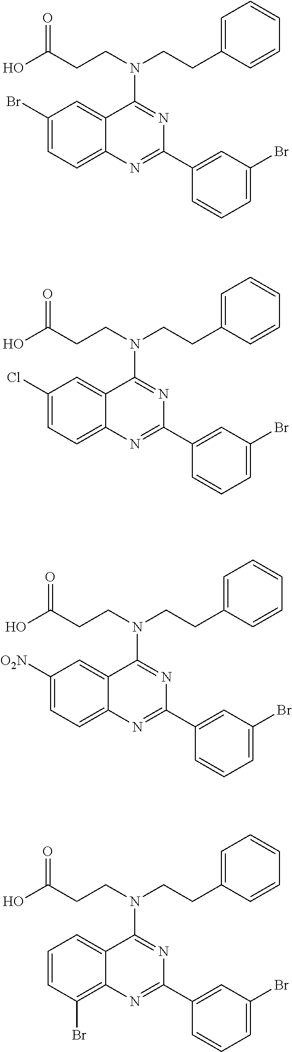 [SMILES: O=C(O)CCN(CCc1ccccc1)c1nc(-c2cccc(Br)c2)nc2ccc(Br)cc12 ; O=C(O)CCN(CCc1ccccc1)c1nc(-c2cccc(Br)c2)nc2ccc(Cl)cc12 ; O=C(O)CCN(CCc1ccccc1)c1nc(-c2cccc(Br)c2)nc2ccc([N+](=O)[O-])cc12 ; O=C(O)CCN(CCc1ccccc1)c1nc(-c2cccc(Br)c2)nc2c(Br)cccc12]

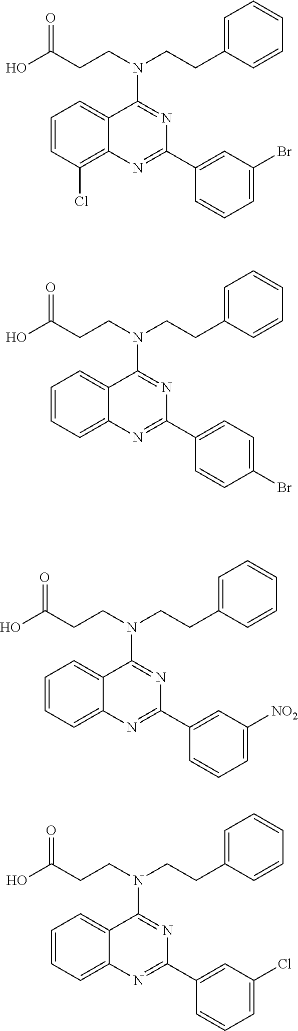 [SMILES: O=C(O)CCN(CCc1ccccc1)c1nc(-c2cccc(Br)c2)nc2c(Cl)cccc12 ; O=C(O)CCN(CCc1ccccc1)c1nc(-c2ccc(Br)cc2)nc2ccccc12 ; O=C(O)CCN(CCc1ccccc1)c1nc(-c2cccc([N+](=O)[O-])c2)nc2ccccc12 ; O=C(O)CCN(CCc1ccccc1)c1nc(-c2cccc(Cl)c2)nc2ccccc12]

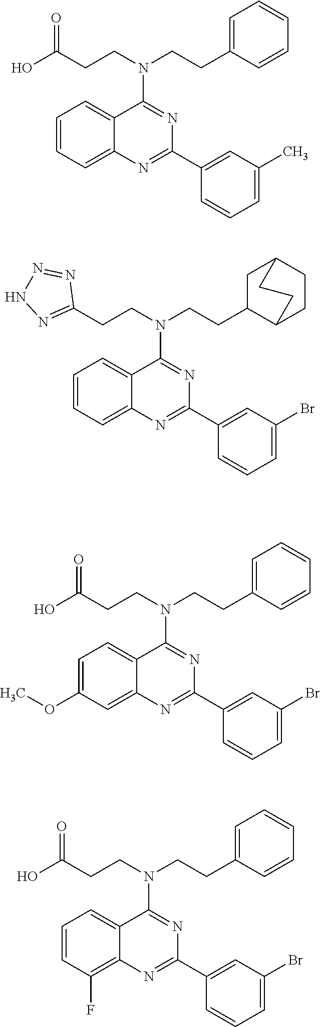 [SMILES: Cc1cccc(-c2nc(N(CCC(=O)O)CCc3ccccc3)c3ccccc3n2)c1 ; Brc1cccc(-c2nc(N(CCc3nn[nH]n3)CCC3CC4CCC3CC4)c3ccccc3n2)c1 ; COc1ccc2c(N(CCC(=O)O)CCc3ccccc3)nc(-c3cccc(Br)c3)nc2c1 ; O=C(O)CCN(CCc1ccccc1)c1nc(-c2cccc(Br)c2)nc2c(F)cccc12]

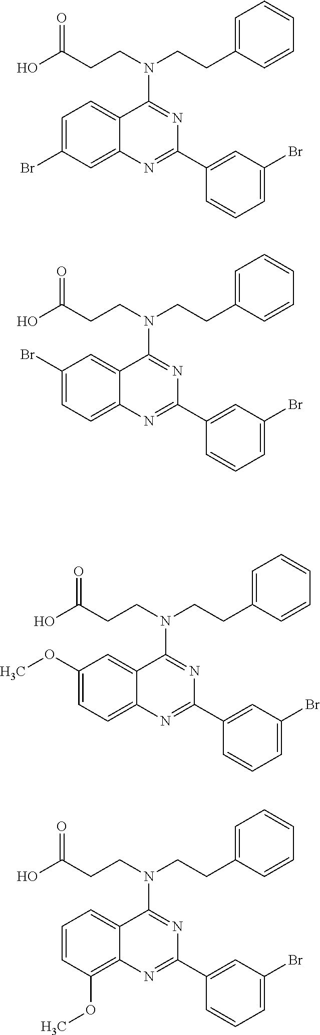

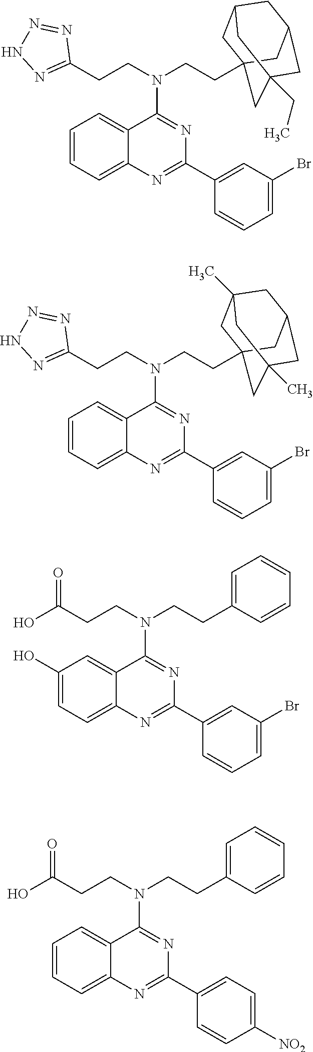

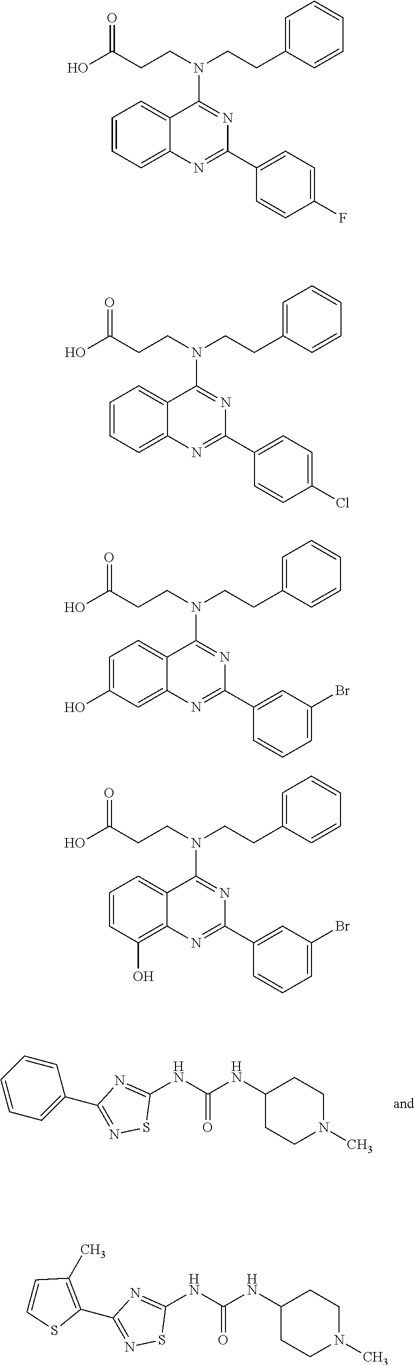

or a pharmaceutically acceptable salt, isomer, solvate, chemically protected form, or prodrug thereof.

In a further particularly preferred embodiment of the first aspect or of the second aspect of the invention, the inhibitor is selected from the following two compounds:

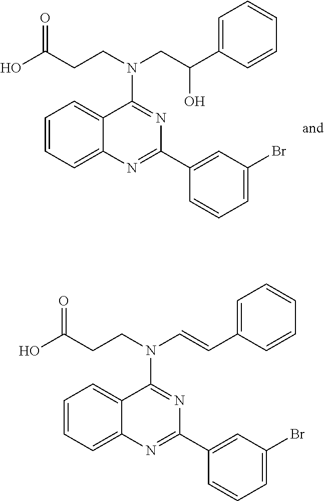 [SMILES: O=C(O)CCN(CC(O)c1ccccc1)c1nc(-c2cccc(Br)c2)nc2ccccc12 ; O=C(O)CCN(/C=C/c1ccccc1)c1nc(-c2cccc(Br)c2)nc2ccccc12]

Without wishing to be bound by a particular theory, the inventors assume that the left one of the above compounds (i.e. COVI-86) will be metabolized in vivo to the right compound (i.e. COVI-87). In a third aspect, the present invention is directed to a pharmaceutical composition comprising the inhibitor according to the first or second aspects of the invention.

In a fourth aspect, the present invention is directed to the inhibitor according to the first or second aspect of the invention or the pharmaceutical composition of the third aspect of the invention for use in therapy.

In a fifth aspect, the present invention is directed to the inhibitor according to the first or second aspect of the invention or the pharmaceutical composition of the third aspect of the invention for use in prophylaxis or therapy of a viral infection.

In a preferred embodiment of the fifth aspect of the invention the viral infection is an infection with a positive stranded single stranded (ss) RNA, preferably of the class of Pisoniviricetes, more preferably a virus of the order Nidovirales, more preferably a virus of the suborder Cornidovirineae and most preferably a virus of the family Coronaviridae. Most preferably the virus is a coronavirus including in particular is SARS-CoV, MERS-CoV, SARS-CoV-2 and mutants thereof.

### EXPERIMENTS

### 1. In Silico Characterization

Initially, a 12 μs molecular dynamics (MD) simulation was run on the first lobe of the SARS-CoV-1 Nsp13 helicase (residues 236 to 440 from the published SARS-CoV-1 Nsp13 PDB: 6JYT)—which shares 100% sequence identity in this specific region with the SARS-CoV-2 ortholog. MD was initiated with implicit solvent. The simulation was run using a step size of 2 femtoseconds. The simulation was run on an NVIDIA Tesla V100-SXM2-32gb GPU. The MD started with an initial minimization step followed by 10,000 steps of equilibration. After which, energies and frames were written every 100 frames. After the MD simulation was complete, every 10^{th} frame was taken from the trajectory. These frames were aligned to the initial pose using and PCA was carried out. Plots of the PCA were generated using the first two principle components. 8 clusters of protein conformations were identified manually. A random point near the center of each cluster was extracted as PDB files. Flexible docking was then preformed into each of the 8 protein conformations on an Intel® Xeon® Platinum 8268 CPU @2.90 GHz cpu. Docking was preformed using a proprietary compound library.

The average docking score from all of the protein conformations was used to rank the ligands from the library. The top 64 ligands were then selected for subsequent biochemical analysis. The in vitro data was correlated the molecular docking into each of the protein frames. The frame with the best correlation with the in vitro FRET-based DNA unwinding assay measurements (FIG. 12) was chosen as the representative structure for use in more rigorous molecular docking for pose refinement of biologically active molecules. Surprisingly, these docking poses revealed a previously unknown allosteric binding site, that is located behind motif II of the active site in the interior of the protein. This pocket is not visible in the crystal structure and could only be identified by the applied molecular dynamics simulations. The pocket shows a tripartite structure with an entry channel, a left channel and a right channel. This pocket is composed of the following amino acids of the SARS-CoV/SARS-CoV-2 Nsp13 amino acids: Y277, T279, L280, Q281, G282, T307, C309, F373, D374, E375, I376, S377, M378, A379, L384, N388, Y396, Y398, I399, G400, D401, P406, P408, N423, V425, C426, R427, and M429. Of these residues, Y277, L384, N388, Y396, Y398, and V425 form the entry channel, T307, C309, F373, D374, E375, S377, M378, A379, and N423 form the left channel and T279, L280, Q281, G282, I376, I399, G400, D401, P406, P408, C426, R427, and M429 form the right channel. Table 1 lists each amino acid directly interacting with the three example compounds, COVI-3, COVI-10, and COVI-35.

TABLE 1

| Residue | COVI-3 | COVI-10 | COVI-35 |
|---|---|---|---|
| Try 277 | X | | |
| Thr 279 | | X | |
| Leu 280 | | X | X |
| Cys 309 | | X | |
| Phe 373 | X | X | X |
| Ile 376 | X | X | X |
| Ala 379 | | X | |
| Asn 381 | X | | |
| Leu 384 | X | X | X |
| Ser 385 | X | | |
| Asn 388 | X | X | |
| Tyr 396 | | X | X |
| Tyr 398 | X | X | X |
| Ile 399 | X | X | X |
| Gly 400 | | X | X |
| Pro 406 | | X | X |
| Pro 408 | | X | X |
| Val 425 | X | X | X |
| Cys 426 | | X | X |
| Met 429 | X | X | X |

FIG. 3 shows an alignment of the amino acid sequences of the seven known helicases of coronoaviruses. The alignment was generated using the Clustal Omega Multiple Sequence Alignment program available at EMBL-EBI (https://www.ebi.ac.uk/Tools/msa/clustalo/) using standard alignment parameters. The amino acids of SARS-CoV-2 Nsp13 that are part of the allosteric binding pocked noted above are highlighted by bold print as are the amino acids of the six other coronavirus helicases that are at the corresponding position in the respective helicase. The following Table 2 summarizes the level of conservation between the amino acids identified as forming the binding pocket of SARS-CoV-2 Nsp13 and the binding pocket of the six other coronavirus helicases.

TABLE 2

| SARS-CoV-2 | | Corresponding AA in | SARS-CoV-2 | | Corresponding AA in |
|---|---|---|---|---|---|
| AA | CONSERVED | other coronaviruses | AA | CONSERVED | other Corona viruses |
| Y277 | NO | Y/I | L384 | YES | L |
| T279 | YES | T | N388 | YES | N |
| L280 | HIGH | L/V/I | Y396 | NO | Y/I |
| Q281 | YES | Q | Y398 | YES | Y |
| G282 | YES | G | I399 | HIGH | I/V |
| T307 | LOW | T/V | G400 | YES | G |
| C309 | LOW | C/A | D401 | YES | D |
| F373 | LOW | F/V | P406 | YES | P |
| D374 | YES | D | P408 | YES | P |
| E375 | YES | E | N423 | YES | N |
| I376 | HIGH | I/V | V425 | HIGH | V/I |
| S377 | YES | S | T426 | NO | C/T |
| M378 | YES | M | R427 | HIGH | R/K/Q |
| A379 | NO | A/C/L | M429 | YES | M |

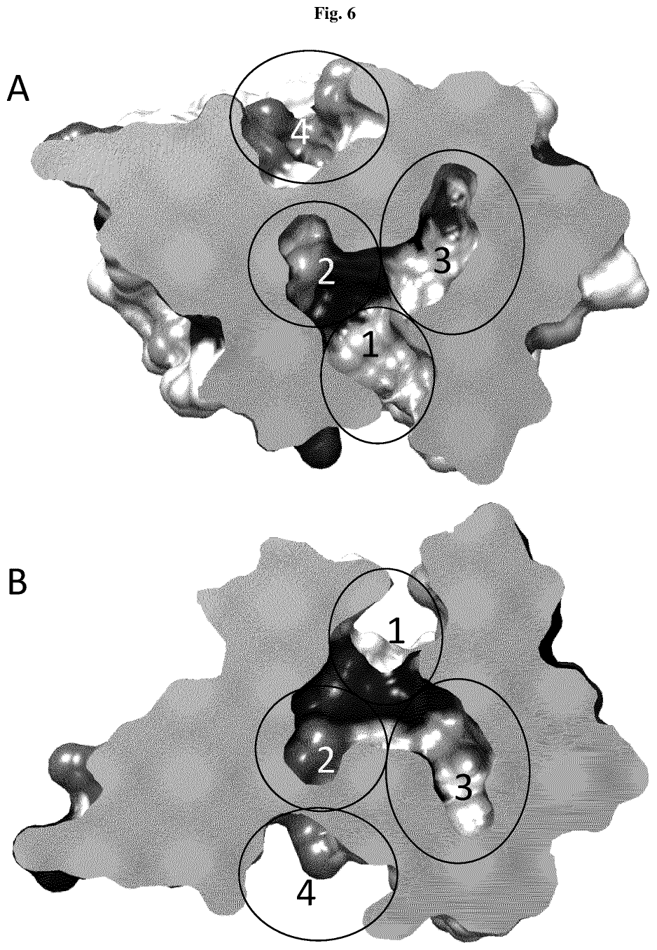

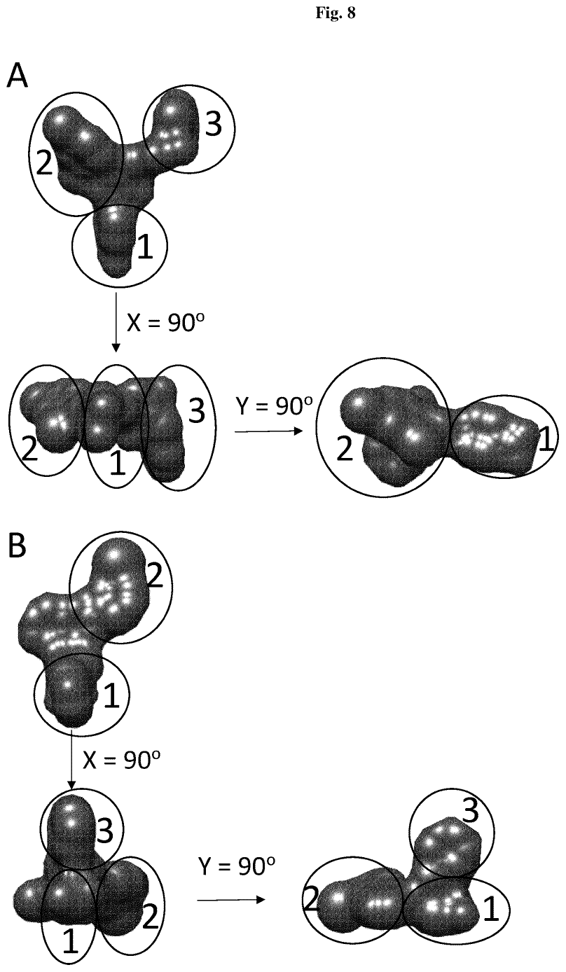

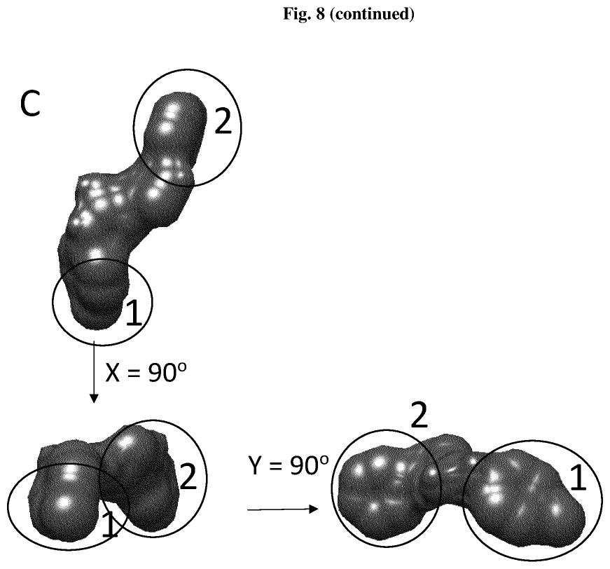

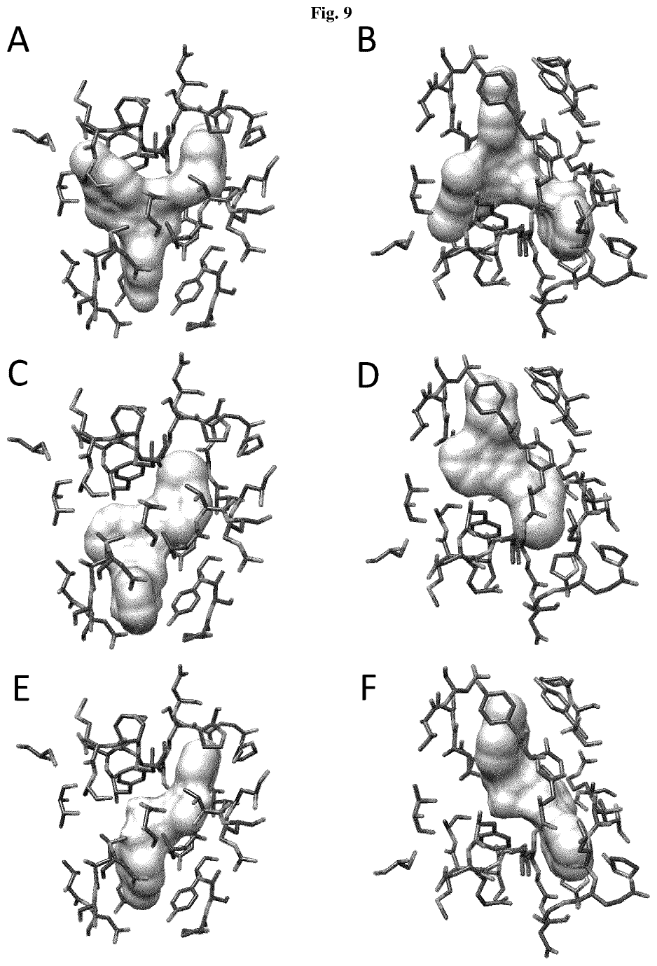

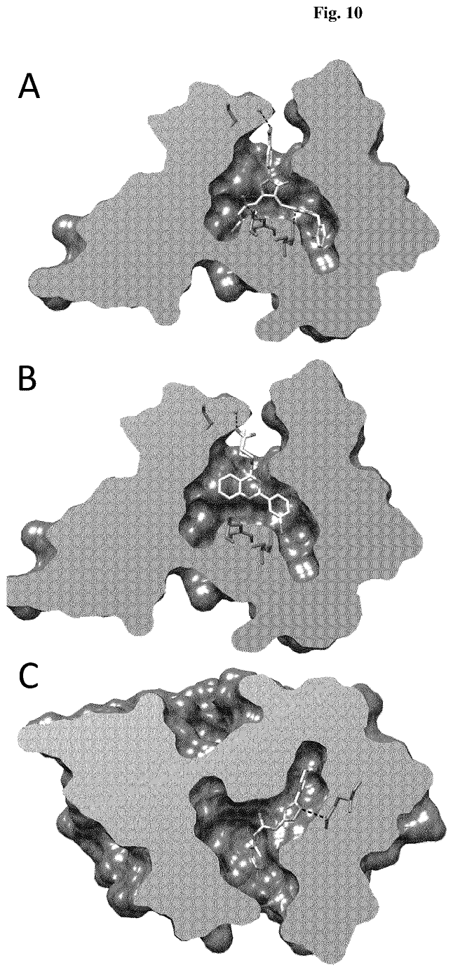

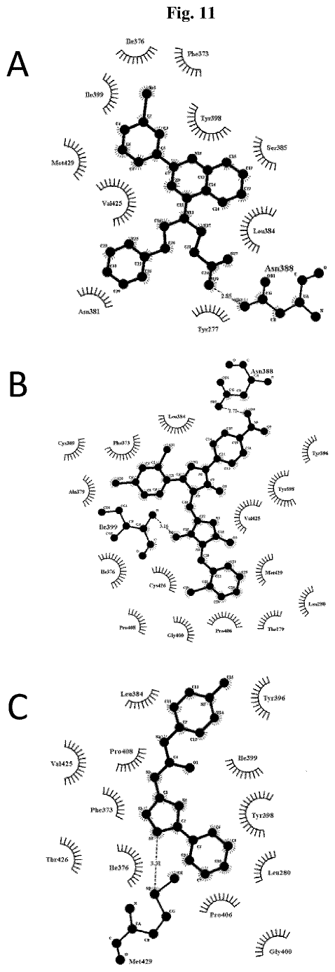

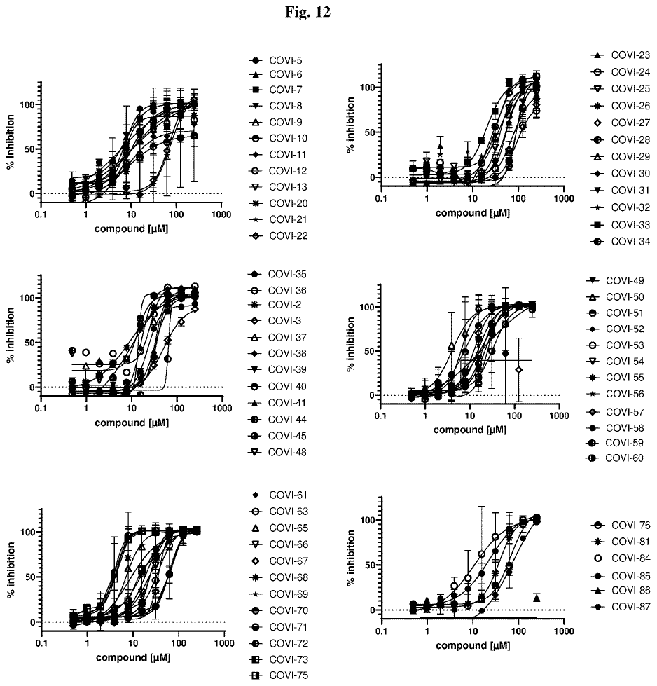

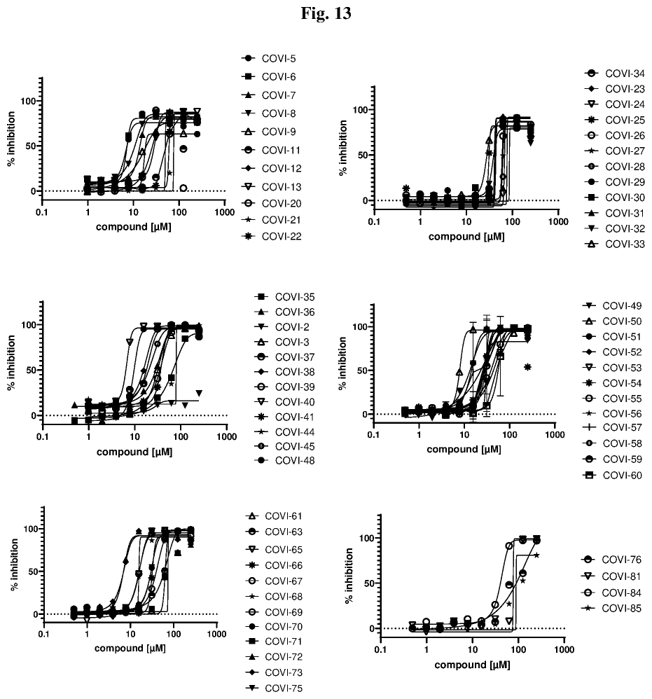

For the development of additional small molecule inhibitors that both fit into the binding pocket and have specific, favorable interactions, special attention has been paid to the following interactions between potential small molecules and the protein. Although the binding pocket is largely hydrophobic and thus binding will be dominated by hydrophobic interactions, the number of favorable interactions below should be maximized while maintaining physicochemical properties conducive to favorable pharmacokinetic properties.
(i) Hydrogen Bonds: Hydrogen bonding (H-bonding) is likely the most critical aspect for small molecule binding in general. H-bonds are formed between lone pairs of electrons and polar, electron poor hydrogens at distances of 1.5 Å to 2.5 Å. The backbone of every amino acid contains both an H bond donor and acceptor, and many amino acid sidechains contain H-bond donors, acceptors, or both. The right and left channels of the binding pocket are rich in both H-bond donors and acceptors. Fewer H-bond donors and acceptors are present in the entry channel, especially as the channel nears the center of the protein. This can be seen in FIG. 6 where black regions are hydrophobic, and thus poor in H-bond accepting and donating groups. To take advantage of this, H-bond acceptors and donators should be positioned on small molecule moieties directed into the left and right channel, as well as the entry channel outer boundary.
(ii) Salt Bridges: Since several residues lining the binding pocket have charged sidechains—specifically aspartate 374 and 401, glutamate 375, and to some extent Arginine 427—the potential to form salt bridges between charged amino acid sidechains and charged moieties of the small molecule. Salt bridges are ideally formed at a distance of approximately 2 Å and can have interaction energies in the low double digit kcal/mol, but often suffer from the large desolvation penalty associated with the charged moiety of the small molecule. To take advantage of salt bridges, charged moieties may be directed toward the left and right channels.
(iii) pi-pi stacking: Interactions between pi bonded systems, especially aromatic pi bonded systems, are among the most prevalent intra- and intermolecular interactions. In proteins, several amino acids are capable of pi stacking with aromatic moieties in small molecules, these are phenylalanine, tyrosine, tryptophan, and histidine. In the case of the SARS-CoV-2 Nsp13 helicase allosteric binding pocket, one phenylalanine is present for pi stacking in the left channel, two tyrosines are present in the entry channel, and one tyrosine is shared between the entry and right channel. These aromatic systems can be targeted for pi stacking by including complementary aromatic moieties in small molecules. Both face-to-face and edge-to-face interactions are highly favorable at distances of 3.5 Å-5 Å.
(iv) Cation/Polar-pi: Cation-pi and polar-pi interactions occur between an aromatic pi system and a cation or electron poor polar region. These interactions, as in (iii) are capable of being exploited in the left and entry channels of the binding pocket with phenylalanine and tyrosine. To target these residues, the small molecules should contain polar regions or cations addressing the entry and left channels primarily. Furthermore, pi systems in the small molecules may interact with polar or charged amino acid sidechains, which are primarily present at the entry channel outer boundary, left, and right channels.
(v) Halogen bonding: Carbo halogens in small molecules have three critical electron interactions. The first is with the backbone carbonyl oxygen, which tend to occur at distances from 2.5 Å to 3.5 Å and become more favorable as the Van der Waals radius of the halogen increases. Additionally, carbo halogens interact with pi systems in a similar manner to the polar-pi interaction described in (iv). Finally, carbo halogens, especially fluorine, can form tight bonds with sulfur atoms, especially those in cystines. Halogens can be added in many locations on the small molecule, since there are potential binding interactions in many places in the binding pocket. There are pi systems able to be targeted by halogens in the left, right, and entry channel. There are backbone carbonyl oxygens able to be targeted primarily in the left and right channels. Cystine sulfurs are available in the left and right channel.
(vi) Van der Waals: nonspecific interactions between uncharged, nonpolar atoms of the protein and small molecule are the most abundant present as well as the weakest. They are present in all channels within the binding pocket but are the only available interactions in the center of the entry channel, as can be observed in FIG. 8 by the concentration of hydrophobic residues in this region. As such, in the development of new compounds, nonpolar substituents should be directed toward the center of the entry pocket.

The structure of the compounds subsequently tested in below assays were determined using this approach and then synthesized by various contract manufacturers.

### 2. Nsp13 Expression and Purification

A plasmid coding for the SARS-CoV-2 Nsp13 helicase with an N-terminal His10-SUMO-tag was co-transformed with pGro7 (Takara Chaperone plasmid set #3340) into E. coli BL21 Gold (DE3) expression strain (Agilent Technologies 230132), plated on an LB-Agar plate supplemented with Chloramphenicol and Kanamycin and grown at 37° C. overnight. A swab from the LB-Agar plate was used to inoculate the pre-culture (LB supplemented with Chloramphenicol and Kanamycin) and grown over night at 30° C. while shaking. The pre-culture was used the next day to inoculate the expression culture 1:50. The expression culture was cultivated by shaking at 37° C. until an optical density (600 nm) of 0.5-0.8 was reached. The chaperone expression was then induced by the addition of 0.5 mg/ml (final concentration) L-arabinose. After additional shaking at 37° C. for 1 h, the cultures were transferred to 25° C. and the expression of Nsp13 was induced by the addition of 0.5 mM IPTG (final concentration). The expression was allowed to proceed for 15 h at 25°, before the bacterial cells were harvested by centrifugation.

The cells were resuspended in 50 mM Tris pH 7.5, 500 mM NaCl, 1 mM MgCl₂, 20 mM Imidazole, 1 mM DTT supplemented with EDTA-free protease inhibitors (Roche) and lysed by sonication. The lysate was spun down for 30 min at 20 000×g (4° C.). The protein was purified by IMAC using lysis buffer as wash buffer and 50 mM Tris pH 7.5, 500 mM NaCl, 1 mM MgCl₂, 500 mM Imidazole, 1 mM DTT as elution buffer. Elution fractions containing the target protein were pooled, supplemented with 1 mg His6-SenP2 protease and dialyzed extensively against 20 mM Tris pH 7.5, 300 mM NaCl, 1 mM MgCl2, 1 mM DTT. The dialyzed protein solution was spiked with 5 M NaCl to 500 mM final concentration. Uncleaved His10-SUMO-Nsp13 and His6-SenP2 were removed by IMAC. The flow through containing the untagged target protein was diluted with 20 mM Tris pH 7.5 to a final NaCl concentration of 50 mM and loaded on a Heparin column operated in 20 mM Hepes pH 7.5, 50 mM NaCl, 1 mM DTT, and eluted with a gradient to 50% 20 mM Hepes pH 7.5, 1 M NaCl, 1 mM DTT. The pure peak fractions were pooled, supplemented with 20% (v/v) glycerol and flash frozen in liquid nitrogen for storage at −80° C.

### 3. Nsp13 Helicase Unwinding Assay

A FRET-based DNA unwinding assay was established for SARSCoV2 Nsp13 with an amino acid sequence according to SEQ ID NO: The helicase unwinding assay monitors fluorescence resonance energy transfer (FRET) to detect the separation of a fluorophore-labeled reporter strand from a loading strand that is modified with a spectrally paired quencher dye by Nsp13. Helicase unwinding reactions were performed in 384 well plates in 10 mM HEPES pH 7.4, 10 mM NaCl, 0.005% BSA, 2.5% Glycerol, 2.5 mM MgCl₂, 0.01% CHAPS, 2 mM DTT and reaction mixtures contained 0.15 nM Nsp13 and 100 nM of fluorescein- and black hole quencher labeled dsDNA. The unwinding reaction was initiated by the addition of 200 μM ATP and 1 μM of unlabeled single-stranded DNA (ssDNA/capture oligo which prevents re-annealing and will permanently separate the reporter strand from the loading strand) followed by shaking the plates for 5 see at 1450 rpm and sealing them with a foil compatible with fluorescence reading. The change in fluorescence was immediately recorded with a BMG labtech PheraStar FSX reader (excitation wavelength 485 nm, emission wavelength 520 nm). Unless stated otherwise, unwinding proceeded for 20 min. The increase in fluorescence was plotted as a function over time and the initial velocities of Nsp13-mediated unwinding were obtained by fitting the resulting kinetic trace by a linear curve fit. To profile compounds that modulate the unwinding activity of Nsp13, the enzyme and substrate were incubated for 30 min in the presence of the compound prior to initiating unwinding by ATP addition. The rate of unwinding (initial velocity) was determined as described above and compared against the rate of unwinding in the absence of the putative modulator/compound.

### 4. Malachite Green ATPase Assay

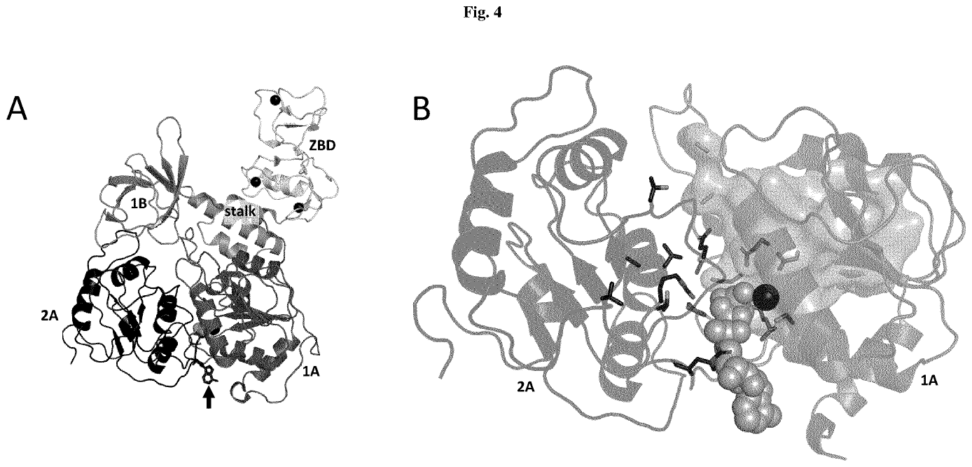

To confirm active compounds by an orthogonal method, a malachite green assay was established to validate the small molecules as inhibitors of the Nsp13 enzymatic ATP hydrolysis (FIG. 4). The malachite green assay allows the detection of organic phosphate that is released upon ATP hydrolysis by the helicase. The 10 μL ATPase assay reactions were performed in transparent 384 well plates in 10 mM HEPES pH 7.4, 10 mM NaCl, 0.005% BSA, 2.5% Glycerol, 2.5 mM MgCl₂, 0.01% CHAPS, 2 mM DTT and 2.5% DMSO. The reaction mixtures contained 500 μM Nsp13 and 100 nM dsDNA of the same sequence as for the helicase assay. The reaction was started by the addition of 100 μM ATP followed by shaking the plates for 5 sec at 1450 rpm. The reaction was allowed to proceed for 5 minutes, before 20 μL Biomol Green (Enzo Life Sciences) was added to stop the reaction. The absorbance of the phosphomolybdate-malachite green complex was monitored at 650 nm on a Tecan Genios pro 4 minutes after stopping the reaction. Compounds inhibiting the ATPase activity of SARS-CoV-2 Nsp13 were profiled by incubating the compounds for 30 min with the enzyme substrate complex prior to starting the reaction with ATP. The inhibition was calculated by normalizing the blank-subtracted absorbance reads of to untreated blank-subtracted controls.

### 5. SARS-CoV-2 Nanoluciferase Assay in Human ACE-2 Expressing A549 Cells

The SARS-CoV-2 nanoluciferase assay using A549 cells expressing the human ACE-2 receptor was performed in the laboratory of Pei-Yong Shi at the University of Texas, Medical Branch on behalf of Eisbach. A549-ACE2 cells (12,000 cells per well) were seeded in phenol-red free medium supplemented with 2% FBS into clear 96-well plates. On the next day, 2-fold serial dilutions of compounds were added 1 h prior to infection with SARS-CoV-2-Nluc. At 48 h post-infection, luciferase signals were measured in lysed cells using Nano luciferase substrates (Promega). The relative luciferase signals were calculated by normalizing the luciferase signals of the compound-treated groups to that of the DMSO-treated groups (set as 100%). Experiments were performed at one (MOI=0.025) with technical duplicates.

6. SARS-CoV-2 Antiviral Assay with Patient Isolate in VeroE6 Cells

### TCID50 Readout

VeroE6 (10,000 cells per well) were seeded in DMEM medium supplemented with 10% FBS and 100 U penicillin/0.1 mg/ml streptomycin in a clear 96 well plate. On the next day, 2-fold serial dilutions of compounds were added 1 h prior to infection with SARS-CoV-2 (primary SARS-CoV-2 isolate, Dusseldorf strain, MOI=1). 1 h after infection, cells were washed thrice with PBS and compounds were replenished. The cell culture medium from each well was collected 24 hr after infection and subjected to TCID50 determination. TCID50/mL values were determined by crystal violet staining and subsequent scoring of the amounts of wells displaying cytopathic effects. TCID50 was calculated by the Spearman-Karber algorithm.

### CPE-Based Assay

VeroE6 (10,000 cells per well) were seeded in DMEM medium supplemented with 10% FBS and 100 U penicillin/0.1 mg/ml streptomycin in a clear 96 well plate. On the next day, 2-fold serial dilutions of compounds were added 1 h prior to infection with SARS-CoV-2 (primary SARS-CoV-2 isolate, Dusseldorf strain). Cell viability was determined by SRB assay 72 h post infection. Absorbance at 510 nm was determined in a Tecan plate reader. Experiments were performed at one MOI (MOI=0.01, 2000 TCID50) with technical quadruplicates. Wells containing cells only and cells infected with virus were used as internal controls. Percentage of cytopathic effect (CPE) inhibition was defined as [(test compound−virus control)/(cell control−virus control)]*100.

### N-Protein ELISA

VeroE6 (1,000 cells per well) were seeded in DMEM medium supplemented with 10% FBS and 100 U penicillin/0.1 mg/ml streptomycin in a clear 96 well plate. On the next day, 2-fold serial dilutions of compounds were added 1 h prior to infection with SARS-CoV-2 (primary SARS-CoV-2 isolate, Dusseldorf strain). After another 24 hours cells were fixed with 4% PFA and permeabilized with Triton and blocking is done with FCS. The primary antibody is directed against the N-protein. TMB was used as substrate. The reaction was stopped after 15 minutes by addition of HCl and then measured at 450 nm in the ELISA reader. The measurements were normalized to cells treated only with live virus and DMSO.

### 7. HCoV-229e-Rluc Antiviral Assay

Huh7 cells (100,000 cells per well) were seeded in DMEM medium supplemented with 10% FBS and 100 U penicillin/0.1 mg/ml streptomycin into white 96-well plates. On the next day, 2-fold serial dilutions of compounds were added 1 h prior to infection with hCoV-229e-Rluc. At 24 h post-infection, luciferase signals were measured in lysed cells using Coelenterazine as bioluminescent substrate and a luminometer for readout. The relative luciferase signals were calculated by normalizing the luciferase signals of the compound-treated groups to that of the DMSO-treated groups (set as 100%). Experiments were performed at an MOI=0.01 with technical duplicates.

### 8. Synthesis of Chemical Compounds

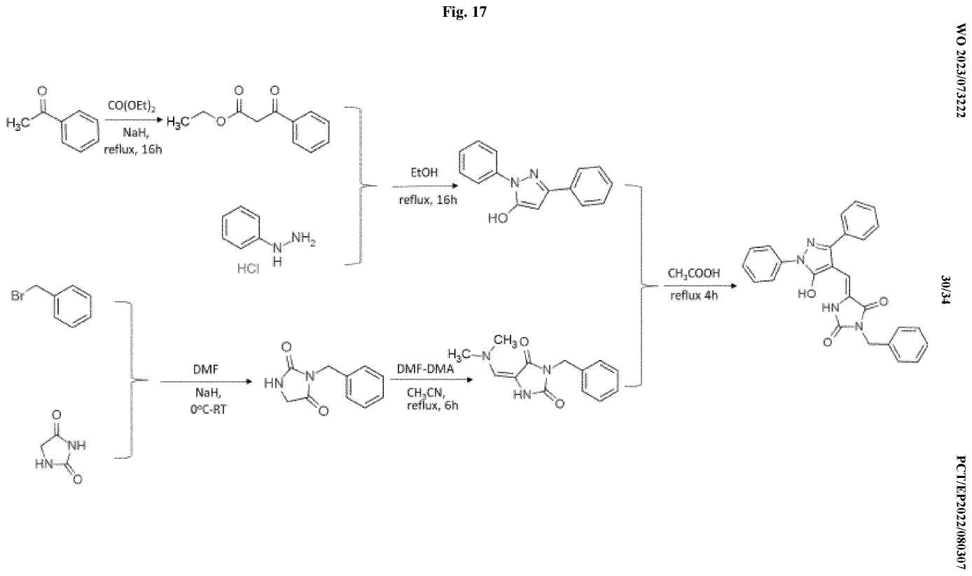

The synthesis of compounds of general formula (I) can be carried out according to the general synthesis scheme shown in FIG. 17. The compound prepared in FIG. 17 is not shown in the list of compounds depicted in FIG. 14 but rather reflects a basic compound scaffold in which the phenyl groups are not substituted (while several compounds shown in FIG. 14 carry substituents at the phenyl groups).

For example, the synthesis of COVI-06 consists of first an N-alkylation of hydantoin with 3-fluorobenzyl bromide in DMF and NaH as a base at 0° C. and warming up to room temperature. In step 2, the product of step 1, 3-(3-fluorobenzyl)-2,4-imidazolidinedione, undergoes a condensation reaction with DMF under reflux conditions for 6 h to form (5Z)-5-[(dimethylamino)methylidene]-3-[(3-methylphenyl)methyl]imidazolidine-2,4-dione. Step 3 consists of an aldol reaction of 1-(4-chlorophenyl)ethan-1-one and dimethyl carbonate under reflux conditions for 16 hours, forming ethyl 3-(4-chlorophenyl)-3-oxopropanoate. Next, ethyl 3-(4-chlorophenyl)-3-oxopropanoate and (4-nitrophenyl)hydrazine hydrochloride form pyrazole 3 by refluxing the reaction mixture in ethanol for 16 hour. The product of this step 4 is 3-(4-chlorophenyl)-1-(4-nitrophenyl)-1H-pyrazol-5-ol, which is subsequently reacted with (5Z)-5-[(dimethylamino)methylidene]-3-[(3-methylphenyl)methyl]imidazolidine-2,4-dione, the product from step 2 under reflux conditions for 4 hours in the presence of acetic acid to form COVI-06.

The synthesis of COVI-20 consists first of a conversion of (3-methoxyphenyl)methanol to form 1-(bromomethyl)-3-methoxybenzene. As expected, the subsequent steps mirror those for the synthesis of COVI-06. Step 2 is an N alkylation reaction between 1-(bromomethyl)-3-methoxybenzene and hydantoin with DMF and NaH from 0° C. to room temperature resulting in the formation of 2-hydroxy-3-[(3-methoxyphenyl)methyl]imidazolidin-4-one. In step 3, 2-hydroxy-3-[(3-methoxyphenyl)methyl]imidazolidin-4-one undergoes a condensation reaction with DMF under reflux conditions for 6 hours to form (5Z)-5-[(dimethylamino)methylidene]-2-hydroxy-3-[(3-methoxyphenyl)methyl]imidazolidin-4-one. Step 4 consists of an aldol reaction of 1-(4-chlorophenyl)ethan-1-one and dimethyl carbonate under reflux conditions for 16 hours, producing ethyl 3-(4-chlorophenyl)-3-oxopropanoate. Next, in step 5, ethyl 3-(4-chlorophenyl)-3-oxopropanoate and (4-nitrophenyl)hydrazine hydrochloride are combined in a pyrazole formation reaction in ethanol under reflux conditions for 16 hour. The product of this step 4 is 3-(4-chlorophenyl)-1-(4-nitrophenyl)-1H-pyrazol-5-ol, which is subsequently reacted with (5Z)-5-[(dimethylamino)methylidene]-2-hydroxy-3-[(3-methoxyphenyl)methyl]imidazolidin-4-one in the presence of acetic acid under reflux conditions for 4 hours to form COVI-20.

Likewise, the synthesis of compounds of general formula (II) can be carried out according to the general synthesis scheme shown in FIG. 18. Again, the compound prepared in FIG. 18 is not shown in the list of compounds depicted in FIG. 14 but rather reflects a basic compound scaffold in which the phenyl groups are not substituted (while several compounds shown in FIG. 14 carry substituents at the phenyl groups).

Specifically, the synthesis of COVI-3 begins with a quinolinone formation between 2-aminobenzamide and 3-bromobenzaldehyde in step 1. In step 2, the product of step 1, 2-(3-bromophenyl)quinazolin-4-ol undergoes chlorination by phosphoryl chloride to form 2-(3-bromophenyl)-4-chloroquinazoline. 2-(3-bromophenyl)-4-chloroquinazoline then undergoes an amination reaction in step 3 with ethyl 3-[(2-phenylethyl)amino]propanoate—which is produced via an alkylation reaction between ethyl 3-aminopropanoate and (2-bromoethyl)benzene in the presence of DMF and potassium carbonate at 120° C. for 2 hours—again in the presence of DMF and potassium carbonate at 120° C. for 8 hours in order to form ethyl 3-{[2-(3-bromophenyl)quinazolin-4-yl](2-phenylethyl)amino}propanoate in step 4. Ethyl 3-{[2-(3-bromophenyl)quinazolin-4-yl](2-phenylethyl)amino}propanoate is subsequently subjected to an ester hydrolysis reaction with sodium hydroxide and THF—H₂O from 0° C. to room temperature for 16 hours followed by reflux for 3 hours to form COVI-3.

Similarly, COVI-72 synthesis begins with a quinolinone formation between 2-aminobenzamide and 3-bromobenzaldehyde in step 1. In step 2, the product of step 1, 2-(3-bromophenyl)quinazolin-4-ol undergoes chlorination by phosphoryl chloride and forms 2-(3-bromophenyl)-4-chloroquinazoline. In parallel 2-(3-ethyladamantan-1-yl)acetic acid undergoes amide formation via acid chloride to form, 2-(3-ethyladamantan-1-yl)acetamide, which is reduced to the corresponding amine with lithium aluminum hydride and THF for 2 hours to form 2-(3-ethyladamantan-1-yl)ethan-1-amine. Next, in step 3, 2-(3-ethyladamantan-1-yl)ethan-1-amine undergoes cyanoalkylation with prop-2-enenitrile through heating at 100° C. for 1 hour. The product of step 3, 3-{[2-(3-ethyladamantan-1-yl)ethyl]amino}propanenitrile, is employed for aminoquinolinone formation with 2-(3-bromophenyl)-4-chloroquinazoline with DIPEA as base and dioxane at 120° C. for 16 hours, yielding 3-{[2-(3-bromophenyl)quinazolin-4-yl][2-(3-ethyladamantan-1-yl)ethyl]amino}propanenitrile.

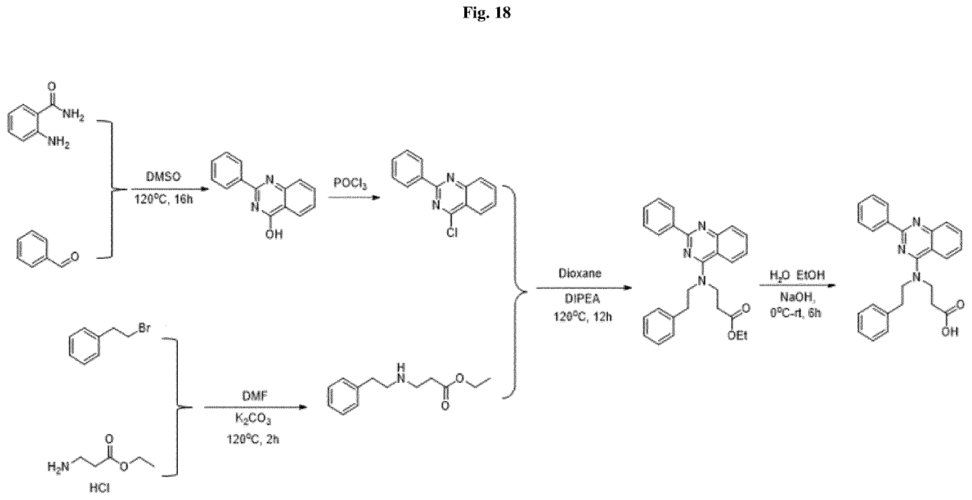

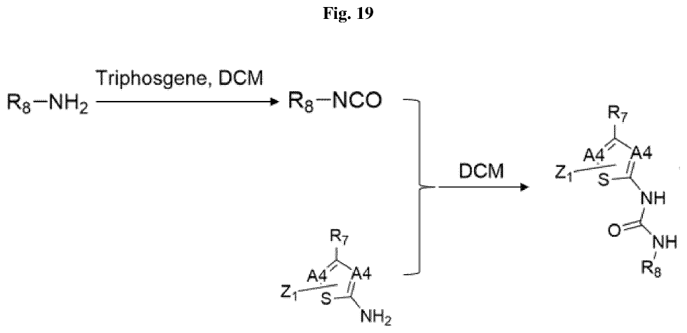

Finally, in step 5 tetrazole formation, 3-{[2-(3-bromophenyl)quinazolin-4-yl][2-(3-ethyladamantan-1-yl)ethyl]amino}propanenitrile is converted to COVI-72 heating L-proline, DMF, and sodium azide at 120° C. for 24 hours. Likewise, the synthesis of compounds of general formula (III) can be carried out according to the general synthesis scheme shown in FIG. 19.

Specifically, COVI-35 can be produced in two steps. First, an aminothiazole formation is carried out using 2-bromo-1-phenylethan-1-one and thiourea in ethanol at 50° C. for 6 hours, producing 4-phenyl-1,3-thiazol-2-amine. 4-phenyl-1,3-thiazol-2-amine then takes part in urea formation reaction with 1-methylpiperidin-4-amine, triphosgene and DCM for 2 hours to form COVI-35.

Additionally, COVI-85 may be produced in three steps, beginning with a Pinner reaction between ethanol and 3-methylthiophene-2-carbonitrile, catalyzed by HCl to form ethyl 3-methylthiophene-2-carboximidate. Similar to COVI-35, an aminothiadiazole formation follows between ethyl 3-methylthiophene-2-carboximidate and thiourea with potassium tert-butoxide, DMSO, and molecular iodine for 16 hours at room temperature. This forms 3-(3-methylthiophen-2-yl)-1,2,4-thiadiazol-5-amine, which is used in the step 3 urea formation reaction with 1-methylpiperidin-4-amine, triphosgene and DCM for 2 hours to form COVI-85.

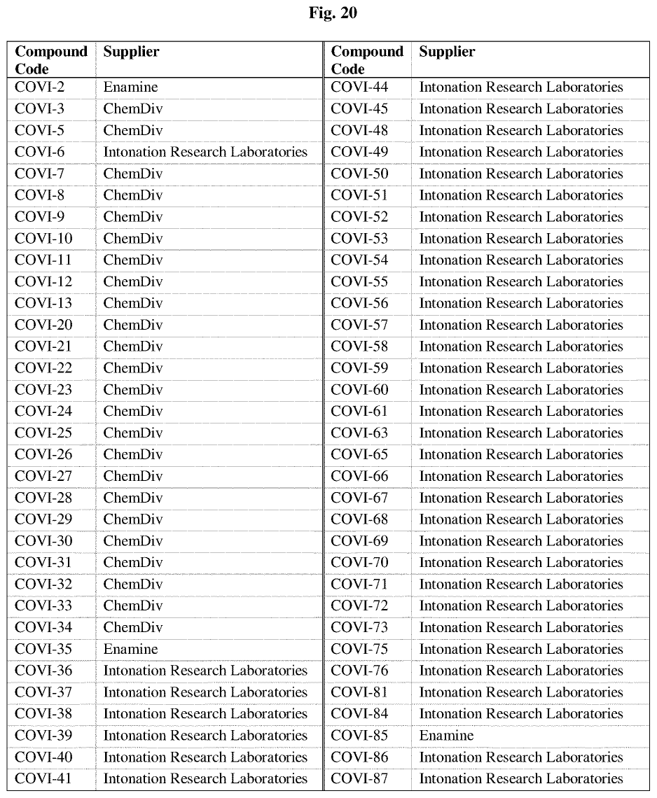

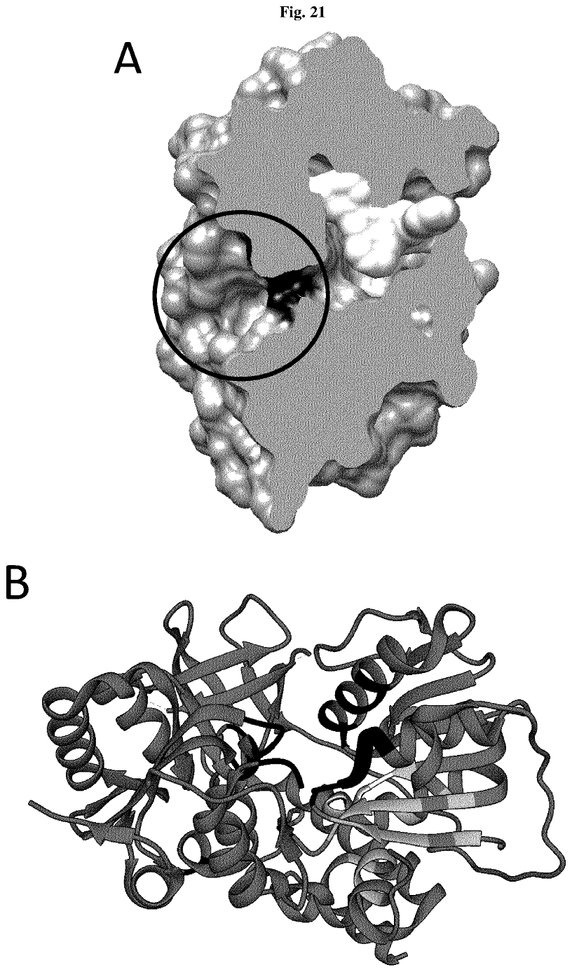

All compounds that have been rationally designed as described in Example 1 were ordered from the manufacturers Enamine (Riga, Latvia), ChemDiv (San Diego CA, USA), or Intonation Research Laboratories (Hyderabad, India) under CDA as shown in the table in FIG. 20. The inhibitors of the invention are simple molecules to synthesize and it is within the ability of the skilled person to synthesize such molecules without any inventive activity. Accordingly, there is a large number of contract manufacturers that can produce such compounds to order once provided with the structure to synthesize.

1. An inhibitor of the helicase activity of a Nsp13 helicase of SARS-CoV-2 (Nsp13) or of a viral homologue thereof, wherein the inhibitor specifically binds to an allosteric binding pocket within the N-terminal lobe of the ATPase domain of Nsp13 or of a viral homologue thereof.

2. The inhibitor of claim 1, with a structure according to formula (I): 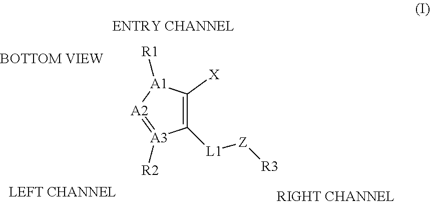
wherein
A1 and A3 are each independently selected from N or C;
A2 is selected from N, C or 0;
X is H or OH or NH₂;
L1 is selected from the group consisting of C, CH, CH₂, O, N, and NH; or L1 is not present;
Z is any 5-, 6- or 7-membered carbo- or heterocycle substituted with R3, optionally further substituted, wherein R3 is connected to Z either via a bond or via a —CH₂— group;
R1 is H or any 5-, 6- or 7-membered carbo- or heterocycle, optionally substituted with one, two or three substituents that are independently from each other selected from the group consisting of —NO₂, —CN, —OH, —COOH, —NH—SO₂-alkyl, NH—CO-alkyl, —CONH₂, —CONH-alkyl, —Br, —Cl, —F, —I, Me, —CF₃, Et, —OMe, and —SMe;
R2 is H or any 5-, 6- or 7-membered carbo- or heterocycle, optionally substituted with one, two or three lipophilic substituents that are independently from each other selected from the group consisting of —Br, —Cl, —F, —I, Me, —CF₃, Et, —OMe, and —SMe, wherein R2 is connected to A3 either via a bond or via a —CH₂— group;
R3 is H or any 5-, 6- or 7-membered carbo- or heterocycle, optionally substituted with one, two or three substituents that are independently from each other selected from the group consisting of —NO₂, —CN, —OH, —COOH, —NH—SO₂-alkyl, NH—CO-alkyl, —CONH₂, —CONH-alkyl, —Br, —Cl, —F, —I, Me, —CF₃, Et, —OMe, and —SMe;
or a pharmaceutically acceptable salt, isomer, solvate, chemically protected form, or prodrug thereof.

3. The inhibitor of claim 2, with a structure according to formula (I), wherein
A1 is N substituted with R1;
A2 is N;
A3 is C substituted with R2, wherein R2 is connected to A3 either via a bond or via a —CH₂— group;
X is H or OH;
L1 is CH═ or CH₂ connected to Z;
Z is a 5- or 6-membered heterocycle substituted with R3, optionally further substituted, wherein R3 is connected to Z either via a bond or via a —CH₂— group;
R1 is a 6-membered aryl or a 5- or 6-membered heteroaryl, optionally substituted with one, two or three substituents that are independently from each other selected from the group consisting of —NO₂, —CN, —OH, —COOH, —NH—SO₂-alkyl, NH—CO-alkyl, —CONH₂, —CONH-alkyl, —Br, —Cl, —F, —I, Me, —CF₃, Et, —OMe, and —SMe;
R2 is 6-membered aryl or a 5- or 6-membered heteroaryl, optionally substituted with one, two or three lipophilic substituents that are independently from each other selected from the group consisting of —Br, —Cl, —F, —I, Me, —CF₃, Et, —OMe, and —SMe; and
R3 is 6-membered aryl or a 5- or 6-membered heteroaryl, optionally substituted with one, two or three substituents that are independently from each other selected from the group consisting of —NO₂, —CN, —OH, —COOH, —NH—SO₂-alkyl, NH—CO-alkyl, —CONH₂, —CONH-alkyl, —Br, —Cl, —F, —I, Me, —CF₃, Et, —OMe, and —SMe.

4. The inhibitor of claim 2, with a structure according to formula (I), wherein
A1 is N substituted with R1;
A2 is N;
A3 is C substituted with R2, wherein R2 is connected to A3 either via a bond or via a —CH₂— group;
X is H or OH;
L1 is CH═ connected to Z;
Z is a 5-membered heterocycle substituted with R3, optionally further substituted, wherein R3 is connected to Z either via a bond or via a —CH₂— group;
R1 is 6-membered aryl or a 5- or 6-membered heteroaryl, optionally substituted with one, two or three substituent(s) that are independently from each other selected from the group consisting of —NO₂, —CN, —OH, COOH, NH—SO₂-alkyl, NH—CO-alkyl, —CONH₂, —CONH-alkyl, —Br, —Cl, —F, —I, Me, —CF₃, Et, —OMe, and —SMe;
R2 is 6-membered aryl or a 5- or 6-membered heteroaryl, optionally substituted with one, two or three lipophilic substituent(s) selected from the group consisting of —Br, —Cl, —F, —I, Me, —CF₃, Et, —OMe, and —SMe,
R3 is 6-membered aryl or a 5- or 6-membered heteroaryl, optionally substituted with one, two or three substituent(s) that are independently from each other selected from the group consisting of —NO₂, —CN, —OH, —COOH, —NH—SO₂-alkyl, NH—CO-alkyl, —CONH₂, —CONH-alkyl, —Br, —Cl, —F, —I, Me, —CF₃, Et, —OMe, and —SMe.

5. The inhibitor of claim 2, with a structure according to formula (I), wherein
R1 is phenyl, 4-nitrophenyl, 2,6-dichloro-4-bromo-phenyl, 4-fluorophenyl, 4-bromophenyl, 2,6-dichlorophenyl, 2-methylphenyl, 4-methylphenyl, 2,3-dimethylphenyl, or 4-methoxyphenyl;
R2 is 2,4-dichlorophenyl, 4-chlorophenyl, phenyl, benzyl, 3-pyridine, 2-methylbenzyl, 4-fluorobenzyl, 4-chlorobenzyl, 2-methoxyphenyl, or 3-methoxyphenyl; and
R3 is 4-fluorophenyl, 2-fluorophenyl, 3-fluorophenyl, 2,4-difluorophenyl, 2-methylphenyl, 3-methylphenyl, 4-methylphenyl, 2-chlorophenyl, 3-chlorophenyl, 3-pyridine, 3-methoxyphenyl, 4-nitrophenyl, 3-nitrophenyl, 4-trifluoromethyl-phenyl, or 4-cyanophenyl.

6. The inhibitor of claim 1, with a structure according to formula (II): 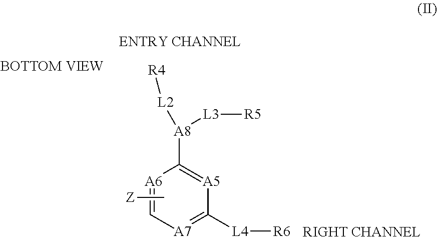
wherein
A5 and A8 are each independently selected from N or CH;
A6 is selected from N or CH, or when A6 takes part in the annulated carbo- or heterocycle Z, then A6 is C;
A7 is selected from N or CH, or when A7 takes part in the annulated carbo- or heterocycle Z, then A7 is C;
L2 is selected from the group consisting of —CH₂—R4, —CF₂—R4, —CH₂—CH₂—R4, —CH₂—CH₂—CH₂—R4, —O—R4, —NH—R4, —N═R4;
L3 is selected from the group consisting of CH₂—R5, —CF₂—R5, —CH₂—CH₂—R5, —CH₂—COH₂—R5, —CH═CH—R5, —CH₂—CF₂—R5, —CH₂—CH₂—CH₂—R5, —O—R5, —NH—R5, —N═R5;
or
L2 and L3 together with the A8 to which they are connected form a 5- or 6-membered heterocycle substituted by R4 and/or R5;
L4 is CH₂, —CF₂—, CH₂—CH₂, CH₂—CH₂—CH₂, O, N, and NH, or is absent;
Z is a 5-, 6- or 7-membered carbo- or heterocycle, optionally substituted with one, two, or three substituent(s) selected from the group consisting of —Br, —Cl, —F, —I, —OH, Me, —CF₃, Et, —OMe, —SMe, and —NO₂;
and can be annulated to the central core or connected via a covalent bond R4 is 5-, 6- or 7-membered carbo- or heterocycle, optionally substituted with one, two, or three lipophilic substituent(s) selected from the group consisting of —Br, —Cl, —F, —I, —CF₃, Me, Et, —OMe, and —SMe, or R4 is hydrogen, methyl, or COOH;
R5 is a 5-, 6-, 7-, 8-, 9- or 10-membered carbo- or heterocycle, optionally substituted with one, two, or three lipophilic substituents selected from the group consisting of —Br, —Cl, —F, —I, Me, —CF₃, Et, —OMe, and —SMe;
R6 is a 5-, 6- or 7-membered carbo- or heterocycle, optionally substituted with one, two, or three substituents selected from the group consisting of —Br, —Cl, —F, —I, —OH, —NO₂, Me, —CF₃, Et, —OMe, and —SMe;
or R6 is H;
or
when A7 takes part in the annulated carbo- or heterocycle Z then A5 and A6 are independently selected from —N or —CH and A8 is selected from —N, —CH, —CH₂—N, —CH₂—CH, or —NH—CH
or a pharmaceutically acceptable salt, isomer, solvate, chemically protected form, or prodrug thereof.

7. The inhibitor of claim 6, with a structure according to formula (II), wherein
A5 is N;
one of A6 and A7 is CH and the other one is N; or one of A6 and A7 is C and takes part in the annulated carbo- or heterocycle Z and the other one is N;
A8 is Nor CH.

8. The inhibitor of claim 6, with a structure according to formula (II), wherein
A5 is N;
one of A6 and A7 is C and takes part in the annulated 5-, 6- or 7-membered carbo- or heterocycle Z and the other one is N;
A8 is N;
L2 is CH₂—CH₂—R4 and L3 is CH₂—CH₂—R5 or CH₂—CF₂—R5 or L2 and L3 together with the A8 to which they are connected form a 5- or 6-membered heterocycle substituted by R4 and R5;
L4 is absent;
Z is any 6-membered carbo- or heterocycle annulated to the central core, wherein Z is optionally substituted with one, two, or three substituent(s) selected from the group consisting of —Br, —Cl, —F, —I, —OH, Me, —CF₃, Et, —OMe, —SMe, and NO₂;
R4 is COOH or tetrazolyl;
R5 is any 5-, 6-, or 7-membered carbo- or heterocycle, optionally substituted with one, two, or three lipophilic substituent(s) selected from the group consisting of —Br, —Cl, —F, —I, Me, —CF₃, Et, —OMe, and —SMe;
R6 is any 5- or 6-membered carbo- or heterocycle, optionally substituted with one, two, or three substituents selected from the group consisting of —Br, —Cl, —F, —I, —OH, —NO₂, Me, —CF₃, Et, —OMe, and —SMe,
or R6 is H.

9. The inhibitor of claim 6, with a structure according to formula (II), wherein
each one of A5, A7 and A8 is N;
A6 is C and takes part in the annulated carbo- or heterocycle Z;
each one of L2 and L3 is CH₂—CH₂; or L2 and L3 together with the A8 to which they are connected form a piperidine ring or a pyrrolidine ring, substituted by R4 and R5;
L4 is absent;
Z is phenyl or cyclohexyl annulated to the central core, wherein Z is optionally substituted with one, two, or three substituent(s) selected from the group consisting of —Br, —Cl, —F, —OH, Me, —CF₃, —OMe, and —NO₂,
R4 is COOH or tetrazolyl;
R5 is phenyl, cyclopentyl or adamantyl, optionally substituted with one, two, or three lipophilic substituent(s) selected from the group consisting of —Br, —CF₃, Me, —CH₂—CF₃, and —OMe;
R6 is any 6-membered carbo- or heterocycle, optionally substituted with one, two, or three substituent(s) selected from the group consisting of Br, —Cl, —F, Me, —CF₃, —OMe, and —NO₂.

10. The inhibitor of claim 1, having a structure according to formula (IV): 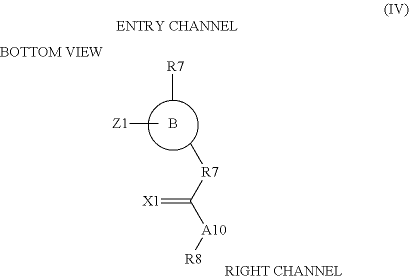
wherein
B is a 5-, 6-, or 7-membered heterocycle comprising 1, 2, or 3 heteroatoms selected from O, N, or S;
R7 is any 5-, 6- or 7-membered carbo- or heterocycle, optionally substituted with one, two, or three lipophilic substituent(s) selected from the group consisting of —Br, —Cl, —F, —I, Me, Et, —OMe, and —SMe;
Z1 is selected from the group consisting of —Br, —Cl, —F, —I, —OH, —NH₂, Me, —CF₃, Et, —OMe, —SMe, and —NO₂; or Z1 is absent,
X1 is O or S;
A9 is O, NH or CH₂;
A10 is O, NH or CH₂; and
R8 is any 5-, 6- or 7-membered carbo- or heterocycle, optionally substituted with one, two, or three lipophilic substituent(s) selected from the group consisting of —Br, —Cl, —F, —I, Me, Et, —OMe, and —SMe;
or a pharmaceutically acceptable salt, isomer, solvate, chemically protected form, or prodrug thereof.

11. The inhibitor of claim 10, wherein
X1 is O;
A9 is O or NH; and
A10 is O or NH
or
X1 is O or S;
A9 is NH; and
A10 is NH.

12. The inhibitor of claim 10, having a structure according to formula (III) 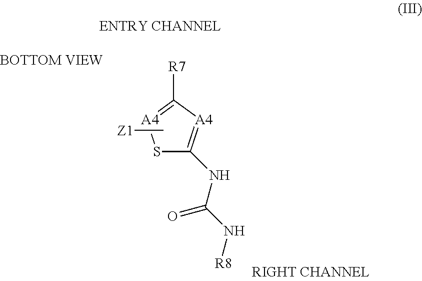
wherein
each A4 is independently from each other selected from N, NH, CH or CH₂;
Z1 is selected from the group consisting of —Br, —Cl, —F, —I, —OH, —NH₂, Me, —CF₃, Et, —OMe, —SMe, and —NO₂; or Z1 is absent;
R7 is any 5-, 6- or 7-membered carbo- or heterocycle, optionally substituted with one, two, or three lipophilic substituent(s) selected from the group consisting of —Br, —Cl, —F, —I, Me, Et, —OMe, and —SMe;
R8 is any 5-, 6- or 7-membered carbo- or heterocycle, optionally substituted with one, two, or three lipophilic substituent(s) selected from the group consisting of —Br, —Cl, —F, —I, Me, Et, —OMe, and —SMe;
or a pharmaceutically acceptable salt, isomer, solvate, chemically protected form, or prodrug thereof.

13. The inhibitor of claim 12, with a structure according to formula (III), wherein
each A4 is N,
Z1 is selected from the group consisting of —Br, —Cl, —F, —I, —OH, —NH₂, Me, —CF₃, Et, —OMe, —SMe, and —NO₂; or Z1 is absent;
R8 is cyclohexyl, piperidine, hexahydropyridazine, hexahydropyrimidine, or piperazinyl, optionally substituted with one, two, or three lipophilic substituent(s) selected from the group consisting of —Br, —Cl, —F, —I, Me, Et, —OMe, and —SMe;
R7 is a 5- or 6-membered carbo- or heterocycle, optionally substituted with a methyl group or an ethyl group.

14. The inhibitor of claim 1, selected from the group consisting of: 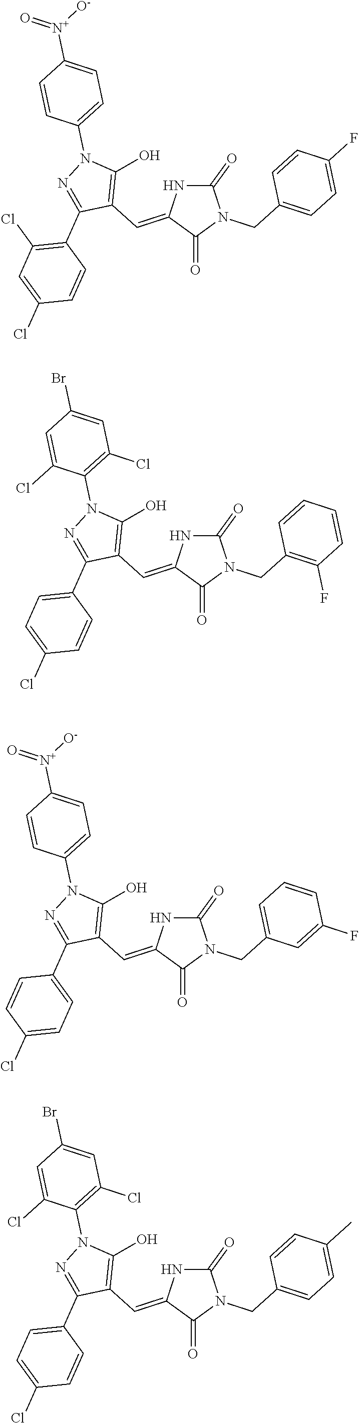 [SMILES: O=C1N/C(=C\c2c(-c3ccc(Cl)cc3Cl)nn(-c3ccc([N+](=O)[O-])cc3)c2O)C(=O)N1Cc1ccc(F)cc1 ; O=C1N/C(=C\c2c(-c3ccc(Cl)cc3)nn(-c3c(Cl)cc(Br)cc3Cl)c2O)C(=O)N1Cc1ccccc1F ; O=C1N/C(=C\c2c(-c3ccc(Cl)cc3)nn(-c3ccc([N+](=O)[O-])cc3)c2O)C(=O)N1Cc1cccc(F)c1 ; Cc1ccc(CN2C(=O)N/C(=C\c3c(-c4ccc(Cl)cc4)nn(-c4c(Cl)cc(Br)cc4Cl)c3O)C2=O)cc1] 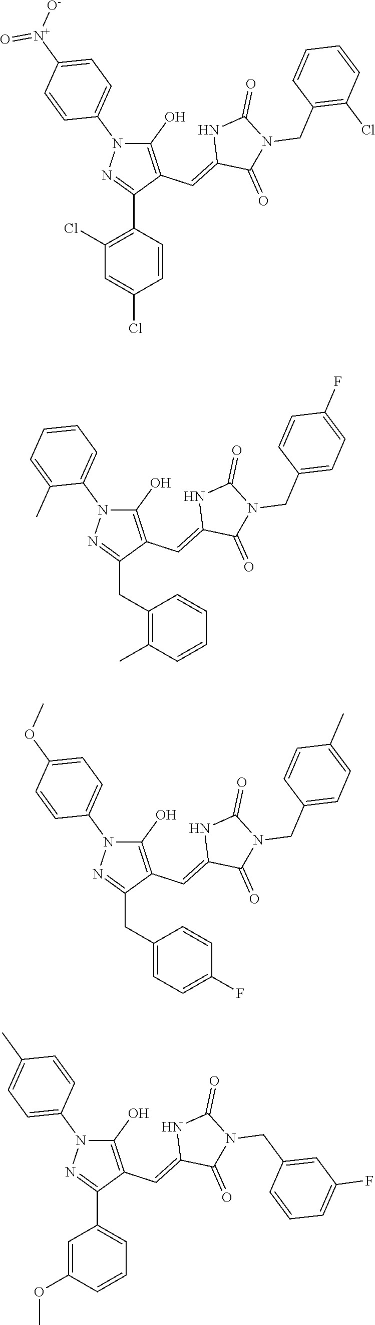 [SMILES: O=C1N/C(=C\c2c(-c3ccc(Cl)cc3Cl)nn(-c3ccc([N+](=O)[O-])cc3)c2O)C(=O)N1Cc1ccccc1Cl ; Cc1ccccc1Cc1nn(-c2ccccc2C)c(O)c1/C=C1\NC(=O)N(Cc2ccc(F)cc2)C1=O ; COc1ccc(-n2nc(Cc3ccc(F)cc3)c(/C=C3\NC(=O)N(Cc4ccc(C)cc4)C3=O)c2O)cc1 ; COc1cccc(-c2nn(-c3ccc(C)cc3)c(O)c2/C=C2\NC(=O)N(Cc3cccc(F)c3)C2=O)c1] 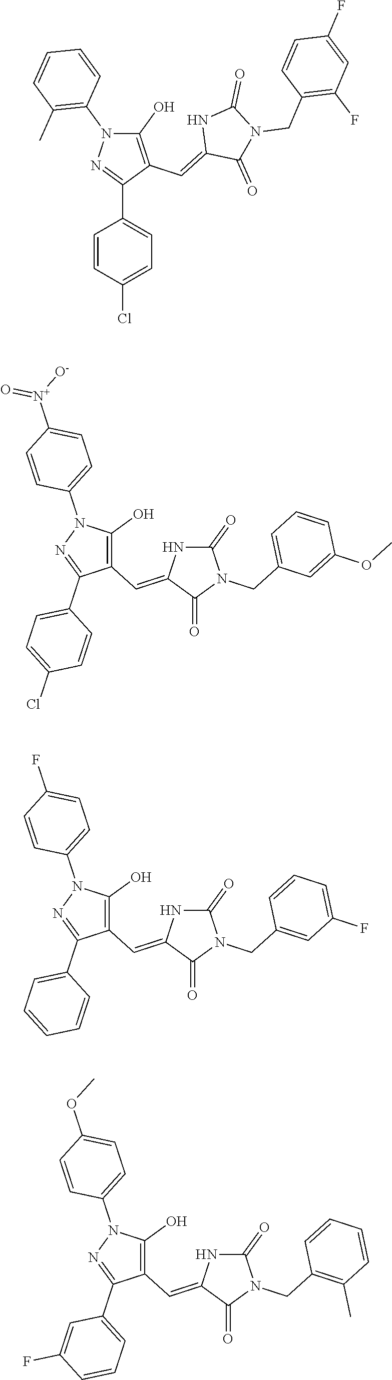 [SMILES: Cc1ccccc1-n1nc(-c2ccc(Cl)cc2)c(/C=C2\NC(=O)N(Cc3ccc(F)cc3F)C2=O)c1O ; COc1cccc(CN2C(=O)N/C(=C\c3c(-c4ccc(Cl)cc4)nn(-c4ccc([N+](=O)[O-])cc4)c3O)C2=O)c1 ; O=C1N/C(=C\c2c(-c3ccccc3)nn(-c3ccc(F)cc3)c2O)C(=O)N1Cc1cccc(F)c1 ; COc1ccc(-n2nc(-c3cccc(F)c3)c(/C=C3\NC(=O)N(Cc4ccccc4C)C3=O)c2O)cc1] 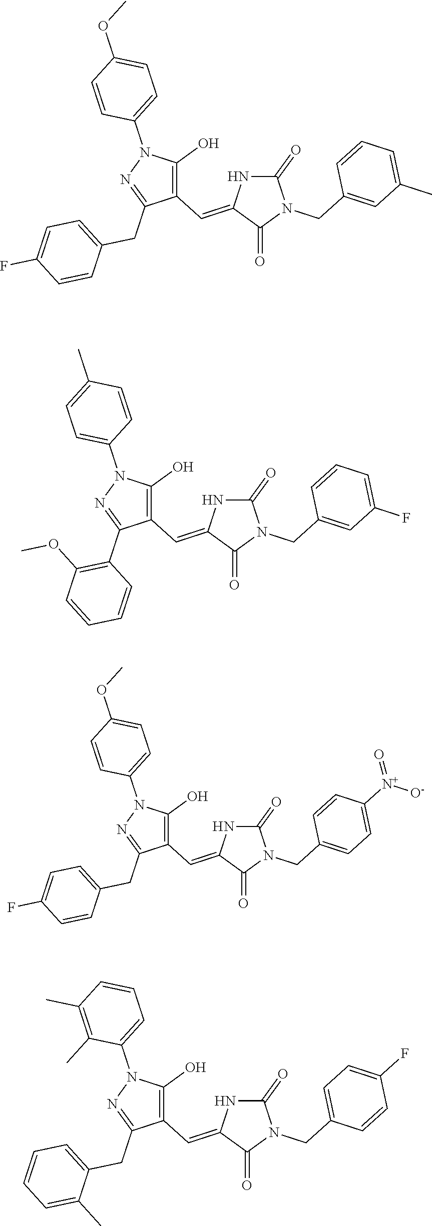 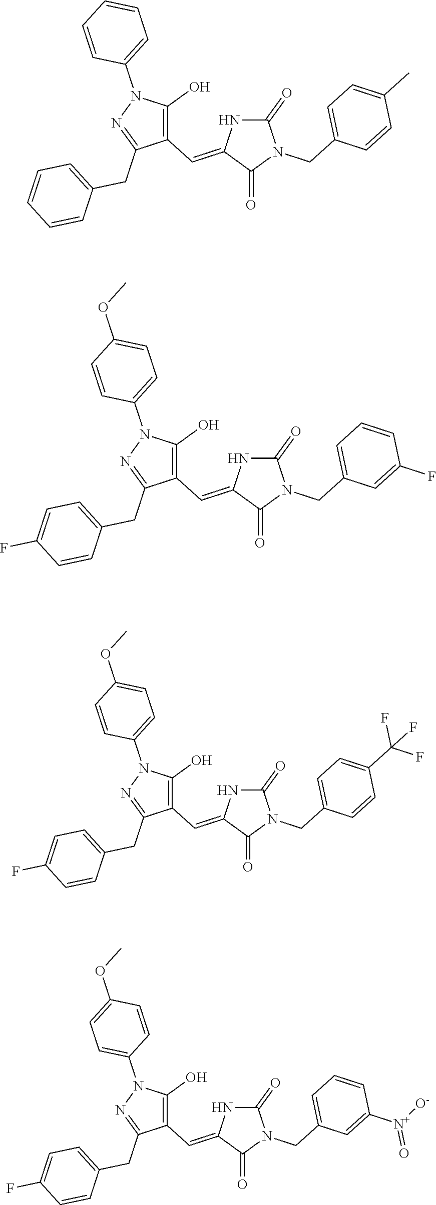 [SMILES: Cc1ccc(CN2C(=O)N/C(=C\c3c(Cc4ccccc4)nn(-c4ccccc4)c3O)C2=O)cc1 ; COc1ccc(-n2nc(Cc3ccc(F)cc3)c(/C=C3\NC(=O)N(Cc4cccc(F)c4)C3=O)c2O)cc1 ; COc1ccc(-n2nc(Cc3ccc(F)cc3)c(/C=C3\NC(=O)N(Cc4ccc(C(F)(F)F)cc4)C3=O)c2O)cc1 ; COc1ccc(-n2nc(Cc3ccc(F)cc3)c(/C=C3\NC(=O)N(Cc4cccc([N+](=O)[O-])c4)C3=O)c2O)cc1] 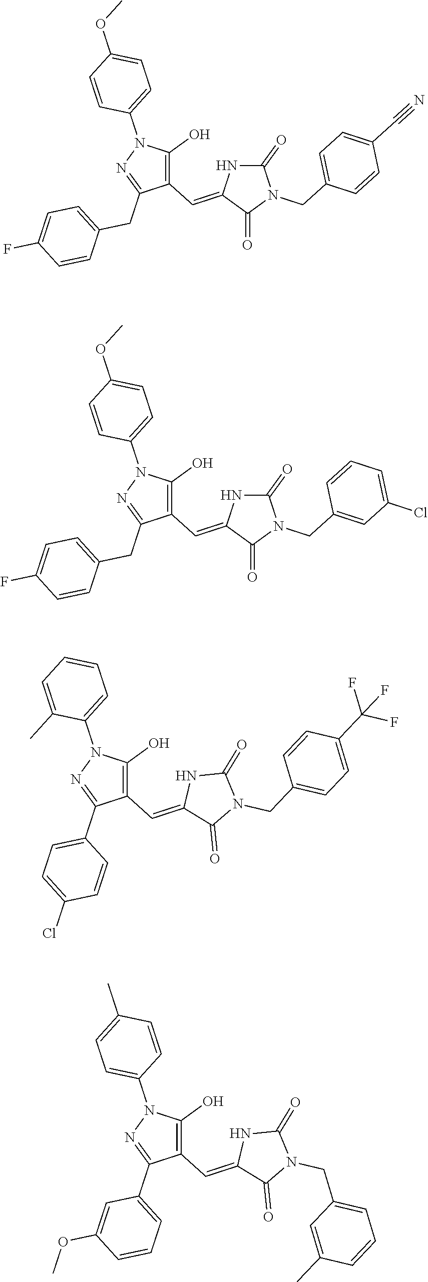 [SMILES: COc1ccc(-n2nc(Cc3ccc(F)cc3)c(/C=C3\NC(=O)N(Cc4ccc(C#N)cc4)C3=O)c2O)cc1 ; COc1ccc(-n2nc(Cc3ccc(F)cc3)c(/C=C3\NC(=O)N(Cc4cccc(Cl)c4)C3=O)c2O)cc1 ; Cc1ccccc1-n1nc(-c2ccc(Cl)cc2)c(/C=C2\NC(=O)N(Cc3ccc(C(F)(F)F)cc3)C2=O)c1O ; COc1cccc(-c2nn(-c3ccc(C)cc3)c(O)c2/C=C2\NC(=O)N(Cc3cccc(C)c3)C2=O)c1] 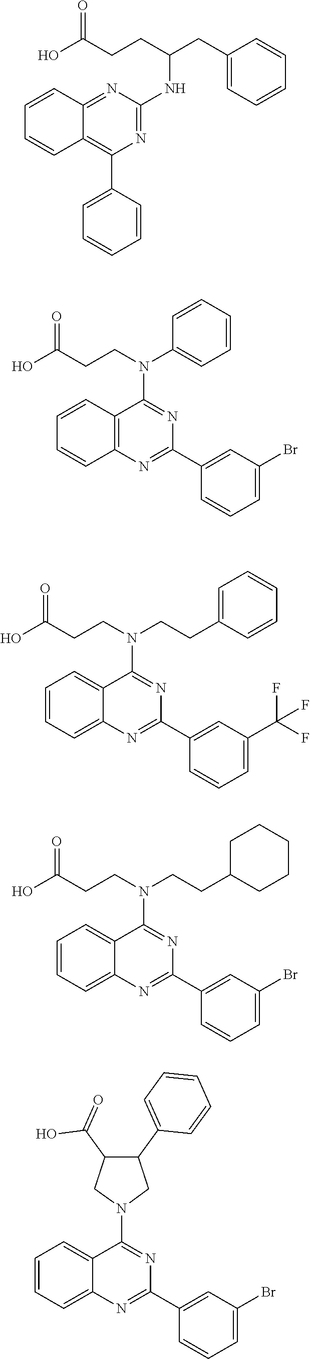 [SMILES: O=C(O)CCC(Cc1ccccc1)Nc1nc(-c2ccccc2)c2ccccc2n1 ; O=C(O)CCN(c1ccccc1)c1nc(-c2cccc(Br)c2)nc2ccccc12 ; O=C(O)CCN(CCc1ccccc1)c1nc(-c2cccc(C(F)(F)F)c2)nc2ccccc12 ; O=C(O)CCN(CCC1CCCCC1)c1nc(-c2cccc(Br)c2)nc2ccccc12 ; O=C(O)C1CN(c2nc(-c3cccc(Br)c3)nc3ccccc23)CC1c1ccccc1] 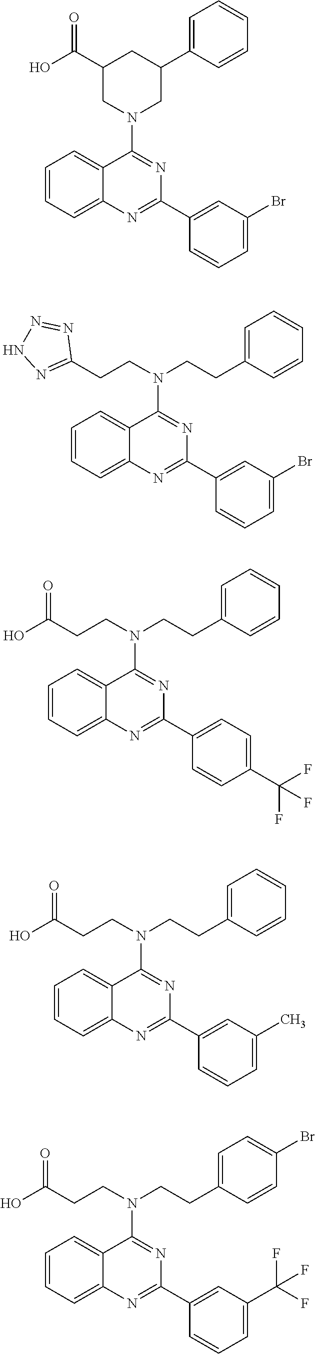 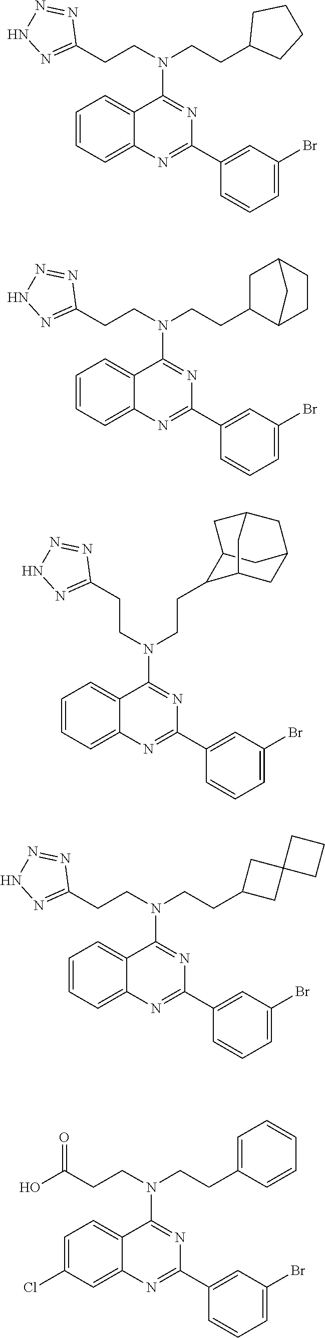 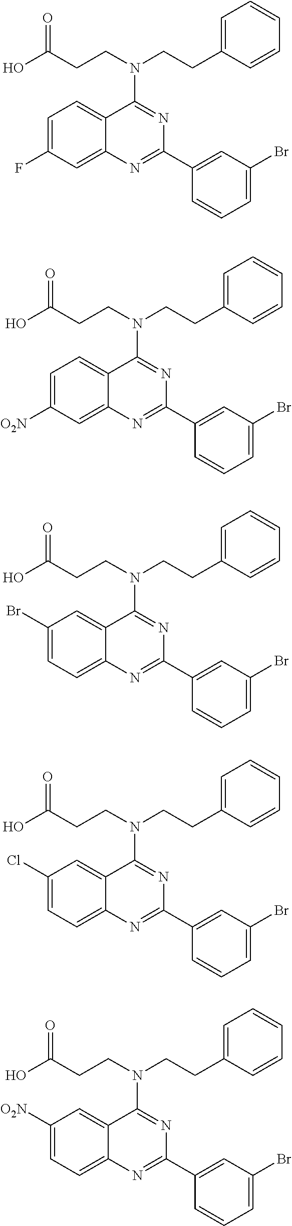 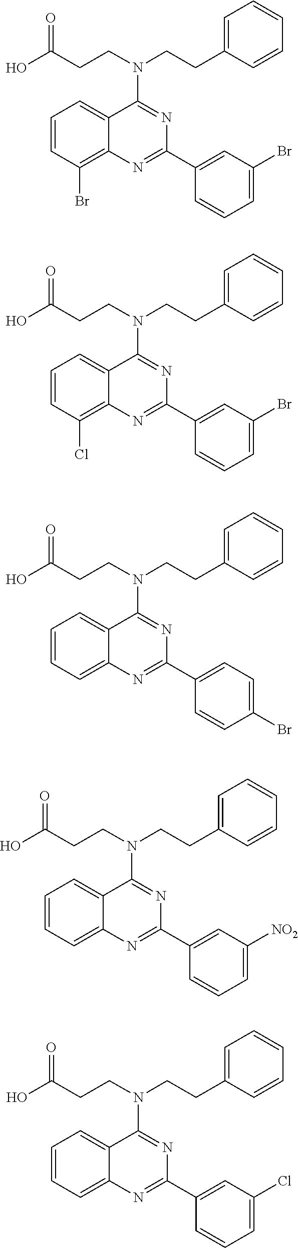 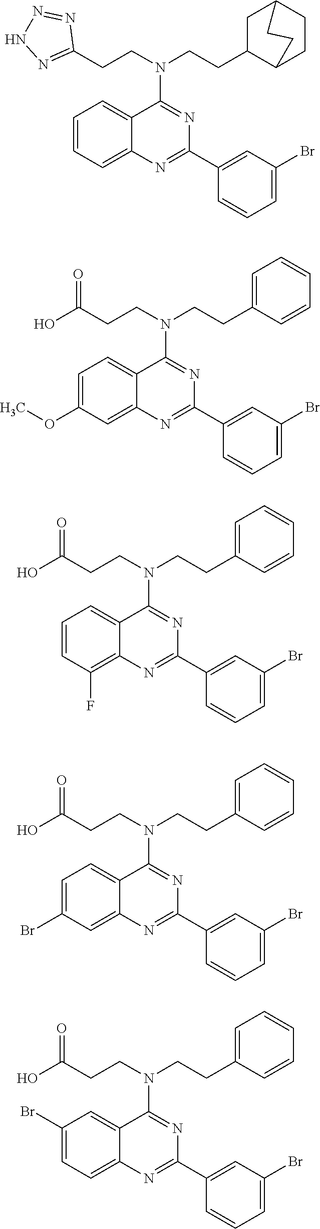 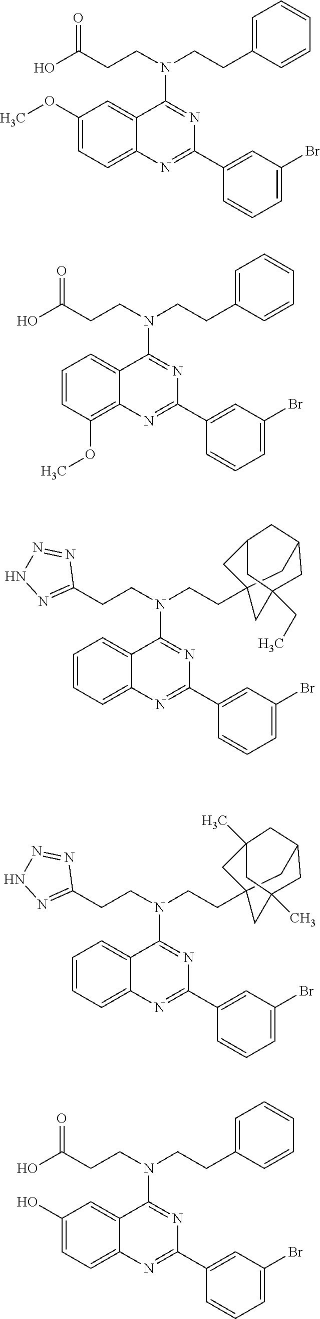 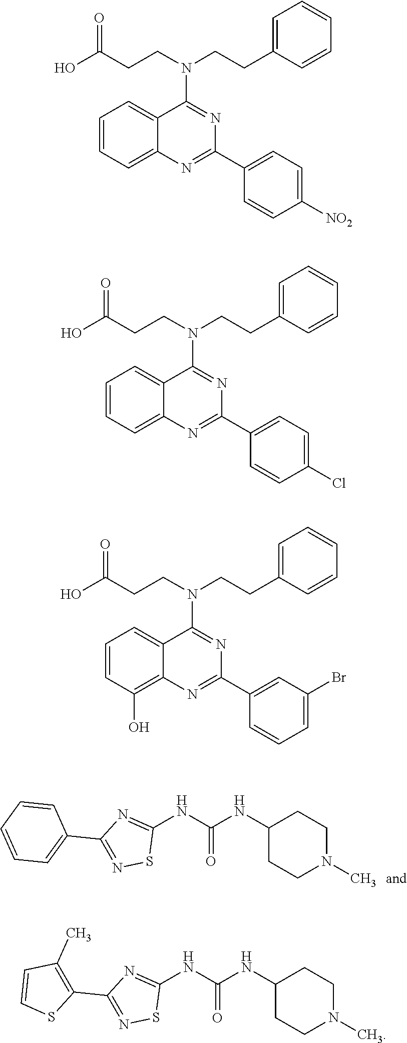 [SMILES: O=C(O)CCN(CCc1ccccc1)c1nc(-c2ccc([N+](=O)[O-])cc2)nc2ccccc12 ; O=C(O)CCN(CCc1ccccc1)c1nc(-c2ccc(Cl)cc2)nc2ccccc12 ; O=C(O)CCN(CCc1ccccc1)c1nc(-c2cccc(Br)c2)nc2c(O)cccc12 ; CN1CCC(NC(=O)Nc2nc(-c3ccccc3)ns2)CC1 ; "and" ; Cc1ccsc1-c1nsc(NC(=O)NC2CCN(C)CC2)n1]
or pharmaceutically acceptable salt, isomer, solvate, chemically protected form, or prodrug thereof.

15. The inhibitor of claim 1, selected from the group consisting of

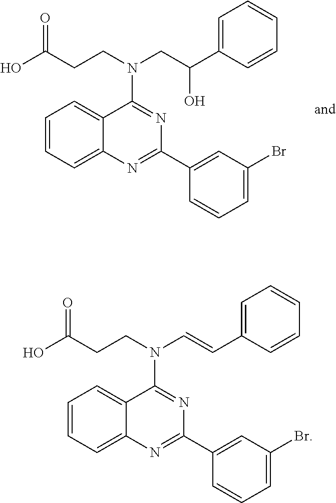 [SMILES: O=C(O)CCN(CC(O)c1ccccc1)c1nc(-c2cccc(Br)c2)nc2ccccc12 ; O=C(O)CCN(/C=C/c1ccccc1)c1nc(-c2cccc(Br)c2)nc2ccccc12]

16. A pharmaceutical composition comprising the inhibitor according to claim 1.

17. A method for treating or preventing a viral infection in a patient comprising administering to the patient an inhibitor according to claim 1.

18. The method according to claim 17, wherein the viral infection is an infection with a Pisoniviricetes.
